SEQUENCE LISTING (24 sequences)

SEQ ID NO: 1 - protein, 31 aa, synthetic construct
  source 1..31: note = Heterologous sequence
KDEPQRRSARLSAKPAPPKPEPKPKKAPAKK

SEQ ID NO: 2 - protein, 11 aa, synthetic construct
  source 1..11: note = Heterologous sequence
KDEPQRRSARL

SEQ ID NO: 3 - protein, 10 aa, synthetic construct
  source 1..10: note = Heterologous sequence
SAKPAPPKPE

SEQ ID NO: 4 - protein, 10 aa, synthetic construct
  source 1..10: note = Heterologous sequence
PKPKKAPAKK

SEQ ID NO: 5 - protein, 736 aa, synthetic construct
  source 1..736: note = AAV9 wild-type (WT) capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - DNA, 2211 bp, synthetic construct
  source 1..2211: mol_type = other DNA; note = AAV9 capsid
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 7 - protein, 738 aa, synthetic construct
  source 1..738: note = AAV8 WT capsid protein
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 8 - DNA, 30 bp, synthetic construct
  source 1..30: mol_type = other DNA; note = SAKPAPPKPE nucleotide sequence
tccgcaaagccagcgccaccaaagcccgag

SEQ ID NO: 9 - DNA, 2241 bp, synthetic construct
  source 1..2241: mol_type = other DNA; note = Modified AAV9 capsid (VP1) nucleotide sequence (SAKPAPPKPE peptide inserted at aa position 589/VR-VIII)
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatccgcaaagccagcgccaccaaagcccgaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 10 - protein, 746 aa, synthetic construct
  source 1..746: note = Modified AAV9 capsid (VP1) protein with SAKPAPPKPE peptide insert at aa 589
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSAKPAPPKPEAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - DNA, 2241 bp, synthetic construct
  source 1..2241: mol_type = other DNA; note = Modified AAV9 capsid (VP1) nucleotide sequence (SAKPAPPKPE peptide inserted at aa position 455/VR-IV)
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttcttccgcaaagccagcgccaccaaagcccgagggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 12 - protein, 746 aa, synthetic construct
  source 1..746: note = Modified AAV9 (VP1) protein with SAKPAPPKPE peptide insert at aa 455
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSSAKPAPPKPEGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 746 aa, synthetic construct
  source 1..746: note = Modified AAV9 (VP1) protein with SAKPAPPKPE peptide insert at aa 533
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDSAKPAPPKPERFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 747 aa, synthetic construct
  source 1..747: note = Modified AAV9(VP1) protein with KDEPQRRSARL peptide insert at aa 455
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSKDEPQRRSARLGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 747 aa, synthetic construct
  source 1..747: note = Modified AAV9 (VP1) protein with KDEPQRRSARL peptide insert at aa 533
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDKDEPQRRSARLRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 747 aa, synthetic construct
  source 1..747: note = Modified AAV9 capsid (VP1) protein with KDEPQRRSARL peptide insert at aa 589
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQKDEPQRRSARLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 746 aa, synthetic construct
  source 1..746: note = Modified AAV9(VP1) protein with PKPKKAPAKK peptide insert at aa 455
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSPKPKKAPAKKGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - protein, 746 aa, synthetic construct
  source 1..746: note = Modified AAV9 (VP1) protein with PKPKKAPAKK peptide insert at aa 533
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDPKPKKAPAKKRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 746 aa, synthetic construct
  source 1..746: note = Modified AAV9 capsid (VP1) protein with PKPKKAPAKK peptide insert at aa 589
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPKPKKAPAKKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 20 - protein, 767 aa, synthetic construct
  source 1..767: note = Modified AAV9(VP1) protein with KDEPQRRSARLSAKPAPPKPEPKPKKAPAKK peptide insert at aa 455
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSKDEPQRRSARLSAKPAPPKPEPKPKKAPAKKGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 21 - protein, 767 aa, synthetic construct
  source 1..767: note = Modified AAV9 (VP1) protein with KDEPQRRSARLSAKPAPPKPEPKPKKAPAKK peptide insert at aa 533
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDKDEPQRRSARLSAKPAPPKPEPKPKKAPAKKRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 767 aa, synthetic construct
  source 1..767: note = Modified AAV9 capsid (VP1) protein with KDEPQRRSARLSAKPAPPKPEPKPKKAPAKK peptide insert at aa 589
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQKDEPQRRSARLSAKPAPPKPEPKPKKAPAKKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 32 aa, synthetic construct
  source 1..32: note = Heterologous sequence
MKDEPQRRSARLSAKPAPPKPEPKPKKAPAKK

SEQ ID NO: 24 - protein, 743 aa, synthetic construct
  source 1..743: note = AAV2 variant (Reference capsid)
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTR

(19) United States
(12) Patent Application Publication
(10) Pub. No.: US 2025/0249127 A1
(43) Pub. Date: Aug. 7, 2025

(54) GENE THERAPY FOR TREATING AN OCULAR DISEASE

(71) Applicant: REGENXBIO INC., Rockville, MD (US)

(72) Inventors: Samantha YOST, Clarksburg, MD (US); Ye LIU, Clarksville, MD (US); Olivier DANOS, Princeton, NJ (US); Andrew MERCER, Poolesville, MD (US); Wei-Hua LEE, Darnestown, MD (US); Xu WANG, Falls Church, VA (US)

(21) Appl. No.: 18/856,534

(22) PCT Filed: Apr. 13, 2023
(86) PCT No.: PCT/US2023/065740

§ 371 Date: Oct. 11, 2024

Related U.S. Application Data
(60) Provisional application No. 63/330,984, filed on Apr. 14, 2022.
(60) Provisional application No. 63/342,403, filed on May 16, 2022.

(51) Int. Cl. A61K 48/00; C07K 14/005; C12N 15/86

(52) U.S. Cl. CPC A61K 48/0058; C07K 14/005; C12N 15/86; C12N 2750/14122; C12N 2750/14143

(57) ABSTRACT

Provided herein are gene therapy methods for the treatment of diseases or disorders associated with the eye of a subject involving the use of recombinant adeno-associated viruses (rAAVs) comprising a variant AAV capsid to deliver a transgene to the eye of the subject. Also provided herein are rAAVs and compositions comprising the rAAVs that can be used in the gene therapy methods of the disclosure, and methods of making such rAAVs.

### CROSS-REFERENCE TO RELATED APPLICATIONS

This application claims the benefit of U.S. Provisional Patent Application No. 63/330,984, filed Apr. 14, 2022, and U.S. Provisional Patent Application No. 63/342,403, filed May 16, 2022, the content of each of which is incorporated herein by reference in its entirety.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application entitled “12656-166-228_SEQ_LISTING.xml”, was created on Apr. 11, 2023 and is 44,749 bytes in size.

### 1. FIELD

The field relates to the treatment of a disease or disorder associated with the eye of a subject (e.g., retinal disease). Provided herein are methods and compositions involving recombinant adeno-associated viruses (rAAVs) for the treatment of a disease or disorder associated with the eye of a subject. Provided herein are recombinant adeno-associated viruses (rAAVs) having capsid proteins engineered to include amino acid sequences that confer and/or enhance desired properties. For example, provided herein are engineered capsid proteins comprising peptide insertions within or near variable region VR-VIII, VR-IV, and/or VR-VI of the virus capsid (e.g., AAV9). Also provided herein are capsid proteins that direct rAAVs to target tissues (e.g., ocular tissue) or location (e.g., targeted to the eye).

### 2. BACKGROUND

The use of adeno-associated viruses (AAV) as gene delivery vectors is a promising avenue for the treatment of many unmet patient needs. Dozens of naturally occurring AAV capsids have been reported, and mining the natural diversity of AAV sequences in primate tissues has identified over a hundred variants, distributed in clades. AAVs belong to the parvovirus family and are single-stranded DNA viruses with relatively small genomes and simple genetic components. Without a helper virus, AAV establishes a latent infection. An AAV genome generally has a Rep gene and a Cap gene, flanked by inverted terminal repeats (ITRs), which serve as replication and packaging signals for vector production. The capsid proteins form capsids that carry genome DNA and can determine tissue tropism to deliver DNA into target cells.

Due to low pathogenicity and the promise of long-term, targeted gene expression, recombinant AAVs (rAAVs) have been used as gene transfer vectors, in which therapeutic sequences are packaged into various capsids. Such vectors have been used in preclinical gene therapy studies and several gene therapy products are currently in clinical development. However, there remains a need for rAAV vectors that achieve high transduction efficiency in the eye (e.g., posterior segment of the eye, such as retina and retinal pigment epithelium (RPE)). There also is a need for rAAV vectors with enhanced tissue-specific targeting, cell-specific tropism, and/or enhanced tissue-specific transduction to deliver therapies (e.g., directed to the eye).

### 3. SUMMARY

In one aspect provided herein is a recombinant adeno-associated virus (rAAV) vector comprising a variant AAV capsid protein, wherein the variant AAV capsid protein comprises a heterologous amino acid sequence having at least about 90% sequence identity to at least about 10 contiguous amino acids of SEQ ID NO: 23, wherein the heterologous amino acid sequence is inserted at the VR-IV site, the VR-VIII site, or the VR-VI site of an AAV capsid protein. In various aspects, SEQ ID NO: 23 is MKDEPQRRSARLSAKPAPPKPEPKPKKAPAKK.

In one aspect provided herein is a pharmaceutical composition suitable for administration to the eye of a subject, wherein the pharmaceutical composition comprises a recombinant adeno-associated virus (rAAV) vector comprising a variant AAV capsid protein, wherein the variant AAV capsid protein comprises a heterologous amino acid sequence relative to a corresponding parental AAV capsid protein, wherein the heterologous amino acid sequence comprises at least about 10 contiguous amino acids of SEQ ID NO: 23.

In one aspect provided herein is a method of treating a disease associated with the eye of a subject, wherein the method comprises administering a recombinant adeno-associated virus (rAAV) vector to the eye of the subject, wherein the rAAV comprises a variant AAV capsid protein, wherein the variant AAV capsid protein comprises a heterologous amino acid sequence relative to a corresponding parental AAV capsid protein, wherein the heterologous amino acid sequence comprises at least about 10 contiguous amino acids of SEQ ID NO: 23.

In one aspect provided herein is an isolated nucleic acid comprising a nucleotide sequence encoding a variant adeno-associated virus (AAV) capsid protein, wherein the variant AAV capsid protein comprises a heterologous amino acid sequence having at least about 90% sequence identity to at least about 10 contiguous amino acids of SEQ ID NO: 23, wherein the heterologous amino acid sequence is inserted at the VR-IV site, the VR-VIII site, or the VR-VI site of an AAV capsid protein.

In one aspect provided herein is a method of treating a disease associated with the CNS of a subject, wherein the method comprises administering a recombinant adeno-associated virus (rAAV) vector to the CNS of the subject, wherein the rAAV vector comprises a variant AAV capsid protein encapsidating a transgene, wherein the variant AAV capsid protein comprises a heterologous amino acid sequence relative to a corresponding parental AAV capsid protein, wherein the heterologous amino acid sequence comprises at least about 10 contiguous amino acids of SEQ ID NO: 23.

In some aspects, the at least about 10 contiguous amino acids of SEQ ID NO: 23 comprises SEQ ID NO: 2. In some aspects, the at least about 10 contiguous amino acids of SEQ ID NO: 23 consists of SEQ ID NO: 2. In various aspects, SEQ ID NO: 2 is KDEPQRRSARL. In some aspects, the at least about 10 contiguous amino acids of SEQ ID NO: 23 comprises SEQ ID NO: 3. In some aspects, the at least about 10 contiguous amino acids of SEQ ID NO: 23 consists of SEQ ID NO: 3. In various aspects, SEQ ID NO: 3 is SAKPAPPKPE. In some aspects, the at least about 10 contiguous amino acids of SEQ ID NO: 23 comprises SEQ ID NO: 4. In some aspects, the at least about 10 contiguous amino acids of SEQ ID NO: 23 consists of SEQ ID NO: 4. In various aspects provided herein, SEQ ID NO: 4 is PKPKKAPAKK. In some aspects, the at least about 10 contiguous amino acids of SEQ ID NO: 23 comprises SEQ ID NO: 1. In some aspects, the at least about 10 contiguous amino acids of SEQ ID NO: 23 consists of SEQ ID NO: 1. In some aspects, the at least about 10 contiguous amino acids of SEQ ID NO: 23 comprises SEQ ID NO: 23. In some aspects, the at least about 10 contiguous amino acids of SEQ ID NO: 23 consists of SEQ ID NO: 23.

In some aspects, the heterologous amino acid sequence comprises any one of SEQ ID NOs: 1-4 and 23. In some aspects, the heterologous amino acid sequence consists of any one of SEQ ID NOs: 1-4 and 23.

In some aspects, the at least about 10 contiguous amino acids of SEQ ID NO: 23 is inserted in at least one position selected from positions 455, 533, and/or 589 of AAV9 or the corresponding position in the capsid protein of another AAV serotype. In some aspects, the heterologous amino acid sequence is inserted at the VR-IV site. In some aspects, the heterologous amino acid sequence is inserted at the VR-IV site at amino acid position 455 of a parental AAV9 or a corresponding position in another AAV serotype. In some aspects, the heterologous amino acid sequence is inserted at the VR-VI site. In some aspects, the heterologous amino acid sequence is inserted at the VR-VI site at amino acid position 533 of a parental AAV9 or a corresponding position in another AAV serotype. In some aspects, the heterologous amino acid sequence is inserted at the VR-VIII site. In some aspects, the heterologous amino acid sequence is inserted at the VR-VIII site at amino acid position 588 of a parental AAV9 or a corresponding position in another AAV serotype.

In some aspects, the rAAV is suitable for administration to the eye of a subject.

In some aspects, the rAAV further comprises a transgene.

In some aspects, the method results in a homogeneous expression of the transgene in the retina of the subject after the rAAV vector is administered to the eye of the subject. In some aspects, the homogeneous expression is measured using a fluorescence assay. In some aspects, the fluorescence assay is fluorescence resonance energy transfer (FRET).

In some aspects, the rAAV is administered intravitreally to the eye of the subject.

In some aspects, the variant AAV capsid protein confers increased infectivity of a retinal cell compared to the infectivity of the retinal cell by an AAV vector comprising the corresponding parental AAV capsid protein, wherein the AAV vector does not comprise the heterologous amino acid sequence.

In some aspects, the subject is a human subject. In some aspects, the subject is a subject diagnosed with or suspected of having an ocular disease. In some aspects, the ocular disease is a retinal disease. In some aspects, the disease is a retinal disease. In some aspects, the retinal disease is selected from age-related macular degeneration, retinal vasculitis and retinal infective processes, commotio retinae, diabetic retinopathy, hereditary retinal dystrophies, ischemic insult of retinal neurons and macular edema.

In some aspects, the variant AAV capsid protein is identical to an AAV9 capsid protein except for the at least about 10 contiguous amino acids of SEQ ID NO: 23.

In some aspects, the isolated nucleic acid further encodes a transgene.

In some aspects, the variant capsid protein when present in an AAV vector, provides for increased infectivity of the AAV vector for a retinal cell. In some aspects, the administering is via subretinal, intravitreal or suprachoroidal administration to the eye of the subject. In some aspects, the pharmaceutical composition is suitable for subretinal, intravitreal or suprachoroidal administration to the eye of the subject.

In some aspects, the method results in higher transgene expression level in a portion of the eye of the subject after the rAAV is administered to the eye of the subject compared to the transgene expression level after an AAV vector comprising the corresponding parental AAV capsid protein and the transgene is administered to the eye of the subject or a comparable subject, wherein the AAV vector comprising the corresponding parental AAV capsid protein and the transgene does not comprise the heterologous amino acid sequence. In some aspects, the transgene expression level is higher by about or at least about 5%, 10%, 15%, 20%, 25%, 30%, 35%, 40%, 45%, 50%, 55%, 60%, 65%, 70%, 75%, 80%, 85%, 90%, 95%, 99%, or 100%.

In some aspects, the portion of the eye is the retina. In some aspects, the portion of the eye is retinal pigment epithelium (RPE), choroid, and/or sclera. In some aspects, the portion of the eye is the anterior segment. In some aspects, the portion of the eye is a population of photoreceptors. In some aspects, the rAAV comprises SEQ ID NO: 3 after position Q588 of AAV9. In some aspects, the rAAV comprises SEQ ID NO: 3 after position S454 of AAV9. In some aspects, the corresponding parental AAV capsid protein is AAV9 capsid protein.

In some aspects, the rAAV and/or the AAV vector is administered by intravitreal administration.

In some aspects, the ocular structural integrity of the eye remains intact at least about 1 week after the rAAV vector is administered to the eye of the subject as determined by optical coherence tomography (OCT).

In some aspects, the comparable subject has been diagnosed with or is at risk of developing the same disease associated with the eye as the subject.

In some aspects, the transgene expression level is determined by an enzyme linked immunosorbent assay (ELISA).

In some aspects, the rAAV vector is administered intravenously, intrathecally, intracerebroventicularly, or intraparenchymally.

In some aspects, the parental AAV capsid protein is rAAV9.

In some aspects, the method results in a focal expression of the transgene in the CNS tissue of the injection site after the rAAV vector is administered via intraparenchymal injection.

In one aspect provided herein is a recombinant adeno-associated virus (rAAV) vector comprising: a nucleic acid sequence encoding an adeno-associated virus (AAV) capsid protein comprising an amino acid sequence that is at least 95% identical to any one of SEQ ID NOs: 10 and 12-22.

In some aspects, the AAV capsid protein comprises an amino acid sequence that is at least 98% identical to any one of SEQ ID NOs: 10 and 12-22. In some aspects, the AAV capsid protein comprises an amino acid sequence that is identical to any one of SEQ ID NOs: 10 and 12-22. In some aspects, the AAV capsid protein comprises an amino acid sequence that is identical to SEQ ID NO: 10. In some aspects, the AAV capsid protein comprises an amino acid sequence that is identical to SEQ ID NO: 12. In some aspects, the nucleic acid sequence encoding the AAV capsid protein comprises a nucleotide sequence that is identical to SEQ ID NO: 9 or 11. In some aspects, the nucleic acid sequence encoding the AAV capsid protein comprises a nucleotide sequence of SEQ ID NOs: 9. In some aspects, the nucleic acid sequence encoding the AAV capsid protein comprises a nucleotide sequence of SEQ ID NOs: 11.

In one aspect provided herein is a pharmaceutical composition suitable for administration to the eye of a subject, wherein the pharmaceutical composition comprises a recombinant adeno-associated virus (rAAV) vector comprising a variant AAV capsid protein, wherein the variant AAV capsid protein comprises an amino acid sequence that is at least 95% identical to any one of SEQ ID NOs: 10 and 12-22.

In some aspects, the variant AAV capsid protein comprises an amino acid sequence that is identical to any one of SEQ ID NOs: 10 and 12-22. In some aspects, the variant AAV capsid protein comprises an amino acid sequence that is identical to SEQ ID NO: 10. In some aspects, the variant AAV capsid protein comprises an amino acid sequence that is identical to SEQ ID NO: 12.

In one aspect provided herein is a method of treating a disease associated with the eye of a subject, wherein the method comprises administering a recombinant adeno-associated virus (rAAV) vector to the eye of the subject, wherein the rAAV comprises a variant AAV capsid protein, wherein the variant AAV capsid protein comprises an amino acid sequence that is at least 95% identical to any one of SEQ ID NOs: 10 and 12-22.

In one aspect provided herein is a method of treating a disease associated with the CNS of a subject, wherein the method comprises administering a recombinant adeno-associated virus (rAAV) vector to the CNS of the subject, wherein the rAAV vector comprises a variant AAV capsid protein encapsidating a transgene, wherein the variant AAV capsid protein comprises an amino acid sequence that is at least 95% identical to any one of SEQ ID NOs: 10 and 12-22.

In some aspects, the variant AAV capsid protein comprises an amino acid sequence that is identical to any one of SEQ ID NOs: 10 and 12-22. In some aspects, the variant AAV capsid protein comprises an amino acid sequence that is identical to SEQ ID NO: 10. In some aspects, the variant AAV capsid protein comprises an amino acid sequence that is identical to SEQ ID NO: 12.

In one aspect provided herein is an isolated nucleic acid comprising a nucleotide sequence encoding a variant adeno-associated virus (AAV) capsid protein, wherein the variant AAV capsid protein comprises an amino acid sequence that is at least 95% identical to any one of SEQ ID NOs: 10 and 12-22.

In some aspects, the variant AAV capsid protein comprises an amino acid sequence that is identical to any one of SEQ ID NOs: 10 and 12-22. In some aspects, the variant AAV capsid protein comprises an amino acid sequence that is identical to SEQ ID NO: 10. In some aspects, the variant AAV capsid protein comprises an amino acid sequence that is identical to SEQ ID NO: 12.

In one aspect provided herein is an isolated nucleic acid comprising a nucleotide sequence of SEQ ID NO: 9 or 11. In one aspect provided herein is an isolated nucleic acid comprising a nucleotide sequence of SEQ ID NO: 9. In one aspect provided herein is an isolated nucleic acid comprising a nucleotide sequence of SEQ ID NO: 11.

In one aspect provided herein is a cell comprising an rAAV vector.

In one aspect provided herein is a plasmid comprising a nucleotide sequence of SEQ ID NO: 9 or 11. In one aspect provided herein is plasmid comprising a nucleotide sequence of SEQ ID NO: 9. In one aspect provided herein is plasmid comprising a nucleotide sequence of SEQ ID NO: 11.

### 4. BRIEF DESCRIPTION OF THE FIGURES

The foregoing and other objects, features and advantages will be apparent from the following description of particular embodiments of the invention, as illustrated in the accompanying drawings. The drawings are not necessarily to scale, emphasis instead being placed upon illustrating the principles of various embodiments of the invention.

FIG. 1 shows high packaging efficiency in terms of titer, expressed as genome copies per mL (GC/mL) of wild type AAV9 and ten (10) candidate rAAV9 vectors as provided in Table 2. Error bars represent standard error of the mean.

FIG. 2 shows the expression of GFP from AAV9 comprising SEQ ID NO: 23 (vector 2070/1686 from Table 2).

FIG. 3 shows expression of GFP using vectors 2078-2086 from Table 2 and wild type AAV 9. Vectors 2082 (AAV9 comprising SAKPAPPKPE after Q588) and 2085 (AAV9 comprising SAKPAPPKPE after S454) were found to behave similarly to WTAAV9 in cultured cells.

FIG. 4 shows higher GFP expressing cells present throughout the retina of mice injected IVT with 2082 (AAV9 comprising SAKPAPPKPE after Q588) and 2085 (AAV9 comprising SAKPAPPKPE after S454), compared to the reference AAV2-variant and WTAAV9.

FIG. 5 shows representative images from the RPE layer of mice injected IVT with 2082 (AAV9 comprising SAKPAPPKPE after Q588) and 2085 (AAV9 comprising SAKPAPPKPE after S454) as compared to images from the RPE layer of mice injected with AAV2-variant or WTAAV9.

FIG. 6 shows a graph showing GFP expression in the retina, RPE, choroid, sclera, and anterior segment after one of: formulation buffer, no injection control, AAV2 variant, AAV9.2082, AAV9.2085, and AAV9 are intravitreally administered to mice.

FIG. 7 shows Optical Coherence Tomography (OCT) indicating integrity of ocular structures 1 to 3 weeks post IVT injection at 2×10⁹ GC/eye of AAV2 variant, AAV9.2082, AAV9.2085, and AAV9.

FIGS. 8A-8B FIG. 8A shows confocal tile-scans of retinal cross-sections immunolabelled for GFP and DAPI. FIG. 8B shows immunolabelled cross-sections of mouse retina infected with AAV2 variant, AAV9.2082, AAV9.2085, and AAV9 with a focus on the retinal pigment epithelium (RPE), outer nuclear layer (ONL), and inner nuclear layer (INL), stained for GFP, RPE65, Rhodopsin, and DAPI, 3 weeks post IVT injection at 2×10⁹ GC/eye.

### 5. DETAILED DESCRIPTION

Described herein are recombinant adeno-associated viruses (rAAVs) comprising a variant AAV capsid protein comprising a heterologous amino acid sequence (or a peptide insert), wherein the variant AAV capsids and heterologous amino acid sequences are described in Section 5.1.1. In some embodiments, such rAAVs further comprise a transgene (refer to Section 5.1.4) and have enhanced functional properties (e.g., enhanced tropism, transduction, and/or homogeneous expression of the transgene) as compared to a reference AAV (e.g., an AAV that does not comprise a variant AAV capsid of the disclosure), as described in Section 5.1.2. In some embodiments, these rAAVs can be used to treat a disease or disorder in a subject. In some embodiments, an rAAV of the disclosure or compositions comprising the same can be used to treat or prevent a disease or disorder associated with the eye as described in Section 5.4. Also provided herein are rAAV vectors as described in Section 5.1.3, and pharmaceutical compositions comprising the rAAVs of the disclosure, such as described in Section 5.1.5. Also provided herein are methods of manufacturing the rAAVs of the disclosure, such as described in Section 5.2 and gene therapy as described in Section 5.3. Combination therapies are described in Section 5.5. Disease markers and methods to assess clinical outcomes are described in Section 5.6. Non-limiting illustrative examples are provided in Section 6.

5.1 Recombinant Adeno-Associated Viruses (rAAVs) and Viral Vectors

### 5.1.1 Variant AAV Capsids

Provided herein is a recombinant adeno-associated virus (rAAV) comprising a capsid protein engineered to comprise a heterologous amino acid sequence (or a peptide insert). In some embodiments a heterologous amino acid sequence is inserted (i.e., between two amino acids without deleting any capsid amino acids) into at least one of: GBS, GH loop. VR-I, VR-II, VR-III, VR-IV, VR-V, VR-VI, VR-VII, VR-VIII or VR-IX region of a capsid protein of an AAV (e.g., AAV9). In some embodiments, a heterologous amino acid sequence is inserted into the VR-I of a capsid protein of an AAV (e.g., AAV9). In some embodiments, a heterologous amino acid sequence is inserted into the VR-II of a capsid protein of an AAV (e.g., AAV9). In some embodiments, a heterologous amino acid sequence is inserted into the VR-III of a capsid protein of an AAV (e.g., AAV9). In some embodiments, a heterologous amino acid sequence is inserted into the VR-IV of a capsid protein of an AAV (e.g., AAV9). In some embodiments, a heterologous amino acid sequence is inserted into the VR-V of a capsid protein of an AAV (e.g., AAV9). In some embodiments, a heterologous amino acid sequence is inserted into the VR-VI of a capsid protein of an AAV (e.g., AAV9). In some embodiments, a heterologous amino acid sequence is inserted into the VR-VII of a capsid protein of an AAV (e.g., AAV9). In some embodiments, a heterologous amino acid sequence is inserted into the VR-VIII of a capsid protein of an AAV (e.g., AAV9). In some embodiments, a heterologous amino acid sequence is inserted into the VR-IX of a capsid protein of an AAV (e.g., AAV9). In some embodiments, VR-I is from amino acid position 262-269 or 262-270 of AAV9 or a corresponding position in another AAV (e.g., refer to corresponding position in Table 1 below). In some embodiments, VR-II is from amino acid position 327-332 of AAV9 or a corresponding position in another AAV. In some embodiments, VR-III is from amino acid position 382-386 of AAV9 or a corresponding position in another AAV. In some embodiments, VR-IV is from amino acid position 452-460 of AAV9 or a corresponding position in another AAV. In some embodiments, GBS is from amino acid position 465-476 of AAV9 or a corresponding position in another AAV. In some embodiments, GH loop is from amino acid position 448-602 of AAV9 or a corresponding position in another AAV. In some embodiments, VR-V is from amino acid position 488-505 of AAV9 or a corresponding position in another AAV. In some embodiments, VR-VI is from amino acid position 527-539 of AAV9 or a corresponding position in another AAV. In some embodiments, VR-VII is from amino acid position 545-558 of AAV9 or a corresponding position in another AAV. In some embodiments, VR-VIII is from amino acid position 581-593 of AAV9 or a corresponding position in another AAV. In some embodiments, VR-IX is from amino acid position 704-714 or 704-712 of AAV9 or a corresponding position in another AAV.

In some embodiments, provided herein is an engineered capsid protein comprising a peptide insertion (or an insertion of a heterologous amino acid sequence) of about 4 to about 35 (e.g., 7, 8, 9, 10, 11, 12, 13, 14, or 15) contiguous amino acids (e.g., from a heterologous protein), within or near a variable region (e.g., VR-VIII, VR-IV, and/or VR-VI of AAV9 or a corresponding variable region in another AAV) of a virus capsid. For example, the amino acids in each variable region of various AAV serotypes are provided in Table 1 below. In some embodiments, the peptide insertion is between two amino acids without deleting any capsid amino acid(s) of an AAV capsid (e.g., AAV9). In some embodiments, the peptide insertion occurs within (i.e., between two amino acids without deleting any capsid amino acids) variable region VIII (VR-VIII), VR-IV, VR-VI of an AAV9 capsid, or a corresponding region in another AAV serotype (e.g., as provided in Table 1 below). In some embodiments, the peptide insertion is from a heterologous protein or domain that is not an AAV capsid protein or domain. In some embodiments, provided herein are recombinant capsid proteins, and rAAVs comprising the same, that have inserted peptides that target specific tissues and/or promote rAAV cellular uptake, transduction, and/or genome integration. In some embodiments, the engineered capsids have one or more amino acid substitution and/or deletion, e.g., that promote transduction and/or tissue tropism (e.g., associated with the eye).

TABLE 1
Examples of AAV Capsid Variable Regions

| | | | VR- | VR- | | VR- | VR- | VR- | VR- | VR- | GH |
|---|---|---|---|---|---|---|---|---|---|---|---|
| AAV | VR-I | VR-II | III | IV | GBS | V | VI | VII | VIII | IX | loop |
| AAV-2 | 262- | 325- | 380- | 450- | 464- | 487- | 526- | 544- | 580- | 703- | 446- |
| | 268 | 330 | 384 | 459 | 475 | 504 | 538 | 557 | 592 | 711 | 600 |
| AAV-4 | 256- | 316- | 372- | 444- | 459- | 481- | 526- | 542- | 579- | 702- | 440- |
| | 260 | 321 | 378 | 454 | 470 | 504 | 536 | 556 | 591 | 710 | 599 |
| AAV-5 | 252- | 316- | 372- | 443- | 452- | 474- | 513- | 531- | 570- | 692- | 439- |
| | 258 | 321 | 377 | 446 | 462 | 491 | 525 | 547 | 582 | 700 | 590 |
| AAV-6 | 262- | 326- | 381- | 451- | 465- | 488- | 527- | 545- | 581- | 704- | 447- |
| | 269 | 331 | 385 | 460 | 476 | 505 | 539 | 558 | 593 | 712 | 601 |
| AAV-7 | 263- | 327- | 382- | 452- | 467- | 490- | 529- | 547- | 582- | 705- | 448- |
| | 270 | 332 | 386 | 462 | 478 | 507 | 541 | 559 | 594 | 713 | 602 |
| AAV-8 | 263- | 328- | 383- | 453- | 467- | 490- | 529- | 548- | 583- | 706- | 449- |
| | 271 | 333 | 387 | 462 | 478 | 507 | 541 | 560 | 595 | 714 | 603 |
| AAV-9 | 262- | 327- | 382- | 452- | 465- | 488- | 527- | 545- | 581- | 704- | 448- |
| | 270 | 332 | 386 | 460 | 476 | 505 | 539 | 558 | 593 | 712 | 602 |
| AAVrh8 | 262- | 327- | 382- | 452- | 465- | 488- | 527- | 545- | 581- | 704- | 448- |
| | 270 | 332 | 386 | 460 | 476 | 505 | 539 | 558 | 593 | 712 | 602 |
| AAVrh10 | 263- | 328- | 383- | 453- | 467- | 490- | 529- | 548- | 583- | 706- | 449- |
| | 271 | 333 | 387 | 462 | 478 | 507 | 541 | 560 | 595 | 714 | 603 |

In some embodiments, a heterologous amino acid sequence (or a peptide) is inserted before or after at least one amino acid at position: 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600 of an AAV (e.g., AAV9 or a corresponding position in another AAV). In some particular embodiments, the peptide insertion occurs immediately after one of the amino acid residues within: 450-456, 451-461, 454-456, 450-460, 440-470, 430-480, 420-500, 510-600, 520-590, 520-540, 530-540, 530-550, 530-535, 532-535, 560-590, 580-590, 570-600, 585-589, 585-590 of AAV9 or a corresponding position in another AAV.

In some embodiments, a heterologous amino acid sequence is inserted between two amino acids present between amino acids: 1 and 730, 100 and 700, 200 and 650, 300 and 600, 400 and 600, 400 and 700, 500 and 600, 455 and 589, and/or 450 and 590 of a capsid protein of an AAV (e.g., AAV9 or a corresponding position in another AAV). In some embodiments, a heterologous amino acid sequence is inserted at an amino acid between amino acids: 1 and 730, 100 and 700, 200 and 650, 300 and 600, 400 and 600, 400 and 700, 500 and 600, 455 and 589, and/or 450 and 590 of a capsid protein of an AAV (e.g., AAV9 or a corresponding position in another AAV). In some embodiments, the heterologous amino acid sequence is surface exposed once introduced into the AAV. In some embodiments, “corresponding” amino acid residues in a different capsid amino acid sequences is such that a “corresponding” amino acid residue is lined up at the same position in the alignment as the residue in the reference sequence (e.g., AAV9). A heterologous amino acid sequence (or peptide insertion) described as inserted “at” a given site refers to insertion immediately before, that is having a peptide bond to the carboxy group of, the residue normally found at that site in the wild type virus. For example, insertion at position 589 or A589 in AAV9 means that the peptide insertion appears between Q588 and amino acid A589 in the AAV9 wildtype capsid protein or a corresponding position in another AAV (e.g., refer to corresponding position in Table 1 below). In some embodiments, insertion at position 533 or R533 in AAV9 means that the peptide insertion appears between D532 and amino acid R533 in the AAV9 wildtype capsid protein or a corresponding position in another AAV. In some embodiments, insertion at position 455 or G455 in AAV9 means that the peptide insertion appears between S454 and amino acid G455 in the AAV9 wildtype capsid protein or a corresponding position in another AAV. In embodiments, there is no deletion of an amino acid residue at or near (within 5, 10, 15 residues or within the structural loop that is the site of the insertion) the point of insertion. In some embodiments, a heterologous amino acid sequence (or a peptide) is inserted at position: 455, 533, and/or 589 of AAV9 or a corresponding position in another AAV (e.g., refer to corresponding position in Table 1 below). In some embodiments, a heterologous amino acid sequence or a peptide is inserted at position: 454, 532, and/or 588 of AAV9 or a corresponding position in another AAV. In some embodiments, a heterologous amino acid sequence or a peptide is inserted at position: 456, 534, and/or 590 of AAV9 or a corresponding position in another AAV. In some embodiments, a heterologous amino acid sequence (or a peptide) is inserted after position: 455, 533, and/or 589 of AAV9 or a corresponding position in another AAV. In some embodiments, a heterologous amino acid sequence (or a peptide) is inserted before position: 455, 533, and/or 589 of AAV9 or a corresponding position in another AAV. In some embodiments, a heterologous amino acid sequence (or a peptide) is inserted before at least one amino acid at position: 455, 533, and/or 589 of AAV9 or a corresponding position in another AAV. In some embodiments, a heterologous amino acid sequence (or a peptide) is inserted after at least one amino acid at position: 455, 533, and/or 589 of AAV9 or a corresponding position in another AAV.

In some embodiments, an rAAV of the disclosure comprises a variant AAV capsid protein comprising a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 7, 8, 9, 10, 11, 12, 13, 14, 15, 16, 17, 18, 19, 20, 21, 22, 23, 24, 25, 26, 27, 28, 29, 30, or 31 contiguous amino acids of SEQ ID NO: 1 (SEQ ID NO: 1 is KDEPQRRSARLSAKPAPPKPEPKPKKAPAKK). In some embodiments, a variant AAV capsid protein comprises a heterologous amino acid sequence (or a peptide insertion) comprising about or at least about or at most about: 7, 8, 9, 10, 11, 12, 13, 14, 15, 16, 17, 18, 19, 20, 21, 22, 23, 24, 25, 26, 27, 28, 29, 30, or 31 contiguous amino acids of SEQ ID NO: 1. In some embodiments, a variant AAV capsid protein comprises a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to SEQ ID NO: 1. In some embodiments, a variant AAV capsid protein comprises a heterologous amino acid sequence or a peptide insertion having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 7, 8, 9, 10, 11, 12, 13, 14, 15, 16, 17, 18, 19, 20, 21, 22, 23, 24, 25, 26, 27, 28, 29, 30, or 31 contiguous amino acids of SEQ ID NO: 1 and comprises about or at least about or at most about: 1, 2, 3, 4, 5, 6, 7, 8, 9, 10, 11, 12, 13, 14, 15, 16, 17, 18, 19, 20, or 21 amino acid substitution, deletion, mutation, and/or addition as compared to the amino acids in SEQ ID NO: 1. In some embodiments, a variant AAV capsid protein comprises a heterologous amino acid sequence (or a peptide insertion) that has about or at least about or at most about: 1, 2, 3, 4, 5, 6, 7, 8, 9, 10, 11, 12, 13, 14, 15, 16, 17, 18, 19, 20, 21, 22, 23, 24, or 25 amino acid substitution, deletion, mutation, and/or addition as compared to the amino acids in SEQ ID NO: 1. In some embodiments, a variant AAV capsid protein comprises a heterologous amino acid sequence (or a peptide insertion) comprising SEQ ID NO: 1. In some embodiments, a variant AAV capsid protein comprises a heterologous amino acid sequence (or a peptide insertion) consisting of SEQ ID NO: 1. In some embodiments, a variant AAV capsid is identical to a parental AAV capsid (e.g., AAV9) except for a heterologous amino acid sequence (or a peptide insertion). In some embodiments, at least one additional amino acid is added to either the amino terminus and/or the carboxyl terminus of a heterologous sequence (e.g., SEQ ID NO: 1). In some embodiments, a methionine is added to the beginning of SEQ ID NO: 1 to result in SEQ ID NO: 23 (MKDEPQRRSARLSAKPAPPKPEPKPKKAPAKK).

In some embodiments, an rAAV of the disclosure comprises a variant AAV capsid protein comprising a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 2, 3, 4, 5, 6, 7, 8, 9, 10, or 11 contiguous amino acids of SEQ ID NO: 2 (SEQ ID NO: 2 is KDEPQRRSARL). In some embodiments, a variant AAV capsid protein comprises a heterologous amino acid sequence (or a peptide insertion) comprising about or at least about or at most about: 2, 3, 4, 5, 6, 7, 8, 9, 10, or 11 contiguous amino acids of SEQ ID NO: 2. In some embodiments, a variant AAV capsid protein comprises a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to SEQ ID NO: 2. In some embodiments, a variant AAV capsid protein comprises a heterologous amino acid sequence or a peptide insertion having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 2, 3, 4, 5, 6, 7, 8, 9, 10, or 11 contiguous amino acids of SEQ ID NO: 2 and comprises about or at least about or at most about: 1, 2, 3, 4, 5, 6, 7, 8, or 9 amino acid substitution, deletion, mutation, and/or addition as compared to the amino acids in SEQ ID NO: 2. In some embodiments, a variant AAV capsid protein comprises a heterologous amino acid sequence (or a peptide insertion) that has about or at least about or at most about: 1, 2, 3, 4, 5, or 6 amino acid substitution, deletion, mutation, and/or addition as compared to the amino acids in SEQ ID NO: 2. In some embodiments, a variant AAV capsid protein comprises a heterologous amino acid sequence (or a peptide insertion) comprising SEQ ID NO: 2. In some embodiments, a variant AAV capsid protein comprises a heterologous amino acid sequence (or a peptide insertion) consisting of SEQ ID NO: 2. In some embodiments, a variant AAV capsid is identical to a parental AAV capsid (e.g., AAV9) except for a heterologous amino acid sequence (or a peptide insertion).

In some embodiments, an rAAV of the disclosure comprises a variant AAV capsid protein comprising a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 2, 3, 4, 5, 6, 7, 8, 9, or 10 contiguous amino acids of SEQ ID NO: 3 (SEQ ID NO: 3 is SAKPAPPKPE). In some embodiments, a variant AAV capsid protein comprises a heterologous amino acid sequence (or a peptide insertion) comprising about or at least about or at most about: 2, 3, 4, 5, 6, 7, 8, 9, or 10 contiguous amino acids of SEQ ID NO: 3. In some embodiments, a variant AAV capsid protein comprises a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to SEQ ID NO: 3. In some embodiments, a variant AAV capsid protein comprises a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 2, 3, 4, 5, 6, 7, 8, 9, or 10 contiguous amino acids of SEQ ID NO: 3 and comprises about or at least about or at most about: 1, 2, 3, 4, 5, 6, 7, or 8 amino acid substitution, deletion, mutation, and/or addition as compared to the amino acids in SEQ ID NO: 3. In some embodiments, a variant AAV capsid protein comprises a heterologous amino acid sequence (or a peptide insertion) that has about or at least about or at most about: 1, 2, 3, 4, 5, or 6 amino acid substitution, deletion, mutation, and/or addition as compared to the amino acids in SEQ ID NO: 3. In some embodiments, a variant AAV capsid protein comprises a heterologous amino acid sequence (or a peptide insertion) comprising SEQ ID NO: 3. In some embodiments, a variant AAV capsid protein comprises a heterologous amino acid sequence (or a peptide insertion) consisting of SEQ ID NO: 3. In some embodiments, a variant AAV capsid is identical to a parental AAV capsid (e.g., AAV9) except for a heterologous amino acid sequence (or a peptide insertion).

In some embodiments, an rAAV of the disclosure comprises a variant AAV capsid protein comprising a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 2, 3, 4, 5, 6, 7, 8, 9, or 10 contiguous amino acids of SEQ ID NO: 4 (SEQ ID NO: 4 is PKPKKAPAKK). In some embodiments, a variant AAV capsid protein comprises a heterologous amino acid sequence (or a peptide insertion) comprising about or at least about or at most about: 2, 3, 4, 5, 6, 7, 8, 9, or 10 contiguous amino acids of SEQ ID NO: 4. In some embodiments, a variant AAV capsid protein comprises a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to SEQ ID NO: 4. In some embodiments, a variant AAV capsid protein comprises a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 2, 3, 4, 5, 6, 7, 8, 9, or 10 contiguous amino acids of SEQ ID NO: 4 and comprises about or at least about or at most about: 1, 2, 3, 4, 5, 6, 7, or 8 amino acid substitution, deletion, mutation, and/or addition as compared to the amino acids in SEQ ID NO: 4. In some embodiments, a variant AAV capsid protein comprises a heterologous amino acid sequence (or a peptide insertion) that has about or at least about or at most about: 1, 2, 3, 4, 5, or 6 amino acid substitution, deletion, mutation, and/or addition as compared to the amino acids in SEQ ID NO: 4. In some embodiments, a variant AAV capsid protein comprises a heterologous amino acid sequence (or a peptide insertion) comprising SEQ ID NO: 4. In some embodiments, a variant AAV capsid protein comprises a heterologous amino acid sequence (or a peptide insertion) consisting of SEQ ID NO: 4.

In some embodiments, an rAAV of the disclosure comprises a variant AAV capsid protein comprising a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 2, 3, 4, 5, 6, 7, 8, 9, or 10 contiguous amino acids of SEQ ID NO: 23 (SEQ ID NO: 23 is

MKDEPQRRSARLSAKPAPPKPEPKPKKAPAKK). In some embodiments, a variant AAV capsid protein comprises a heterologous amino acid sequence (or a peptide insertion) comprising about or at least about or at most about: 2, 3, 4, 5, 6, 7, 8, 9, or 10 contiguous amino acids of SEQ ID NO: 23. In some embodiments, a variant AAV capsid protein comprises a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to SEQ ID NO: 23. In some embodiments, a variant AAV capsid protein comprises a heterologous amino acid sequence (or a peptide insertion) having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, 99%, or 100% sequence identity to about or at least about or at most about: 2, 3, 4, 5, 6, 7, 8, 9, or 10 contiguous amino acids of SEQ ID NO: 23 and comprises about or at least about or at most about: 1, 2, 3, 4, 5, 6, 7, or 8 amino acid substitution, deletion, mutation, and/or addition as compared to the amino acids in SEQ ID NO: 23. In some embodiments, a variant AAV capsid protein comprises a heterologous amino acid sequence (or a peptide insertion) that has about or at least about or at most about: 1, 2, 3, 4, 5, or 6 amino acid substitution, deletion, mutation, and/or addition as compared to the amino acids in SEQ ID NO: 23. In some embodiments, a variant AAV capsid protein comprises a heterologous amino acid sequence (or a peptide insertion) comprising SEQ ID NO: 23. In some embodiments, a variant AAV capsid protein comprises a heterologous amino acid sequence (or a peptide insertion) consisting of SEQ ID NO: 23.

In some embodiments, a variant AAV capsid protein comprises a heterologous amino acid sequence (or a peptide insertion) comprising at least one of: SEQ ID NO: 1, SEQ ID NO:2, SEQ ID NO: 3, SEQ ID NO: 4, and/or SEQ ID NO: 23. In some embodiments, a variant AAV capsid protein comprises a heterologous amino acid sequence (or a peptide insertion) consisting of at least one of: SEQ ID NO: 1, SEQ ID NO:2, SEQ ID NO: 3, SEQ ID NO: 4, and/or SEQ ID NO: 23. In some embodiments, a variant AAV capsid is identical to a parental AAV capsid (e.g., AAV9) except for a heterologous amino acid sequence or a peptide insertion. In some embodiments, a variant AAV capsid comprises more than one peptide insertion at one or more different location(s) or variable region(s).

In some embodiments, mutations (e.g., insertions, deletions and/or substitutions), or a heterologous amino acid sequence (or a peptide insertion) is/are introduced into a DNA sequence encoding an exposed loop in the capsid protein. In some embodiments, a heterologous amino acid sequence (or a peptide) is inserted into exposed loops (e.g. hypervariable regions) in the AAV capsid. In some embodiments, a heterologous amino acid sequence (or a peptide) comprises about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more amino acids. In some embodiments, a heterologous amino acid sequence (or a peptide) comprises or consists of about 3, 4, 5, 6, 7, 8, 9, 10, 11, 12, 13, 14, 15, 16, 17, 18, 19, 20, 21, 22, 23, 24, 25, 26, 27, 28, 29, 30, or 31 contiguous amino acids of SEQ ID NO: 1. In some embodiments, a heterologous amino acid sequence (or a peptide) comprises or consists of about 3, 4, 5, 6, 7, 8, 9, 10, or 11 contiguous amino acids of SEQ ID NO: 2. In some embodiments, a heterologous amino acid sequence (or a peptide) comprises or consists of about 3, 4, 5, 6, 7, 8, 9, or 10 contiguous amino acids of SEQ ID NO: 3. In some embodiments, a heterologous amino acid sequence (or a peptide) comprises or consists of about 3, 4, 5, 6, 7, 8, 9, or 10 contiguous amino acids of SEQ ID NO: 4. In some embodiments, a heterologous amino acid sequence (or a peptide) comprises or consists of about 3, 4, 5, 6, 7, 8, 9, or 10 contiguous amino acids of SEQ ID NO: 23.

In some embodiments, a heterologous amino acid sequence (e.g., SEQ ID NO: 1, SEQ ID NO:2, SEQ ID NO: 3, SEQ ID NO: 4, and/or SEQ ID NO: 23, or about or at least about or at most about: 4, 5, 6, 7, 8, 9, 10, or more than 10 contiguous amino acids of: SEQ ID NO: 1, SEQ ID NO:2, SEQ ID NO: 3, SEQ ID NO: 4, and/or SEQ ID NO: 23) is inserted at position 455, immediately before position 455 (i.e., position 454), or immediately after position 455 (i.e., position 456) of AAV9 or a corresponding position in another AAV. In some embodiments, a heterologous amino acid sequence (e.g., SEQ ID NO: 1, SEQ ID NO:2, SEQ ID NO: 3, SEQ ID NO: 4, and/or SEQ ID NO: 23, or about or at least about or at most about: 4, 5, 6, 7, 8, 9, 10, or more than 10 contiguous amino acids of: SEQ ID NO: 1, SEQ ID NO:2, SEQ ID NO: 3, SEQ ID NO: 4, and/or SEQ ID NO: 23) is inserted at position 533, immediately before position 533, or immediately after position 533 of AAV9 or a corresponding position in another AAV. In some embodiments, a heterologous amino acid sequence (e.g., SEQ ID NO: 1, SEQ ID NO:2, SEQ ID NO: 3, SEQ ID NO: 4, and/or SEQ ID NO: 23, or about or at least about or at most about: 4, 5, 6, 7, 8, 9, 10, or more than 10 contiguous amino acids of: SEQ ID NO: 1, SEQ ID NO:2, SEQ ID NO: 3, SEQ ID NO: 4, and/or SEQ ID NO: 23) is inserted at position 589, immediately before position 589, or immediately after position 589 of AAV9 or a corresponding position in another AAV. In some embodiments, a heterologous amino acid sequence (e.g., SEQ ID NO: 1, SEQ ID NO: 2, SEQ ID NO: 3, SEQ ID NO: 4, and/or SEQ ID NO: 23, or about or at least about or at most about: 4, 5, 6, 7, 8, 9, 10, or more than 10 contiguous amino acids of: SEQ ID NO: 1, SEQ ID NO: 2, SEQ ID NO: 3, SEQ ID NO: 4, and/or SEQ ID NO: 23) is inserted at position 455, immediately before position 455, or immediately after position 455; and/or a heterologous amino acid sequence (e.g., SEQ ID NO: 1, SEQ ID NO:2, SEQ ID NO: 3, SEQ ID NO: 4, and/or SEQ ID NO: 23, or about or at least about or at most about: 4, 5, 6, 7, 8, 9, 10, or more than 10 contiguous amino acids of: SEQ ID NO: 1, SEQ ID NO:2, SEQ ID NO: 3, SEQ ID NO: 4, and/or SEQ ID NO: 23) is inserted at position 533, immediately before position 533, or immediately after position 533; and/or a heterologous amino acid sequence (e.g., SEQ ID NO: 1, SEQ ID NO:2, SEQ ID NO: 3, SEQ ID NO: 4, and/or SEQ ID NO: 23, or about or at least about or at most about: 4, 5, 6, 7, 8, 9, 10, or more than 10 contiguous amino acids of: SEQ ID NO: 1, SEQ ID NO:2, SEQ ID NO: 3, SEQ ID NO: 4, and/or SEQ ID NO: 23) is inserted at position 589, immediately before position 589, or immediately after position 589 of AAV9 or a corresponding position in another AAV.

In some embodiments, a heterologous amino acid sequence (e.g., SEQ ID NO: 1, SEQ ID NO:2, SEQ ID NO: 3, SEQ ID NO: 4, and/or SEQ ID NO: 23, or about or at least about or at most about: 4, 5, 6, 7, 8, 9, 10, or more than 10 contiguous amino acids of: SEQ ID NO: 1, SEQ ID NO:2, SEQ ID NO: 3, SEQ ID NO: 4, and/or SEQ ID NO: 23) is inserted between S454 and G455, D532 and R533, and/or Q588 and A589 of AAV9 or a corresponding position in another AAV. In some embodiments, a cysteine is added at the N-terminus of a heterologous amino acid sequence. In some embodiments, a cysteine residue is added to the N-terminus, C-terminus, or both of a heterologous amino acid sequence. In some embodiments, the capsid is chosen and/or further modified to reduce recognition of the AAV particles by the subject's immune system, such as avoiding pre-existing antibodies in the subject. In some embodiments, the capsid is chosen and/or further modified to enhance desired tropism/targeting related to a specific location

In some embodiments, the HI loop is targeted (mutated) while in others, the DE loop is targeted (mutated). In some embodiments, a mutation (e.g., insertion, deletion and/or substitution) and/or a heterologous amino acid sequence (or a peptide) is/are introduced in both loops. In some embodiments, a mutation (e.g., insertion, deletion and/or substitution) and/or a heterologous amino acid sequence is/are introduced into the VR region of a surface loop, including into VR-I, VR-II, VR-III, VR-IV, VR-V, VR-VI, VR-VII, VR-VIII and or VR-IX. In some embodiments, a mutation (e.g., insertion, deletion, and/or substitution) and/or a heterologous amino acid sequence is/are introduced in VR-IV, VR-VI and/or VR-VIII. In some embodiments, a mutation (e.g., insertion, deletion, and/or substitution) and/or a heterologous amino acid sequence is/are introduced into the AAV capsid proteins VP1, VP2 or VP3, or in two of the capsid proteins in any combination, or in all three. In some embodiments, a mutation and/or a heterologous amino acid sequence is/are introduced into VP1. In some embodiments, a mutation and/or a heterologous amino acid sequence is/are introduced into VP2. In some embodiments, a mutation and/or a heterologous amino acid sequence is/are introduced into VP3. In some embodiments, a mutation and/or a heterologous amino acid sequence is/are introduced into VP1 and VP2. In some embodiments, a mutation and/or a heterologous amino acid sequence is/are introduced into VP1 and VP3. In some embodiments, a mutation and/or a heterologous amino acid sequence is/are introduced into VP2 and VP3. In some embodiments, a mutation and/or a heterologous amino acid sequence is/are introduced into VP1, VP2, and VP3. In some embodiments, a single mutation (e.g., an insertion, a deletion and/or a substitution) is introduced at a single site in a gene encoding a capsid protein, while in other embodiments, more than about, at least about, or at most about: 1, 2, 3, 4, 5, 6, 7, 10, 20, 30, 40, 50, 100 or more (including any number between 1 and 100 or more) mutations (e.g., insertions, deletions and/or substitutions) are introduced in a gene encoding a capsid protein.

In some embodiments, an amino acid substitution is a conservative substitution. Illustrative examples for conserved amino acid exchanges are amino acid substitutions that maintain structural and/or functional properties of the amino acids' side-chains, e.g., an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In some embodiments, a conservative amino acid substitution is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In contrast, examples of non-conserved amino acid exchanges are amino acid substitutions that do not maintain structural and/or functional properties of the amino acids' side-chains, e.g., an aromatic amino acid is substituted for a basic, acidic, or aliphatic amino acid, an acidic amino acid is substituted for an aromatic, basic, or aliphatic amino acid, a basic amino acid is substituted for an acidic, aromatic or aliphatic amino acid, and an aliphatic amino acid is substituted for an aromatic, acidic or basic amino acid.

In some embodiments, a heterologous amino acid sequence is inserted at one of more of a VR-I site, VR-II site, VR-III site, VR-IV site, VR-V site, VR-VI site, VR-VII site, VR-VIII site, and/or VR-IX site of a parental AAV capsid (e.g., AAV9). In some embodiments, the variant AAV capsid protein comprises a sequence that shares about or at least about 80%, 85%, 90%, 95%, 96%, 97%, 98%, 99%, 99.5%, or 100% identity to a parental AAV or a naturally found AAV (e.g., AAV9 or AAV8). In some embodiments, the variant AAV capsid protein comprises a sequence that shares about or at least about 80%, 85%, 90%, 95%, 96%, 97%, 98%, 99%, 99.5%, or 100% identity to SEQ ID NO: 5. In some embodiments, the variant AAV capsid protein comprises a sequence that shares about or at least about 80%, 85%, 90%, 95%, 96%, 97%, 98%, 99%, 99.5%, or 100% identity to SEQ ID NO: 5, excluding the VR-I site, VR-II site, VR-III site, VR-IV site, VR-V site, VR-VI site, VR-VII site, VR-VIII site, and/or VR-IX site. In some embodiments, the variant AAV capsid protein comprises a sequence that is identical to SEQ ID NO: 5 except for a heterologous amino acid sequence of the disclosure. In some embodiments, the variant AAV capsid protein comprises a sequence that shares about or at least about 80%, 85%, 90%, 95%, 96%, 97%, 98%, 99%, 99.5%, or 100% identity to SEQ ID NO: 7. In some embodiments, the variant AAV capsid protein comprises a sequence that shares about or at least about 80%, 85%, 90%, 95%, 96%, 97%, 98%, 99%, 99.5%, or 100% identity to SEQ ID NO: 7, excluding the VR-I site, VR-II site, VR-III site, VR-IV site, VR-V site, VR-VI site, VR-VII site, VR-VIII site, and/or VR-IX site. In some embodiments, the variant AAV capsid protein comprises a sequence that is identical to SEQ ID NO: 7 except for a heterologous amino acid sequence of the disclosure.

In some embodiments, provided herein are AAV9 vectors comprising a viral genome comprising an expression cassette for expression of the therapeutic product, under the control of regulatory elements and flanked by ITRs and a viral capsid that has the amino acid sequence of the AAV9 capsid protein or is about or at least about 95%, 96%, 97%, 98%, 99% or 99.9% identical to the amino acid sequence of the AAV9 capsid protein (SEQ ID NO: 5) while retaining the biological function of the AAV9 capsid. In certain embodiments, the encoded AAV9 capsid has the sequence of SEQ ID NO: 5 with about, at least about, or at most about: 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV9 capsid.

In some embodiments, the variant AAV capsid protein comprises a sequence that shares about or at least about 80%, 85%, 90%, 95%, 96%, 97%, 98%, 99%, 99.5%, or 100% identity to any one of SEQ ID NOs: 10 and 12-22. In certain embodiments, the encoded AAV9 capsid has the sequence of any one of SEQ ID NOs: 10, and 12-22 with about, at least about, or at most about: 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV9 capsid. In certain embodiments, the variant AAV capsid protein has the sequence of any one of SEQ ID NOs: 10, and 12-22.

In some embodiments, an AAV of the disclosure can be an AAV derived from a naturally occurring “wild-type” virus, an AAV derived from an rAAV genome packaged into a capsid comprising capsid proteins encoded by a naturally occurring cap gene and/or from an rAAV genome packaged into a capsid comprising capsid proteins encoded by a non-naturally occurring capsid cap gene. An example of the latter includes an rAAV having a capsid protein comprising a peptide insertion into the amino acid sequence of the naturally-occurring capsid. In some embodiments, the rAAV of the disclosure comprises a peptide insertion in a variable region (e.g., VR-VIII, VR-IV, VR-VI) and can also comprise additional amino acid substitutions or deletions. In some embodiments, the capsid protein is from at least one AAV type selected from AAV serotype 1 (AAV1), serotype 2 (AAV2), AAV2tYF, serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 10 (AAV10), serotype 11 (AAV11), serotype 12 (AAV12), serotype 13 (AAV13), AAV10, serotype rh10 (AAVrh10), AAV11, AAV12, AAV13, serotype rh20 (AAVrh20), serotype rh39 (AAVrh39), serotype hu.37 (AAVhu.37), and serotype rh74 (AAVrh74). In some embodiments, the capsid protein is from an AAV9.

In some embodiments, an AAV9 capsid is characterized by DNAse-resistant particle which is an assembly of about 60 variable proteins (vp) which are typically expressed as alternative splice variants resulting in proteins of different length of SEQ ID NO: 5. See also Genbank Accession No. AAS99264.1, which is incorporated herein by reference. See, also U.S. Pat. No. 7,906,111 and WO 2005/033321. In some embodiments, an AAV9 variant includes those described in, e.g., WO2016/049230, U.S. Pat. No. 8,927,514, US 2015/0344911, and U.S. Pat. No. 8,734,809. The amino acid sequence is reproduced in SEQ ID NO: 5 and the coding sequence is reproduced in SEQ ID NO: 6. In one embodiment, the AAV9 capsid includes a capsid encoded by SEQ ID NO: 6, or a sequence sharing about or at least about 90%, 95%, 95%, 98% or 99% identity therewith.

The largest protein, vp1, is generally the full-length of the amino acid sequence of SEQ ID NO: 5 (aa 1-736 of SEQ ID NO: 5). In certain embodiments, the AAV9 vp2 protein has the amino acid sequence of 138 to 736 of SEQ ID NO: 5. In certain embodiments, the AAV9 vp3 has the amino acid sequence of 203 to 736 of SEQ ID NO: 5. In certain embodiments, the vp 1, 2 or 3 proteins may have truncations (e.g., 1 or more amino acids at the N-terminus or C-terminus). An AAV9 capsid is composed of about 60 vp proteins, in which vp1, vp2 and vp3 are present in a ratio of about 1 vp, to about 1 vp2, to about 10 to 20 vp3 proteins within the assembled capsid. This ratio may vary depending upon the production system used. In certain embodiments, an engineered AAV9 capsid may be generated in which vp2 is absent.

In certain embodiments, AAV variant capsids that can be used according to the invention described herein include Anc80 or Anc80L65, as described in Zinn et al., 2015, Cell Rep. 12 (6): 1056-1068, which is incorporated by reference in its entirety. In certain embodiments, an AAV variant capsid that can be used according to the disclosure comprises one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In certain embodiments, an AAV2 variant capsid that can be used according to the disclosure include AAV.7m8 (SEQ ID NO:24), as described in U.S. Pat. Nos. 9,193,956; 9,458,517; and 9,587,282 and US patent application publication no. 2016/0376323, each of which is incorporated herein by reference in its entirety. In certain embodiments, an AAV variant capsid that can be used according to the disclosure include any AAV disclosed in U.S. Pat. No. 9,585,971, such as AAV-PHP.B. In particular embodiments, the AAV for use in compositions and methods provided herein is an AAV disclosed in any of the following: U.S. Pat. Nos. 7,282,199; 7,906,111; 8,524,446; 8,906,675; 8,999,678; 8,628,966; 8,927,514; 8,734,809; 9,284,357; 9,409,953; 9,169,299; 9,193,956; 9,458,517; 9,587,282; 9,737,618; 9,840,719; US patent application publication nos. 2015/0374803; 2015/0126588; 2017/0067908; 2013/0224836; 2016/0215024; 2017/0051257; and International Patent Application Nos. PCT/US2002/033630; PCT/US2004/028817; PCT/2002/033629; PCT/US2006/013375; PCT/US2015/034799; PCT/EP2015/053335; PCT/US2016/042472; PCT/US2017/027392 (each of which is incorporated herein by reference in its entirety).

Also provided are nucleic acids encoding an engineered capsid protein and variants thereof, packaging cells for expressing the nucleic acids to produce an rAAV vector, an rAAV vector further comprising a therapeutic product (e.g., transgene), and pharmaceutical compositions comprising an rAAV vector; as well as methods of using an rAAV vector to deliver a therapeutic protein to a cell or target tissue of a subject in need thereof. In some embodiments, the nucleotide sequence that encodes a variant AAV capsid protein comprises a sequence that shares about or at least about 80%, 85%, 90%, 95%, 96%, 97%, 98%, 99%, 99.5%, or 100% identity to SEQ ID NO: 9 or 11. In some embodiments, the nucleotide sequence that encodes a variant AAV capsid protein comprises a sequence that is identical to SEQ ID NO: 9. In some embodiments, the nucleotide sequence that encodes a variant AAV capsid protein comprises a sequence that is identical to SEQ ID NO: 11. In some embodiments, the nucleotide sequence that encodes a variant AAV capsid protein comprises the nucleotide sequence of SEQ ID NO: 8.

It is within the skill in the art to design nucleic acid sequences encoding an AAV9 capsid, including DNA (genomic or cDNA), or RNA (e.g., mRNA). In certain embodiments, the nucleic acid sequence encoding the AAV9 vp1 capsid protein is provided in SEQ ID NO: 6. In other embodiments, a nucleic acid sequence of 70% to 99.9% identity to SEQ ID NO: 6 may be selected to express the AAV9 capsid. In certain other embodiments, the nucleic acid sequence is at least about 75% identical, at least 80% identical, at least 85%, at least 90%, at least 95%, at least 97% identical, or at least 99% to 99.9% identical to SEQ ID NO: 6.

In some embodiments, an rAAV comprising the variant AAV capsid of the disclosure (e.g., an AAV capsid comprising a heterologous amino acid sequence of the disclosure) has enhanced cell or tissue tropism (e.g., cell or tissue associated with the eye) as compared to an AAV comprising an unmodified capsid or a capsid without a heterologous amino acid sequence of the disclosure; and/or is homogeneously expressed or uniformly distributed at a desired location (e.g, homogeneously expressed or uniformly widespread at the retina or an area within the eye of a subject) after the rAAV is administered to a subject (refer to Section 5.1.2).

5.1.2 Properties of rAAV Comprising a Variant AAV Capsid

Provided herein are recombinant adeno-associated viruses (rAAVs) comprising a variant AAV capsid as described in Section 5.1.1, wherein a variant AAV capsid of the disclosure provides for at least one enhanced property (e.g., enhanced cell or tissue tropism, transduction, and/or homogeneous distribution of a transgene at a specific location after such rAAVs are administered to a subject) as compared to a wild-type AAV or an AAV that does not comprise a variant AAV capsid of the disclosure. In some embodiments, a variant capsid comprising a heterologous amino acid sequence as described in Section 5.1.1 promotes transduction and/or tissue tropism as described herein. In some embodiments, rAAV vectors comprising a variant capsid of the disclosure enhances targeted delivery, improves transduction and/or treatment of disorders associated with the target tissue as compared to a wild-type AAV vector or an AAV vector that does not comprise a variant capsid of the disclosure. In some embodiments, an rAAV vector comprising a variant capsid comprising a heterologous amino acid sequence of the disclosure targets a tissue or cell associated with the eye. In some embodiments, the rAAV comprising a variant capsid of the disclosure facilitates delivery of therapeutic agents or transgenes for treating diseases or disorders of the eye, e.g., the retina. In some embodiments, a heterologous amino acid sequence (or peptide insertion) increases retinal tropism, directing the rAAV to target the eye or retina of the subject, crossing the blood-eye barrier. The term “retinal cell” refers to one or more of the cell types found in or near the retina, including amacrine cells, bipolar cells, horizontal cells, Muller glial cells, photoreceptor cells (e.g., rods and cones), retinal ganglion cells (e.g., midget cells, parasol cells, bistratified cells, giant retina ganglion cells, and photosensitive ganglion cells), retinal pigmented epithelium, endothelial cells of the inner limiting membrane, and the like.

In some embodiments, the rAAV vector comprises a peptide insertion as disclosed in Section 5.1.1 for retinal cell-homing, the vector is administered by in vivo injection, such as injection directly into the eye. For example, the rAAV comprising a heterologous amino acid as described in Section 5.1.1 has enhanced retinal tropism and is injected intravitreally. In some embodiments, the rAAV for increasing retinal tropism is administered by intraocular injection, e.g., through the pars plana into the vitreous body or aqueous humor of the eye. In some embodiments, the rAAV for increasing retinal tropism is administered via peribulbar injection or subconjunctival injection. In some embodiments, an advantage of rAAV vectors with peptide insertion for retinal cell-homing, is that the subject may avoid surgery, e.g., avoiding surgery to implant the therapeutic instead delivered by injection.

Provided herein are recombinant adeno-associated viruses (rAAVs) having capsid proteins engineered to comprise a heterologous amino acid sequence that confer and/or enhance desired properties (e.g., enhanced tissue or cell specific targeting, cell-specific tropism, and/or enhanced transduction efficacy). In some embodiments, the tissue or cell specific targeting or cell-specific tropism is related to a tissue or cell associated with the eye of a subject (e.g., retinal, photoreceptor, retinal pigment epithelium (RPE) cell or tissue, or any cell or tissue associated with the eye). In some embodiments, an rAAV of the disclosure has about 5%, 10%, 15%, 20%, 25%, 30%, 35%, 40%, 45%, 50%, 55%, 60%, 65%, 70%, 75%, 80%, 85%, 90%, 95%, 99%, or 100% greater tropism for a particular cell type or tissue (e.g., retinal, photoreceptor, retinal pigment epithelium (RPE) cell or tissue, or any cell or tissue associated with the eye) as compared to a reference AAV (e.g., an AAV that does not comprise a variant capsid of the disclosure). In some embodiments, an rAAV of the disclosure has at least about 5%, 10%, 15%, 20%, 25%, 30%, 35%, 40%, 45%, 50%, 55%, 60%, 65%, 70%, 75%, 80%, 85%, 90%, 95%, 99%, or 100% greater tropism for a particular cell type or tissue (e.g., retinal, photoreceptor, retinal pigment epithelium (RPE) cell or tissue, or any cell or tissue associated with the eye) as compared to a reference AAV. In some embodiments, an rAAV of the disclosure has about 125%, 150%, 175%, 200%, 250%, 300%, 350%, 400%, or 500% greater tropism for a particular cell type or tissue (e.g., retinal, photoreceptor, retinal pigment epithelium (RPE) cell or tissue, or any cell or tissue associated with the eye) as compared to a reference AAV. In some embodiments, a particular cell type or tissue is associated with the eye of a human subject. In some embodiments, the reference AAV is a wild-type AAV9 or another wild-type AAV. In some embodiments, the reference AAV is a wild-type AAV2 or another wild-type AAV. In some embodiments, the reference AAV is any AAV that does not comprise a variant capsid of the disclosure (e.g., does not comprise a variant capsid from Section 5.1.1).

In some embodiments, provided herein is an AAV capsid comprising a heterologous amino acid sequence as disclosed in Section 5.1.1 that promote transduction and/or tissue tropism of the rAAV having the variant capsid (e.g., retinal, photoreceptor, and/or retinal pigment epithelium (RPE) cell or tissue tropism, or associated with the eye of a subject). In some embodiments, a variant capsid of the disclosure is further modified to comprise one or more amino acid substitution, deletion, or addition that promote transduction and/or tissue tropism of the rAAV (e.g., retinal, photoreceptor, and/or retinal pigment epithelium (RPE) cell or tissue tropism, or tropism associated with the eye of a subject).

In some embodiments, immunohistochemistry analysis (e.g., ocular immunohistochemistry) can be performed to determine the cellular tropism of an rAAV vector of the disclosure. In some embodiments, an rAAV vector of the disclosure expressing enhanced green fluorescent protein (eGFP) is injected into an eye of a subject or non-human subject. In some embodiments, quantification of AAV vector distribution (i.e., transduction diameter) and transduction efficiency (e.g., number of eGFP-positive cells per 100 μm on histology) is measured to determine tropism (e.g., retinal tropism) of an rAAV vector comprising a variant capsid of the disclosure. In some embodiments, transduction efficiency and inner retinal tropism is determined for eyes injected (e.g., via different administration routes) with an rAAV of the disclosure. In some embodiments, fundus photography can be obtained in vivo using a fluorescent fundus camera (Micron IV; Phoenix Laboratories, Pleasanton, CA) to evaluate the intensity of eGFP signal and its distribution relative to the injection sites. In some embodiments, the number of eGFP-positive cells within the outer nuclear layer (ONL) can be manually counted in the same panoramic images utilized to determine the transduction diameter. In some embodiments, cells are counted in panoramic images after an rAAV of the disclosure is administered to a subject. The total number of eGFP-positive cells can be divided by the transduction diameter to determine the number of positive cells per, e.g., 100 μm (Number of total eGFP+ cells/[Total transduction diameter (μm)/100]=Number of eGFP+/100 μm). EGFP-positive cells within the inner nuclear layer (INL) and the retinal nerve fiber layer or ganglion cell layer (RNFL/GCL) can also be counted and qualitatively assessed. In some embodiments, a maximum width of transduction as determined through eGFP expression within the RPE and/or neural retina can be determined, for example, by using a confocal microscopy. The greatest linear diameter of transduction (“transduction diameter”), defined as the maximum distance including any eGFP-positive cells within the RPE or neural retina can be assessed, for example, by generating panoramic images from confocal images.

In some embodiments, administration of an rAAV of the disclosure comprising a variant capsid as described in Section 5.1.1 and a transgene (or therapeutic product) to a subject, results in detectable expression levels of the transgene in a subject (e.g., at injection site, throughout the eye of the subject, in a specific area of the eye of a subject, and/or homogeneous expression of the transgene in an area of the eye (e.g., retina, or injection site, or throughout the eye)). In some embodiments, expression levels of a transgene can be monitored in the eye (e.g., the aqueous humour and/or the vitreous humour) of a subject to whom a construct of the disclosure has been administered. Transgene expression may be measured by any suitable assay known in the art, including, without limitation, Western Blotting, electrochemiluminescent (ECL) immunoassays implemented using the Meso Scale Discovery (MSD) platform, and ELISA.

In some embodiments, an rAAV of the disclosure results in higher transgene expression level in a portion of the eye of the subject after the rAAV is administered to the eye of the subject compared to the transgene expression level after a reference AAV is administered to the eye of the subject or a comparable subject. In some embodiments, a comparable subject is a subject that has been diagnosed with or is at risk of developing the same disease associated with the eye as the subject. In some embodiments, a reference AAV is an AAV virion or vector comprising the corresponding parental AAV capsid protein and the transgene (e.g., AAV9 (without a heterologous amino acid sequence of the disclosure) comprising a transgene. In some embodiments, a reference AAV is an AAV2 (with or without a heterologous amino acid sequence of the disclosure) or a variant AAV2 (with or without a heterologous amino acid sequence of the disclosure) and can comprise a transgene). In some embodiments In some embodiments, the transgene expression level is higher by about or at least about 5%, 10%, 15%, 20%, 25%, 30%, 35%, 40%, 45%, 50%, 55%, 60%, 65%, 70%, 75%, 80%, 85%, 90%, 95%, 99%, or 100%. In some embodiments, the portion of the eye is the retina. In some embodiments, the portion of the eye is retinal pigment epithelium (RPE). In some embodiments, the portion of the eye is choroid. In some embodiments, the portion of the eye is sclera. In some embodiments, the portion of the eye is the anterior segment. In some embodiments, the portion of the eye is a population of photoreceptors.

In some embodiments, the ocular structural integrity is determined. In some embodiments the ocular structural integrity is determined by optical coherence tomography (OCT). In some embodiments, the ocular structural integrity remains intact after an rAAV of the disclosure is administered to an eye of a subject. In some embodiments, the ocular structural integrity remains intact for about or at least about: 1, 2, 3, 4, 5, 6, 7, 8, 9, 10, 11, 12 weeks, or longer than 12 weeks. In some embodiments, ocular structural integrity is maintained for about or at least about 1 week after an rAAV of the disclosure is administered at a dose of about or at least about 2×10⁹ GC/eye (e.g., in mice or an equivalent dose in human). In some embodiments, ocular structural integrity is maintained for about or at least about 3 weeks after an rAAV of the disclosure is administered at a dose of about or at least about 2×10⁹ GC/eye (e.g., in mice or an equivalent dose in human). In some embodiments, ocular structural integrity is maintained for about or at least about 1 week after an rAAV of the disclosure is administered at a dose of about or at least about 1×10¹⁰ GC/eye (e.g., in mice or an equivalent dose in human). In some embodiments, ocular structural integrity is maintained for about or at least about 3 weeks after an rAAV of the disclosure is administered at a dose of about or at least about 1×10¹⁰ GC/eye (e.g., in mice or an equivalent dose in human). In some embodiments, ocular structural integrity is maintained for at least about 0-3, 1-3, 0-4, 1-4, 0-5, 1-5, 0-6, 1-6, 0-7, 1-7, 0-8, 1-8, 0-9, 1-9, 0-10, 1-10 weeks, or more than 1-10 weeks after an rAAV of the disclosure is administered to an eye of a subject.

In some embodiments, expression levels of the transgene in the eye may vary between different areas of the eye, e.g., high levels of vector DNA may be detected in the retina and choroid/RPE at the area of the bleb (temporal, due to the injection) as well as the superior, inferior and nasal quadrants, while low levels may be detected in the optic nerve, optic chiasm and occipital lobe. In some embodiments, an rAAV provided herein demonstrates limited anterograde tropism along the visual pathway.

In some embodiments, administration of an rAAV of the disclosure to a subject results in detectable (e.g., detectable using a method described herein, or a method known in the art) transgene expression levels in an area of the eye, such as, the posterior segment of the eye, such as retina and retinal pigment epithelium (RPE), the retinal space, vitreous humour, and/or the aqueous humour of the eye of the subject within about 1 hour, about 3 hours, about 5 hours, about 10 hours, about 15 hours, about 20 hours, about 24 hours, about 48 hours, about 72 hours, about 1 week, about 2 weeks, about 3 weeks, about 4 weeks, about 2 months, about 3 months, about 6 months, about 9 months, about 12 months, about 15 months, about 18 months, about 21 months, or about 24 months after administration of the rAAV to the subject, wherein the levels of the transgene expression in the area of the eye, such as, the posterior segment of the eye, such as retina and retinal pigment epithelium (RPE), the retinal space, vitreous humour, and/or the aqueous humour were undetectable prior to administration of the rAAV. In some embodiments, administration of an rAAV of the disclosure to a subject results in detectable (e.g., detectable using a method described herein, or a method known in the art) transgene expression levels in the posterior segment of the eye, such as retina and retinal pigment epithelium (RPE), the retinal space, the vitreous humour, and/or the aqueous humour of the eye of the subject within about 3 months, about 6 months, about 9 months, about 12 months, about 15 months, about 18 months, about 21 months, or about 24 months of administration of the rAAV to the subject, wherein the levels of the transgene expression in the posterior segment of the eye, such as retina and retinal pigment epithelium (RPE), the retinal space, vitreous humour, and/or the aqueous humour were undetectable prior to administration of the rAAV, and wherein the transgene expression levels in serum of the subject remain undetectable (e.g., undetectable using a method described herein, or a method known in the art). In some embodiments, an increased expression level of a transgene is detectable after an rAAV of the disclosure is administered to a subject (e.g., detectable using a method described herein, or a method known in the art) in the posterior segment of the eye, such as retina and retinal pigment epithelium (RPE), the retinal space, the vitreous humour, and/or the aqueous humour of the eye of the subject as compared an expression level of a transgene after a wild-type AAV or an AAV that does not comprise a variant capsid of the disclosure is administered to a subject. In some embodiments, a transgene is detectable or undetectable using an immunofluorescence assay. In some embodiments, a transgene is detectable or undetectable using an immunostaining assay.

In some embodiments, administration of an rAAV of the disclosure to a subject results in an increase in the transgene expression levels (e.g., an increase of about or at least about 100-fold to about 500-fold, about 500-fold to about 1000-fold, about 1000-fold to about 1500-fold, about 1500-fold to about 2000-fold, about 2000-fold to about 2500-fold, about 2500-fold to about 3000-fold, about 3000-fold to about 4000-fold, about 4000-fold to about 5000-fold, about 5000-fold to about 10,000-fold, about 10,000-fold to about 15,000-fold, about 15,000-fold to about 20,000-fold, or more than about 20,000-fold) in an area of the eye, such as, the posterior segment of the eye, such as retina and retinal pigment epithelium (RPE), the retinal space, vitreous humour, and/or the aqueous humour of the eye of the subject within about 1 hour, about 3 hours, about 5 hours, about 10 hours, about 15 hours, about 20 hours, about 24 hours, about 48 hours, about 72 hours, about 1 week, about 2 weeks, about 3 weeks, about 4 weeks, about 2 months, about 3 months, about 6 months, about 9 months, about 12 months, about 15 months, about 18 months, about 21 months, or about 24 months of administration of the rAAV to the subject compared to the transgene expression levels prior to administration of the rAAV or compared to the transgene expression levels after a reference AAV (e.g. AAV that does not comprise a variant capsid of the disclosure) is administered to a subject. In some embodiments, administration of an rAAV provided herein to a subject results in an increase in the transgene expression levels (e.g., an increase of about or at least about 100-fold to about 500-fold, about 500-fold to about 1000-fold, about 1000-fold to about 1500-fold, about 1500-fold to about 2000-fold, about 2000-fold to about 2500-fold, about 2500-fold to about 3000-fold, about 3000-fold to about 4000-fold, about 4000-fold to about 5000-fold, about 5000-fold to about 10,000-fold, about 10,000-fold to about 15,000-fold, about 15,000-fold to about 20,000-fold, or more than about 20,000-fold) in the posterior segment of the eye, such as retina and retinal pigment epithelium (RPE), the retinal space, vitreous humour, and/or the aqueous humour of the eye of the subject within about 1 hour, about 3 hours, about 5 hours, about 10 hours, about 15 hours, about 20 hours, about 24 hours, about 48 hours, about 72 hours, about 1 week, about 2 weeks, about 3 weeks, about 4 weeks, about 2 months, about 3 months, about 6 months, about 9 months, about 12 months, about 15 months, about 18 months, about 21 months, or about 24 months of administration of the rAAV to the subject compared to the transgene expression levels prior to administration of the rAAV or compared to the transgene expression levels after a reference AAV (e.g. AAV that does not comprise a variant capsid of the disclosure) is administered to a subject, wherein the transgene expression levels in serum of the subject remain about constant (e.g., remain within about 10%) compared to the transgene expression levels prior to administration.

In some embodiments, administration of an rAAV provided herein to a subject results in the transgene expression in the retina or a posterior segment of the eye of the subject, e.g., expression in the retinal pigment epithelium and/or the photoreceptor outer segments, or a proportion thereof, or expression across the entire depths of the retina. In some embodiments, the transgene is homogeneously expressed in the eye or throughout the eye of the subject. In some embodiments, the transgene is homogeneously expressed in the retina of the subject. In some embodiments, homogeneous expression is measured by detecting transgene expression level at injection site. In some embodiments, homogeneous expression is measured by detecting transgene expression level throughout an area of the eye (e.g., throughout the retina). In some embodiments, homogeneous expression is measured by detecting transgene expression level at injection site and comparing such expression level with the level of transgene expression within ¼ radius from injection site. In some embodiments, homogeneous expression is measured by detecting transgene expression level at injection site and comparing such expression level with the level of transgene expression within ⅓ radius from injection site. In some embodiments, homogeneous expression is measured by detecting transgene expression level at injection site and comparing such expression level with the level of transgene expression within ½ radius from injection site. In some embodiments, homogeneous expression is measured by detecting transgene expression level at injection site and comparing such expression level with the level of transgene expression within ⅛ radius from injection site. In some embodiments, homogeneous expression is measured by detecting transgene expression level at injection site and comparing such expression level with the level of transgene expression within 1/16 radius from injection site. In some embodiments, homogeneous expression is measured by detecting transgene expression level at injection site and comparing such expression level with the level of transgene expression within ½ radius from injection site, within ⅓ radius from injection site, within ¼ radius from injection site, within ⅛ radius from injection site, and/or within 1/16 radius from injection site. In some embodiments, homogenous expression of the transgene means that the level of expression level of the transgene at injection site is about the same as the expression level of the transgene within ½ radius from injection site, within ⅓ radius from injection site, within ¼ radius from injection site, within ⅛ radius from injection site, and/or within 1/16 radius from injection site. In some embodiments, “about the same” refers to an expression level (e.g., at injection site) that is about or at most about 1%, 2%, 3%, 4%, 5%, 6%, 7%, 8%, 9%, 10%, 11%, 12%, 13%, 14%, 15%, 16%, 17%, 18%, 19%, or 20% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is about or at most about 5% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is about or at most about 10% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is about or at most about 15% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is about or at most about 20% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is within 1 standard deviation (SD), 2SD, 3SD, or 4 SD from the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is within 1 standard deviation (SD) from the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is within 2SD from the expression level at the comparison location (e.g., within ½ radius from injection site, within ⅓ radius from injection site, within ¼ radius from injection site, within ⅛ radius from injection site, and/or within 1/16 radius from injection site).

In some embodiments, the transgene is more homogeneously expressed in the eye of a subject (e.g., retina or a posterior segment of the eye) after an rAAV of the disclosure is administered to the subject by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to after a corresponding parental AAV is administered to the same subject or a different subject (e.g., an AAV that does not comprise a variant capsid comprising a peptide insertion of the disclosure). In some embodiments, homogeneity is measured using a fluorescence assay (e.g., fluorescence resonance energy transfer (FRET)). In some embodiments, homogeneity is measured using fundus autofluorescence imaging using a Phoenix MICRON IV retinal imaging microscope. In some embodiments, transgene expression is detected and characterized by immunohistochemistry. In some embodiments, the transgene is more uniformly expressed in the eye (e.g., retina) of the subject after an rAAV of the disclosure is administered to the subject as compared to after a reference AAV is administered to the same subject or a different subject (e.g., an AAV that does not comprise a variant capsid of the disclosure). In some embodiments, the transgene is expressed in a wider area of the retina of the subject after an rAAV of the disclosure is administered to the subject as compared to after a corresponding parental AAV is administered to the same subject or a different subject (e.g., an AAV that does not comprise a variant capsid of the disclosure). In some embodiments, the rAAV of the disclosure provides a widespread transgene expression in the eye of the subject or in the retinal pigmented epithelium (RPE)/choroid and/or retina.

Transgene product levels can be measured in patient samples of the vitreous humour and/or aqueous from the anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54:1616-1624, at p. 1621 and Table 5 at p. 1623, which is incorporated by reference herein in its entirety).

5.1.3 rAAV Vectors

Provided herein are rAAV vectors comprising a variant AAV capsid as disclosed in Section 5.1.1. In some embodiments, a recombinant viral vector of the disclosure is a recombinant adeno-associated virus, e.g., AAV1, AAV2, AAV2YF, AAV3, AAV4, AAV5, AAV6, AAV7, AAV8, AAV9, AAV10, AAV11, AAV12, AAV13, AAVrh20, AAVrh39, AAVhu.37, AAVrh74, and/or AAVrh10. In some embodiments, AAV based vectors provided herein comprise capsid components from one or more of AAV1, AAV2, AAV2YF, AAV3, AAV4, AAV5, AAV6, AAV7, AAV8, AAV9, AAV10, AAV11, AAV12, AAV13, AAVrh10, AAVrh20, AAVrh39, AAVhu.37, AAVrh74, and/or AAVrh10. In some embodiments, AAV based vectors provided herein comprise components from one or more of AAV2, AAV8, AAV9, AAV10, AAV11, or AAVrh10 serotypes. In some embodiments, the AAV vector of the disclosure is a chimeric AAV. In certain embodiments, the recombinant viral vector is a hybrid vector, e.g., an AAV vector placed into a “helpless” adenoviral vector. In some embodiments, a recombinant viral vector of the disclosure is an AAV9-based viral vector. In some embodiments, the rAAV of the disclosure is useful for the treatment of a subject having, suspected of having, or experiencing a symptom associate with a disease (e.g., a human subject having, suspected of having, or experiencing a symptom associated with a disease associated with the eye), which rAAV comprises a variant AAV capsid protein (e.g., AAV9) comprising a heterologous amino acid sequence as disclosed in Section 5.1.1. In certain embodiments, the AAV-based viral vector (e.g., AAV9-based viral vector) provided herein retains tropism for a specific cell or tissue (e.g., retinal cell or a cell associated with the eye). In certain embodiments, the AAV-based vector provided herein encodes the AAV rep gene (required for replication) and/or the AAV cap gene (required for synthesis of the capsid protein). In some embodiments, the rAAV (e.g., AAV9) vector of the disclosure comprises a viral genome comprising an expression cassette for expression of a transgene or therapeutic product, under the control of regulatory elements, flanked by ITRs, and a variant capsid of the disclosure (e.g., a variant AAV9 capsid). In some embodiments, the AAV-based viral vector is a targeted vector, e.g., a vector targeted to retinal cells, retinal pigment epithelial cells, or to a cell present in the eye of a subject.

In some embodiments, an rAAV vector of the disclosure comprises a nucleic acid sequence encoding an adeno-associated virus (AAV) capsid protein comprising an amino acid sequence that is at least 90%, 91%, 92%, 93%, 94%, 95%, 96%, 97%, 98%, 99%, or 100% identical to any one of SEQ ID NOs: 10 and 12-22. In some embodiments, an rAAV vector of the disclosure comprises a nucleic acid sequence encoding an adeno-associated virus (AAV) capsid protein comprising an amino acid sequence that is identical to SEQ ID NO: 10. In some embodiments, an rAAV vector of the disclosure comprises a nucleic acid sequence encoding an adeno-associated virus (AAV) capsid protein comprising an amino acid sequence that is identical to SEQ ID NO: 12. In some embodiments, a nucleic acid sequence encoding the AAV capsid protein comprises a nucleotide sequence that is at least 90%, 91%, 92%, 93%, 94%, 95%, 96%, 97%, 98%, 99%, or 100% identical to SEQ ID NO: 9 or 11. In some embodiments, a nucleic acid sequence encoding the AAV capsid protein comprises a nucleotide sequence that is identical to SEQ ID NO: 9. In some embodiments, a nucleic acid sequence encoding the AAV capsid protein comprises a nucleotide sequence that is identical to SEQ ID NO: 11.

AAV9-based viral vectors are used in certain embodiments of the methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV9)-based viral vectors encoding a therapeutic product.

Provided in some embodiments are AAV9 vectors comprising a viral genome comprising an expression cassette for expression of a transgene, under the control of regulatory elements, and flanked by ITRs and an engineered viral capsid as described herein (refer to Section 5.1.1) or is at least 95%, 96%, 97%, 98%, 99% or 99.9% identical to the amino acid sequence of the AAV9 capsid protein (e.g., SEQ ID NO: 5), while retaining the biological function of the engineered AAV9 capsid. In certain embodiments, the encoded AAV9 capsid has the sequence of wild type AAV9, with a heterologous amino acid sequence (or peptide insertion) as described herein (e.g., in Section 5.1.1), with, in addition, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitution, addition, and/or deletion, with respect to the wild type AAV sequence and retains biological function of the AAV9 capsid. Also provided are engineered AAV vectors other than AAV9 vectors, such as engineered AAV1, AAV2, AAV3, AAV4, AAV5, AAV6, AAV7, AAV8, AAV9, AAV9e, AAVrh10, AAVrh20, AAVhu.37, AAVrh39, or AAVrh74 vectors, with a heterologous amino acid sequence (or peptide insertion) as described herein (e.g., in Section 5.1.1) and 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitution, addition, and/or deletion, relative to the wild type or unengineered sequence for that AAV type and that retains its biological function

In some embodiments, provided herein are AAV8 vectors comprising a viral genome comprising an expression cassette for expression of the therapeutic product, under the control of regulatory elements and flanked by ITRs and a viral capsid that has the amino acid sequence of the AAV8 capsid protein or is at least 95%, 96%, 97%, 98%, 99% or 99.9% identical to the amino acid sequence of the AAV8 capsid protein (SEQ ID NO: 7) while retaining the biological function of the AAV8 capsid. In certain embodiments, the encoded AAV8 capsid has the sequence of SEQ ID NO: 7 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitution, addition, and/or deletion, and retaining the biological function of the AAV8 capsid.

In certain embodiments, a single-stranded AAV (ssAAV) may be used. In certain embodiments, a self-complementary vector, e.g., scAAV, may be used (see, e.g., Wu, 2007, Human Gene Therapy, 18 (2): 171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In some embodiments, a viral vector may be replication-deficient. A “replication-defective virus” or “viral vector” refers to a synthetic or recombinant viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In some embodiments, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

Fragments of AAV may be readily utilized in a variety of vector systems and host cells. Among desirable AAV fragments are the cap proteins, including the vp1, vp2, vp3 and hypervariable regions, the rep proteins, including rep 78, rep 68, rep 52, and rep 40, and the sequences encoding these proteins. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. An artificial AAV serotype may be, without limitation, a chimeric AAV capsid, a recombinant AAV capsid, or a “humanized” AAV capsid. In one embodiment, a vector contains AAV9 cap and/or rep sequences. See, U.S. Pat. No. 7,906,111, which is incorporated by reference herein.

### A. Promoters and Modifiers of Gene Expression

In certain embodiments, the recombinant vectors provided herein comprise components that modulate delivery or expression of the therapeutic product (e.g., “expression control elements”). In certain embodiments, the recombinant vectors provided herein comprise components that modulate expression of the transgene or therapeutic product. In certain embodiments, the recombinant vectors provided herein comprise components that influence binding or targeting to cells. In certain embodiments, the recombinant vectors provided herein comprise components that influence the localization of the polynucleotide encoding the therapeutic product within the cell after uptake. In certain embodiments, the recombinant vectors provided herein comprise components that can be used as detectable or selectable markers, e.g., to detect or select for cells that have taken up the polynucleotide encoding the therapeutic product.

In certain embodiments, the recombinant vectors provided herein comprise one or more promoters. In certain embodiments, the promoter is a constitutive promoter. In certain embodiments, the promoter is an inducible promoter. Inducible promoters may be preferred so that expression of the therapeutic product may be turned on and off as desired for therapeutic efficacy. Such promoters include, for example, hypoxia-induced promoters and drug inducible promoters, such as promoters induced by rapamycin and related agents. Hypoxia-inducible promoters include promoters with HIF binding sites, see, for example, Schödel, et al., 2011, Blood 117 (23): e207-e217 and Kenneth and Rocha, 2008, Biochem J. 414:19-29, each of which is incorporated by reference for teachings of hypoxia-inducible promoters. In addition, hypoxia-inducible promoters that may be used in the constructs include the erythropoietin promoter and N-WASP promoter (see, Tsuchiya, 1993, J. Biochem. 113:395 for disclosure of the erythropoietin promoter and Salvi, 2017, Biochemistry and Biophysics Reports 9:13-21 for disclosure of N-WASP promoter, both of which are incorporated by reference for the teachings of hypoxia-induced promoters). Alternatively, the recombinant vectors may contain drug inducible promoters, for example promoters inducible by administration of rapamycin and related analogs (see, for example, International Patent Application Publication Nos. WO94/18317, WO 96/20951, WO 96/41865, WO 99/10508, WO 99/10510, WO 99/36553, and WO 99/41258, and U.S. Pat. No. 7,067,526 (disclosing rapamycin analogs), which are incorporated by reference herein for their disclosure of drug inducible promoters). The inducible promoter may also be selected from known promoters including the ecdysone promoter, the estrogen-responsive promoter, and the tetracycline-responsive promoter, or heterodimeric repressor switch. See, Sochor et al, An Autogenously Regulated Expression System for Gene Therapeutic Ocular Applications. Scientific Reports, 2015 Nov. 24; 5:17105 and Daber R, Lewis M., A novel molecular switch. J Mol Biol. 2009 Aug. 28; 391 (4): 661-70, Epub 2009 Jun. 21 which are both incorporated herein by reference in their entirety.

In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2% binding site. In certain embodiments, the HIF binding site comprises an RCGTG motif. For details regarding the location and sequence of HIF binding sites, see, e.g., Schödel, et al., Blood, 2011, 117 (23): e207-e217, which is incorporated by reference herein in its entirety. In certain embodiments, the promoter comprises a binding site for a hypoxia induced transcription factor other than a HIF transcription factor. In certain embodiments, the recombinant vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety.

In another embodiment, the promoter is cell-specific. The term “cell-specific” means that the particular promoter selected for the recombinant vector can direct expression of the optimized transgene coding sequence in a particular cell or tissue type. In some embodiments, the promoter is a ubiquitous or constitutive promoter.

In certain embodiments, the promoter is a CB7 promoter (see Dinculescu et al., 2005, Hum Gene Ther 16:649-663, incorporated by reference herein in its entirety). In certain embodiments, the CB7 promoter includes other expression control elements that enhance expression of the therapeutic product driven by the vector, e.g. (1) a CAG promoter; (2) a CBA promoter; (3) a CMV promoter; (4) a 1.7-kb red cone opsin promoter (PR1.7 promoter); (5) a Rhodopsin Kinase (GRK1) photoreceptor-specific enhancer-promoter (Young et al., 2003, Retinal Cell Biology; 44:4076-4085); (6) an hCARp promoter, which is a human cone arrestin promoter; (7) an hRKp, which is a rhodopsin kinase promoter; (8) a cone photoreceptor specific human arrestin 3 (ARR3) promoter; (9) a rhodopsin promoter; and (10) a U6 promoter (in particular when the therapeutic product is a small RNA such as shRNA or siRNA).

In certain embodiments, the promoter is a hybrid chicken β-actin (CBA) promoter with cytomegalovirus (CMV) enhancer elements. In another embodiment, the promoter is the CB7 promoter. Other suitable promoters include the human β-actin promoter, the human elongation factor-la promoter, the cytomegalovirus (CMV) promoter, the simian virus 40 promoter, and the herpes simplex virus thymidine kinase promoter. See, e.g., Damdindorj et al, (August 2014) A Comparative Analysis of Constitutive Promoters Located in Adeno-Associated Viral Vectors. PLOS ONE 9 (8): e106472. Still other suitable promoters include viral promoters, constitutive promoters, regulatable promoters (see, e.g., WO 2011/126808 and WO 2013/04943). Alternatively a promoter responsive to physiologic cues may be utilized in the expression cassette, rAAV genomes, vectors, plasmids and viruses described herein. In one embodiment, the promoter is of a small size, under 1000 bp, due to the size limitations of the AAV vector. In another embodiment, the promoter is under 400 bp. Other promoters may be selected by one of skill in the art.

In a further embodiment, the promoter is selected from SV40 promoter, the dihydrofolate reductase promoter, a phage lambda (PL) promoter, a herpes simplex viral (HSV) promoter, a tetracycline-controlled trans-activator-responsive promoter (tet) system, a long terminal repeat (LTR) promoter, such as a RSV LTR, MoMLV LTR, BIV LTR or an HIV LTR, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter. The promoter sequences thereof are known to one of skill in the art or available publically, such as in the literature or in databases, e.g., GenBank, PubMed, or the like.

In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the recombinant vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the recombinant vectors provided herein comprise one or more long terminal repeat (LTR) promoters selected from the group consisting of AAV, MLV, MMTV, SV40, RSV, HIV-1, and HIV-2 LTRs. In certain embodiments, the recombinant vectors provided herein comprise one or more tissue specific promoters (e.g., a retinal pigment epithelial cell-specific promoter). In certain embodiments, the recombinant vectors provided herein comprise a RPE65 promoter. In certain embodiments, the recombinant vectors provided herein comprise a VMD2 promoter.

In certain embodiments, the recombinant vectors provided herein comprise one or more regulatory elements other than a promoter. In certain embodiments, the recombinant vectors provided herein comprise an enhancer. In certain embodiments, the recombinant vectors provided herein comprise a repressor. In certain embodiments, the recombinant vectors provided herein comprise an intron or a chimeric intron. In certain embodiments, the recombinant vectors provided herein comprise a polyadenylation sequence.

### B. Untranslated Regions

In certain embodiments wherein the therapeutic product is a therapeutic protein, the recombinant vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the half-life of the mRNA encoding the therapeutic protein. In certain embodiments, the UTRs are optimized for the stability of the mRNA encoding the therapeutic protein. In certain embodiments, the UTRs are optimized for the secondary structure of the mRNA encoding the therapeutic protein

### C. Inverted Terminal Repeats

In certain embodiments, the recombinant viral vectors provided herein comprise one or more inverted terminal repeat (ITR) sequences. ITR sequences may be used for packaging the recombinant therapeutic product expression cassette into the virion of the recombinant viral vector. In certain embodiments, the ITR is from an AAV, e.g., AAV9, AAV8 or AAV2 (see, e.g., Yan et al., 2005, J. Virol., 79 (1): 364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety.

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

### 5.1.4 Transgene and Therapeutic Product

In some embodiments, provided herein is a method of delivering a therapeutic product or transgene to a subject (e.g., eye of a subject). In some embodiments, the rAAV of the disclosure mediates delivery of a transgene to ocular tissue or to the eye of a subject (e.g., to the retina). In some embodiments, provided herein is a method for delivering a transgene to ocular tissue (e.g., corneal or retinal tissue), e.g., for treating or preventing a disease or disorder associated with the eye. In some embodiments, methods provided herein include administering to the eye of a subject an effective amount of rAAV, wherein the rAAV comprises (i) a variant capsid protein (e.g., a variant AAV9 capsid protein) and (ii) a nucleic acid comprising a promoter operably linked to a transgene or a nucleic acid encoding a transgene or therapeutic product. In some embodiments, the rAAV of the disclosure further comprises two AAV inverted terminal repeats (ITRs), wherein the ITRs flank the transgene. In some embodiments, the transgene encodes a gene or at least part of a gene associated with an ocular disease or a disease or disorder associated with the eye. In some embodiments, an rAAV of the disclosure comprises a nucleic acid encoding a transgene (e.g., miR-21, pri miR-184, pri miR-204) associated with ocular disease. A transgene incorporated into an rAAV of the disclosure is not limited and may be any heterologous nucleotide sequence of interest (e.g., a heterologous gene of interest). The transgene is a nucleic acid sequence, heterologous to the vector genome sequences flanking the transgene, which encodes a polypeptide, protein, or other product, of interest. The nucleic acid coding sequence is operatively linked to one or more regulatory components (e.g., promoter, enhancer, poly-A, 3′UTR, Woodchuck Hepatitis Virus Posttranscriptional Regulatory Element (WPRE)) in a manner which permits transgene transcription, translation, and/or expression in a host cell. The composition of the heterologous transgene sequence will depend upon the application (e.g., the therapeutic application or indication to be treated). In some embodiments, an rAAV of the disclosure comprises two or more heterologous transgenes, for example, two, three, four or five heterologous transgenes. In other embodiments, an rAAV of the disclosure comprises one heterologous transgene incorporated into the rAAV viral particle.

The size of the nucleotide sequence of a transgene can vary. For example, the nucleotide sequence of a transgene encoding a therapeutic protein can be at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide sequence of a transgene encoding a therapeutic protein is at least about 1.4 kb in length. In certain embodiments, the nucleotide sequence of a transgene encoding a therapeutic protein is about 1.4 kb to 5 kb in length. In some embodiments, the nucleotide sequences of a transgene encoding a therapeutic protein is 1.4 kb to 5 kb or 5 kb to 10 kb. Alternatively, the nucleotide sequence of a transgene is at least about 30 nucleotides, at least about 40 nucleotides, at least about 50 nucleotides, at least about 75 nucleotides in length, at least about 100 nucleotides in length, at least about 150 nucleotides in length, at least about 200 nucleotides in length, at least about 250 nucleotides in length, at least about 300 nucleotides in length, at least about 350 nucleotides in length, at least about 400 nucleotides in length, at least about 500 nucleotides in length, at least about 600 nucleotides in length, at least about 700 nucleotides in length, at least about 800 nucleotides in length, at least about 900 nucleotides in length, at least about 1000 nucleotides in length, or at least about 1200 nucleotides in length. In some embodiments, the nucleotide sequence of a transgene is about 30 to 150 nucleotides in length or about 150 to 500 nucleotides in length. In certain embodiments, the nucleotide sequence of a transgene is about 100 to 500 nucleotides in length or 500 to 1000 nucleotides in length. In some embodiments, the nucleotide sequence of a transgene is 500 nucleotides to 1200 nucleotides in length.

In some embodiments, an rAAV of the disclosure comprises a therapeutic transgene. A therapeutic transgene of the disclosure can be a sequence that encodes a biomolecule (e.g., a therapeutic biomolecule) which is useful in biology and treatment of a disease, such as a protein (e.g., an enzyme), polypeptide, peptide, RNA (e.g., tRNA, dsRNA, ribosomal RNA, catalytic RNAs, siRNA, miRNA, pre-miRNA, lncRNA, snoRNA, small hairpin RNA, trans-splicing RNA, and antisense RNA), one or more components of a gene or base editing system, e.g., CRISPR gene editing system, antisense oligonucleotides (AONs), antisense oligonucleotide (AON)-mediated exon skipping, a poison exon(s) that triggers nonsense mediated decay (NMD), or a dominant negative mutant. In some embodiments, a transgene comprises a nucleic acid sequence encoding a sequence useful for gene therapy applications. For example, certain diseases come about when one or more loss-of-function mutations within a gene reduce or abolish the amount or activity of the protein encoded by the gene. In certain embodiments, a transgene utilized herein encodes a functional version of the protein. In other embodiments, an rAAV of the disclosure comprises a transgene comprising a nucleic acid sequence encoding a sequence useful for gene therapy applications that benefit from gene silencing. For example, certain diseases come about when gain-of-function mutations within a gene result in an aberrant amount or activity of the protein encoded by the gene. In certain embodiments, a transgene utilized herein encodes an inhibitory polynucleotide, e.g., an inhibitory RNA such as an miRNA or siRNA, or one or more components of gene editing system, e.g., a CRISPR gene editing system. In some embodiments, a transgene comprises a nucleic acid encoding a CRISPR-Cas system for targeted gene disruption or correction. In other embodiments, a transgene comprising a nucleic acid sequence encodes a sequence useful for gene therapy applications that benefit from gene addition. In certain embodiments, a transgene utilized herein encodes a gene product, e.g., a protein, not present in a recipient, e.g., a human subject, of the gene therapy. In some embodiments, a transgene comprises a nucleic acid sequence encoding an RNA sequence useful in biology and medicine, such as, e.g., tRNA, dsRNA, ribosomal RNA, catalytic RNA, siRNA, miRNA, pre-miRNA, lncRNA, snoRNA, small hairpin RNA, trans-splicing RNA, and antisense RNA. One example of a useful RNA sequence is a sequence which inhibits or extinguishes expression of a targeted nucleic acid sequence in a treated subject. Suitable target nucleic acid sequences may include oncologic sequences and viral sequences. In some embodiments, a transgene comprises a nucleic acid sequence encoding a small nuclear RNA (snRNA) construct which induces exon skipping. In certain embodiments, an RNAi agent targets a gene of interest at a location of a single-nucleotide polymorphism (SNP) or a variant within the nucleotide sequence.

In some embodiments, an RNAi agent is an siRNA duplex, wherein the siRNA duplex contains an antisense strand (guide strand) and a sense strand (passenger strand) hybridized together forming a duplex structure, wherein the antisense strand is at least partially complementary to the nucleic acid sequence of the targeted gene, and wherein the sense strand is at least partially homologous to the nucleic acid sequence of the targeted gene. In some embodiments, the 5′ end of the antisense strand has a 5′phosphate group and the 3′end of the sense strand contains a 3′ hydroxyl group. In some embodiments, there are none, one or 2 nucleotide overhangs at the 3′ end of one or both strands. In some embodiments, one or more than one nucleotide of an antisense strand and/or a sense strand is modified. Non-limiting examples of nucleotide modifications include 2′deoxy, 2′-fluoro, 2′ O-methyl, 2′deoxy-2′fluoro, a phosphorothioate, 5′-morpholinno, a universal base modified nucleotide, a terminal cap molecule at the 3′-end, the 5′-end, or both 3′ and 5′-ends, an inverted abasic, or an inverted abasic locked nucleic acid modification at the 5′-end and/or 3′ end.

In some embodiments, each strand of an siRNA duplex targeting a gene of interest is about 19 to 25, 19 to 24 or 19 to 21 nucleotides in length. In some embodiments, an siRNA or dsRNA includes at least two sequences that are complementary to each other. The dsRNA includes a sense strand having a first sequence and an antisense strand having a second sequence. In some embodiments, the antisense strand includes a nucleotide sequence that is substantially complementary to at least part of an mRNA encoding the target gene, and the region of complementarity is 30 nucleotides or less, and at least 15 nucleotides in length. In some embodiments, the dsRNA is 19 to 25, 19 to 24 or 19 to 21 nucleotides in length. In some embodiments, the dsRNA is from about 15 to about 25 nucleotides in length. In some embodiments, the dsRNA is from about 25 to about 30 nucleotides in length. In some embodiments, the dsRNA is about, at least about, or at most about 15 nucleotides in length, 16 nucleotides in length, 17 nucleotides in length, 18 nucleotides in length, 19 nucleotides, 20 nucleotides, 21 nucleotides, 22 nucleotides, 23 nucleotides, 24 nucleotides, 25 nucleotides in length, 26 nucleotides in length, 27 nucleotides in length, 28 nucleotides in length, 29 nucleotides in length, or 30 nucleotides in length.

In some embodiments, an rAAV of the disclosure comprises a transgene comprising a nucleic acid sequence encoding a protein, peptide or other product that corrects or ameliorates a genetic deficiency or other abnormality in a subject. Such genetic deficiencies may include deficiencies in which gene products are expressed at less than levels considered normal for a particular subject (e.g., a human subject) or deficiencies in which a functional gene product is not expressed. In some embodiments, an rAAV of the disclosure comprises multiple transgenes to, e.g., correct or ameliorate a genetic defect caused by a multi-subunit protein. In some instances, a different transgene may be used to encode each subunit of a protein, or to encode different peptides or proteins. This may be desirable when the size of the nucleic acid sequence encoding the protein subunit is large, non-limiting examples include e.g., for an immunoglobulin, the platelet-derived growth factor, or a dystrophin protein. A host cell may be infected with an rAAV of the disclosure containing transgenes, wherein each transgene comprises a nucleic acid sequence encoding a different subunit of a multi-subunit protein, in order to produce the multi-subunit protein. Alternatively, an rAAV of the disclosure may comprise a single transgene comprising nucleic acid sequences encoding different subunits of a multi-subunit protein. In this case, a single transgene comprises nucleic acid sequences encoding each of the subunits and the nucleic acid sequence encoding each subunit may be separated by an internal ribozyme entry site (IRES). This may be desirable when the size of the nucleic acid sequence encoding each of the subunits is small, e.g., the total size of the nucleic acid sequences encoding the subunits and the IRES is less than five kilobases. As an alternative to an IRES, the nucleic acid sequence may be separated by sequences encoding a peptide, such as, e.g., 2A peptide, which self-cleaves in a post-translational event. See, e.g., Donnelly et al, J. Gen. Virol., 78 (Pt 1): 13-21 (January 1997); Furler, et al, Gene Ther., 8 (11): 864-873 (June 2001); Klump et al., Gene Ther., 8 (10): 811-817 (May 2001). A 2A peptide is significantly smaller than an IRES, making it well suited for use when space is a limiting factor. More often, when a transgene is large, consists of multi-subunits, or both, two or more AAV viral particles (including an rAAV of the disclosure) each carrying a desired transgene may be co-administered to allow them to concatamerize in vitro or in vivo to form a single vector genome. See, e.g., Yang et al., J Virol. 1999 November; 73 (11): 9468-9477 for information regarding the concatamerization of AAV. For example, a first AAV viral particle may comprise a single transgene and a second AAV viral particle may comprise a different transgene for co-expression in a host cell.

In some embodiments, a transgene comprises a nucleic acid sequence encoding a protein heterologous to AAV (e.g., a therapeutic protein). In certain embodiments, a transgene comprises a nucleic acid sequence encoding a therapeutic protein that is endogenously expressed in, for example, an eye/ocular cell/tissue of a subject.

In some embodiments, a transgene comprises a nucleic acid sequence, which upon expression produces a detectable signal. In some instances, such a nucleic acid sequence encodes an enzyme (such as, e.g., β-lactamase, β-galactosidase (LacZ), alkaline phosphatase, thymidine kinase, chloramphenicol acetyltransferase (CAT), and luciferase), a fluorescent protein (such as, e.g., green fluorescent protein (GFP), yellow fluorescent protein, and red fluorescent protein), a membrane bound protein (such as, e.g., CD2, CD4, CD8, the influenza hemagglutinin protein, and others well known in the art, to which high affinity antibodies directed thereto exist or can be produced by conventional means) or a fusion protein comprising a membrane bound protein appropriately fused to an antigen tag domain. These nucleic acid sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry (IHC). For example, where the nucleic acid sequence encodes LacZ, the presence of an AAV vector genome expressing LacZ may be detected by assays for beta-galactosidase activity. In another example, where the transgene comprises a nucleic acid sequence encoding green fluorescent protein or luciferase, an AAV vector genome expressing the green fluorescent protein or luciferase may be detected visually by color or light production in a luminometer. An AAV viral particle comprising a transgene that comprises a nucleotide sequence encoding a product with a detectable signal may be used a selectable marker or may be used to trace the virus.

A gene associated with ocular disease or a disease or disorder associated with the eye can be a protein, polypeptide, antibody or fragment thereof (e.g., ScFv), toxin, or interfering RNA (e.g., siRNA, dsRNA, miRNA, artificial miRNA (ami-RNA), antagomir). Examples of genes associated with ocular disease or a disease or disorder associated with the eye include, but are not limited to Frizzled 4 (Fzd4), angiopoietin-1 (Angpt1, isoform 1 and/or isoform 2), associated with corneal trauma; transforming growth factor β (TGF-β), Smad and mitogen-activated protein kinases (e.g., MAPK), associated with fibrotic disorders of the eye; IL-1α, IL-1β, IL-6, TNFα, interferon γ, transforming growth factor β1, and CD4, associated with traumatic corneal injury (e.g., alkali burn), protein p27, Cytokeratin 13, interleukin-like growth factor 2 (ILGF-2), junB, Metallothionein hMT-Ie, keratin 6 (e.g., KRT6), and beta 2-microglobulin, associated with corneal disease; and, connective tissue growth factor (CTGF) and vascular endothelial growth factor (VEGF). In some embodiments, a transgene encodes a pri-miRNA. In some embodiments, the pri-miRNA is pri-miRNA-184 or pri-miRNA-204. In some embodiments, a rAAV of the disclosure comprises a transgene that encodes a TuD miRNA (e.g., miR-21 TuD miRNA). In some embodiments, the gene associated with ocular disease or a disease or disorder associated with the eye is selected from the group consisting of miR-106b, miR-1955, let-7i, miR-126-3p, miR-152, miR-24, miR99b, miR223, miR126-5p, miR146a, miR-150, miR191, miR-140, miR-221, miR301a, miR-484, miR-327, miR-2132, miR-28, miR-27b, miR-423-5p, miR-132, miR-19a, miR-1-3, miR-1-6, miR-17, miR-19b, miR-214, miR-21, miR-350, miR-425, miR-335-5p, miR-382, miR-2146, miR-804, miR-378, miR-184, miR-203, and miR-204.

In some embodiments, genes associated with a disease or disorder associated with the eye includes at least one of RPE65, NR2E3, GUCY2D CACNA1F, and/or ABCA4; at least one of USH2A, CEP290, and/or ABCA4; at least one of RPE65, PRPH2, PRPF31, BEST1, and/or ABCA4; at least one of RPGR, RPE65, RHO, NR2E3 GUCA1A, and/or CRX; at least one of USH2A, RPGR, RPE65, RLBP1, RHO, and/or PRPH2; at least one of GUCY2D, CRB1, AIPL1, and/or ABCA4; at least one of RPE65, PRPH2, NR2E3 GUCY2D, GUCA1A, and/or CRX; at least one of USH2A, RPGR, RPE65, RLBP1, RHO, and/or PRPH2; at least one of ABCA4, AIPL1, BEST1, CACNA1F, CEP290, and/or CFH; or at least one mutation associated with at least one of RHO, NR2E3, and/or GUCA1A. In some embodiments, genes associated with a disease or disorder associated with the eye includes at least one of: GUCY2D (Guanylate Cyclase 2D, Retinal), PRPH2 (Peripherin 2), GUCA1A (Guanylate Cyclase Activator 1A), CACNA1F (Calcium Voltage-Gated Channel Subunit Alpha1 F), RHO (Rhodopsin), RPE65 (Retinoid Isomerohydrolase RPE65), ABCA4 (ATP Binding Cassette Subfamily A Member 4), CFH (Complement Factor H), RPGR (Retinitis Pigmentosa GTPase Regulator), RLBP1, and/or PDE6B (Phosphodiesterase 6B).

Genes associated with a retinal disorders include, but are not limited to, DHX38, DRAM2, KIZ, RDH11, TTLL5, TEK tyrosine kinase, endothelial (TEK); complement factor B (CFB); hypoxia-inducible factor 1, a subunit (HIF 1A); HtrA serine peptidase 1 (HTRA1); platelet-derived growth factor receptor β (PDGFRB); chemokine, CXC motif, receptor 4 (CXCR4); insulin-like growth factor I receptor (IGF1R); angiopoietin 2 (ANGPT2); v-fos FBJ murine osteosarcoma viral oncogene homolog (FOS); cathepsin L1, transcript variant 1 (CTSL1); cathepsin L1, transcript variant 2 (CTSL2); intracellular adhesion molecule 1 (ICAM1); insulin-like growth factor I (IGF1); integrin α5 (ITGA5); integrin 131 (ITGB1); nuclear factor kappa-B, subunit 1 (NFKB1); nuclear factor kappa-B, subunit 2 (NFKB2); chemokine, CXC motif, ligand 12 (CXCL12); tumor necrosis factor receptor 1 (TNFR1); vascular endothelial growth factor (VEGF); vascular endothelial growth factor receptor 1 (VEGFR1); tumor necrosis factor-alpha-converting enzyme (TACE); and kinase insert domain receptor (KDR). Target genes associated with glaucoma include, but it is not limited to, carbonic anhydrase II (CA2); carbonic anhydrase IV (CA4); carbonic anhydrase XII (CA12); β1 andrenergic receptor (ADBR1); β2 andrenergic receptor (ADBR2); acetylcholinesterase (ACHE); Na+/K+-ATPase; solute carrier family 12 (sodium/potassium/chloride transporters), member 1 (SLC12A1); solute carrier family 12 (sodium/potassium/chloride transporters), member 2 (SLC12A2); connective tissue growth factor (CTGF); serum amyloid A (SAA); secreted frizzled-related protein 1 (sFRP1); gremlin (GREM1); lysyl oxidase (LOX); c-Maf; rho-associated coiled-coil-containing protein kinase 1 (ROCK1); rho-associated coiled-coil-containing protein kinase 2 (ROCK2); plasminogen activator inhibitor 1 (PAI-1); endothelial differentiation, sphingolipid G-protein-coupled receptor, 3 (Edg3 R); myocilin (MYOC); NADPH oxidase 4 (NOX4); Protein Kinase Cδ (PKCδ); Aquaporin 1 (AQP1); Aquaporin 4 (AQP4); members of the complement cascade; ATPase, H+ transporting, lysosomal V1 subunit A (ATP6V1A); gap junction protein α-1 (GJA1); formyl peptide receptor 1 (FPR1); formyl peptide receptor-like 1 (FPRL1); interleukin 8 (IL8); nuclear factor kappa-B, subunit 1 (NFKB1); nuclear factor kappa-B, subunit 2 (NFKB2); presenilin 1 (PSEN1); tumor necrosis factor-alpha-converting enzyme (TACE); transforming growth factor β2 (TGFB2); transient receptor potential cation channel, subfamily V, member 1 (TRPV1); chloride channel 3 (CLCN3); gap junction protein α5 (GJA5); tumor necrosis factor receptor 1 (TNFR1); and chitinase 3-like 2 (CHI3L2). Target genes associated with ocular inflammation include, but it is not limited to, tumor necrosis factor receptor superfamily, member 1A (TNFRSF1A); phosphodiesterase 4D, cAMP-specific (PDE4D); histamine receptor H1 (HRH1); spleen tyrosine kinase (SYK); interkeukin 1β (IL1B); nuclear factor kappa-B, subunit 1 (NFKB1); nuclear factor kappa-B, subunit 2 (NFKB2); and tumor necrosis factor-alpha-converting enzyme (TACE).

In some embodiments, an rAAV of the disclosure comprises a nucleic acid encoding a transgene that has a region of complementarity to a gene associated with ocular disease or a disease or disorder associated with the eye (e.g., Fzd4 or Angpt1). A “region of complementarity” refers to a region on a nucleic acid antisense strand (e.g., miRNA) that is substantially complementary (e.g., 60%, 70%, 80%, 90%, 95%, 99%, or 100% complementary) to a sequence, for example a target sequence (e.g., Fzd4, Angpt1). A region of complementarity can be 1, 2, 3, 4, 5, 6, 7, 8, 9, 10, 15, 16, 17, 18, 19, 20, 21, 22, 23, 24, 25, 26, 27, 28, 29, 30, 40, or 50 nucleotides in length. In some embodiments, a region of complementarity is greater than 50 nucleotides in length.

### 5.1.5 Pharmaceutical Compositions and Kits

In certain embodiments, provided herein are pharmaceutical compositions comprising an rAAV of the disclosure and a pharmaceutically acceptable carrier. The pharmaceutical composition may be prepared as individual, single unit dosage forms. The pharmaceutical composition provided herein can be formulated for, for example, parenteral, subcutaneous, intramuscular, intravenous, intraperitoneal, intranasal, intrathecal, transdermal, suprachoroidal, retinal, subretinal, juxtascleral, intravitreal, subconjunctival, and/or intraretinal administration. In some embodiments, pharmaceutical composition provided herein can be formulated for intravenously, intrathecally, intracerebroventicularly, or intraparenchymally administration.

Compositions are described comprising a recombinant vector encoding a therapeutic product described herein and a suitable carrier. A suitable carrier (e.g., for suprachoroidal, subretinal, juxtascleral, intravitreal, subconjunctival, and/or intraretinal administration) would be readily selected by one of skill in the art.

The disclosure provides a pharmaceutical composition comprising a recombinant adeno-associated virus (AAV), potassium phosphate monobasic, sodium chloride, sodium phosphate dibasic anhydrous, sucrose, and surfactant.

In some embodiments, the disclosure provides a pharmaceutical composition comprising a recombinant adeno-associated virus (AAV), ionic salt excipient or buffering agent, sucrose, and poloxamer 188. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate.

In certain embodiments, the pharmaceutical composition has a ionic strength about 60 mM to 115 mM. In certain embodiments, the pharmaceutical composition has a ionic strength about 60 mM to 100 mM. In certain embodiments, the pharmaceutical composition has a ionic strength about 65 mM to 95 mM. In certain embodiments, the pharmaceutical composition has a ionic strength about 70 mM to 90 mM. In certain embodiments, the pharmaceutical composition has a ionic strength about 75 mM to 85 mM.

In certain embodiments, the pharmaceutical composition has a ionic strength about 30 mM to 100 mM. In certain embodiments, the pharmaceutical composition has a ionic strength about 35 mM to 95 mM. In certain embodiments, the pharmaceutical composition has a ionic strength about 40 mM to 90 mM. In certain embodiments, the pharmaceutical composition has a ionic strength about 45 mM to 85 mM. In certain embodiments, the pharmaceutical composition has a ionic strength about 50 mM to 80 mM. In certain embodiments, the pharmaceutical composition has a ionic strength about 55 mM to 75 mM. In certain embodiments, the pharmaceutical composition has a ionic strength about 60 mM to 70 mM.

In certain embodiments, the pharmaceutical composition has a ionic strength ranging from 60 mM to 115 mM. In certain embodiments, the pharmaceutical composition has a ionic strength ranging from 60 mM to 100 mM. In certain embodiments, the pharmaceutical composition has a ionic strength ranging from 65 mM to 95 mM. In certain embodiments, the pharmaceutical composition has a ionic strength ranging from 70 mM to 90 mM. In certain embodiments, the pharmaceutical composition has a ionic strength ranging from 75 mM to 85 mM.

In certain embodiments, the pharmaceutical composition has a ionic strength range from 30 mM to 100 mM. In certain embodiments, the pharmaceutical composition has a ionic strength range from 35 mM to 95 mM. In certain embodiments, the pharmaceutical composition has a ionic strength range from 40 mM to 90 mM. In certain embodiments, the pharmaceutical composition has a ionic strength range from 45 mM to 85 mM. In certain embodiments, the pharmaceutical composition has a ionic strength range from 50 mM to 80 mM. In certain embodiments, the pharmaceutical composition has a ionic strength range from 55 mM to 75 mM. In certain embodiments, the pharmaceutical composition has a ionic strength range from 60 mM to 70 mM.

In certain embodiments, the pharmaceutical composition comprises potassium chloride at a concentration of 0.2 g/L.

In certain embodiments, the pharmaceutical composition comprises potassium phosphate monobasic at a concentration of 0.2 g/L.

In certain embodiments, the pharmaceutical composition comprises sodium chloride at a concentration of 5.84 g/L, and

In certain embodiments, the pharmaceutical composition comprises sodium phosphate dibasic anhydrous at a concentration of 1.15 g/L.

In certain embodiments, the pharmaceutical composition comprises sucrose at a concentration of 3% (weight/volume, 30 g/L) to 18% (weight/volume, 180 g/L). In certain embodiments, the pharmaceutical composition comprises sucrose at a concentration of 4% (weight/volume, 40 g/L).

In certain embodiments, the pharmaceutical composition comprises poloxamer 188 at a concentration of 0.001% (weight/volume, 0.01 g/L).

In certain embodiments, the pharmaceutical composition comprises poloxamer 188 at a concentration of 0.0005% (weight/volume, 0.005 g/L) to 0.05% (weight/volume, 0.5 g/L). In certain embodiments, the pharmaceutical composition comprises poloxamer 188 at a concentration of 0.001% (weight/volume, 0.01 g/L).

In some embodiments, the disclosure provides a pharmaceutical composition comprises a recombinant adeno-associated virus (AAV), ionic salt excipient or buffering agent, sucrose, and surfactant. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate. In some embodiments, the surfactant can be one or more components from the group consisting of poloxamer 188, polysorbate 20, and polysorbate 80.

In certain embodiments, the pharmaceutical composition comprises polysorbate 20 at a concentration of 0.0005% (weight/volume, 0.05 g/L) to 0.05% (weight/volume, 0.5 g/L).

In certain embodiments, the pharmaceutical composition comprises polysorbate 80 at a concentration of 0.0005% (weight/volume, 0.05 g/L) to 0.05% (weight/volume, 0.5 g/L).

In certain embodiments, the pH of the pharmaceutical composition is about 7.4.

In certain embodiments, the pH of the pharmaceutical composition is about 6.0 to 9.0.

In certain embodiments, the pH of the pharmaceutical composition is 7.4.

In certain embodiments, the pH of the pharmaceutical composition is 6.0 to 9.0.

As used herein and unless otherwise specified, the term “about” means within plus or minus 10% of a given value or range.

In certain embodiments, the pharmaceutical composition is in a hydrophobically-coated glass vial.

In certain embodiments, the pharmaceutical composition is in a Cyclo Olefin Polymer (COP) vial.

In certain embodiments, the pharmaceutical composition is in a Daikyo Crystal Zenith® (CZ) vial.

In certain embodiments, the pharmaceutical composition is in a TopLyo coated vial.

In certain embodiments, disclosed herein is a pharmaceutical composition consists of: (a) the recombinant AAV, (b) potassium chloride at a concentration of 0.2 g/L, (c) potassium phosphate monobasic at a concentration of 0.2 g/L, (d) sodium chloride at a concentration of

5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the recombinant AAV is AAV9.

In certain embodiments, the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 1×10¹¹ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 3×10¹² GC/mL, about 2×10¹³ GC/mL or about 3×10¹³ GC/mL. In some embodiments, 2×10⁹ and 1×10¹⁰ of vector genome is administered to a subject (e.g., intravitreally).

In certain embodiments, the disclosure provides a pharmaceutical composition or formulation comprising a recombinant adeno-associated virus (AAV) of the disclosure, potassium phosphate monobasic, sodium chloride, sodium phosphate dibasic anhydrous, sucrose, and poloxamer 188. In some embodiments, the recombinant AAV comprises components from AAV9. In some embodiments, the AAV used for delivering the transgene has a tropism for retinal cells, photoreceptor cells, or a cell present in the eye of a subject. Such AAV can include non-replicating recombinant adeno-associated virus vectors, particularly those bearing an AAV9 capsid or a variant thereof.

In some embodiments, the pharmaceutical composition consists of: (a) an AAV capsid packaging vector encoding a transgene of interest, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water.

In some embodiments, the pharmaceutical composition is a liquid composition. In some embodiments, the pharmaceutical composition is a frozen composition. In some embodiments, the pharmaceutical composition is a lyophilized composition from a liquid composition disclosed herein. In some embodiments, the pharmaceutical composition is a reconstituted lyophilized formulation.

In some embodiments, the pharmaceutical composition is a lyophilized composition comprising a residual moisture content between about 1% and about 7%. In some embodiments, the pharmaceutical composition is a lyophilized composition comprising a residual moisture content between about 2% and about 6%. In some embodiments, the pharmaceutical composition is a lyophilized composition comprising a residual moisture content between about 3% and about 4%. In some embodiments, the pharmaceutical composition is a lyophilized composition comprising a residual moisture content about 5%.

In certain aspects, disclosed herein is a method of treating or preventing a disease in a subject, comprising administering to the subject the pharmaceutical composition. In some embodiments, a pharmaceutical composition provided herein is suitable for administration by one, two or more routes of administration (e.g., suitable for suprachoroidal and subretinal administration).

In certain aspects, disclosed herein is a method of treating or preventing a disease in a subject, comprising administering to the subject the pharmaceutical composition by intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface).

In certain embodiments, the pharmaceutical composition provided herein is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired viscosity that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired density that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired osmolality that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)). In specific embodiments, the desired osmolality for subretinal administration is 160 to 430 mOsm/kg H₂O. In other specific embodiments, the desired osmolality of suprachoroidal administration is less than 600 mOsm/kg H₂O.

In certain embodiments, the pharmaceutical composition has a osmolality of about 100 to 500 mOsm/kg H₂O. In certain embodiments, the pharmaceutical composition has a osmolality of about 130 to 470 mOsm/kg H₂O. In certain embodiments, the pharmaceutical composition has a osmolality of about 160 to 430 mOsm/kg H₂O. In certain embodiments, the pharmaceutical composition has a osmolality of about 200 to 400 mOsm/kg H₂O. In certain embodiments, the pharmaceutical composition has a osmolality of about 240 to 340 mOsm/kg H₂O. In certain embodiments, the pharmaceutical composition has a osmolality of about 280 to 300 mOsm/kg H₂O. In certain embodiments, the pharmaceutical composition has a osmolality of about 295 to 395 mOsm/kg H₂O. In certain embodiments, the pharmaceutical composition has a osmolality of less than 600 mOsm/kg H₂O. In certain embodiments, the pharmaceutical composition has a osmolality range of 200 mOsm/L to 660 mOsm/L. In certain embodiments, the pharmaceutical composition has a osmolality of about 200 mOsm/L. In certain embodiments, the pharmaceutical composition has a osmolality of about 250 mOsm/L. In certain embodiments, the pharmaceutical composition has a osmolality of about 300 mOsm/L. In certain embodiments, the pharmaceutical composition has a osmolality of about 350 mOsm/L. In certain embodiments, the pharmaceutical composition has a osmolality of about 400 mOsm/L. In certain embodiments, the pharmaceutical composition has a osmolality of about 450 mOsm/L. In certain embodiments, the pharmaceutical composition has a osmolality of about 500 mOsm/L. In certain embodiments, the pharmaceutical composition has a osmolality of about 550 mOsm/L. In certain embodiments, the pharmaceutical composition has a osmolality of about 600 mOsm/L. In certain embodiments, the pharmaceutical composition has a osmolality of about 650 mOsm/L. In certain embodiments, the pharmaceutical composition has a osmolality of about 660 mOsm/L.

In some embodiments, the recombinant vector of the disclosure is used for delivering the transgene to a cell. In some embodiments, the recombinant vector of the disclosure has tropism for human retinal cells or photoreceptor cells. Such vectors can include non-replicating recombinant adeno-associated virus vectors (“rAAV”), however, other viral vectors may be used, including but not limited to lentiviral vectors, vaccinia viral vectors, or non-viral expression vectors referred to as “naked DNA” constructs.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant vector in a formulation buffer comprising a physiologically compatible aqueous buffer, a surfactant and optional excipients. In a specific embodiment, the construct is formulated in Dulbecco's phosphate buffered saline and 0.001% poloxamer 188, pH=7.4.

The disclosure provides a pharmaceutical composition comprising a pharmaceutically acceptable carrier and an agent of the disclosure, said agent comprising a rAAV of the disclosure. In some embodiments, the pharmaceutical composition comprises rAAV combined with a pharmaceutically acceptable carrier for administration to a subject. In some embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present disclosure can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like

In certain embodiments of the disclosure, pharmaceutical compositions are provided for use in accordance with the methods of the disclosure, said pharmaceutical compositions comprising a therapeutically and/or prophylactically effective amount of an agent of the disclosure along with a pharmaceutically acceptable carrier.

In certain embodiments, the agent of the disclosure is substantially purified (i.e., substantially free from substances that limit its effect or produce undesired side-effects). In a specific embodiment, the host or subject is an animal, e.g., a mammal such as non-primate (e.g., cows, pigs, horses, cats, dogs, rats etc.) and a primate (e.g., monkey such as, a cynomolgus monkey and a human). In a certain embodiment, the host is a human.

Provided herein are kits comprising a pharmaceutical composition described herein, contained in one or more containers. The containers that the pharmaceutical composition can be packaged in can include, but are not limited to, bottles, packets, ampoules, tubes, inhalers, bags, vials, and containers. In certain embodiments, the kit comprises instructions for administering the pharmaceutical administration. In certain embodiments, the kit comprises devices that can be used to administer (e.g., to the eye) the pharmaceutical composition, including, but not limited to, syringes, catheters, needle-less injectors, drip bags, patches and inhalers. In another embodiment, a kit further comprises one or more other prophylactic or therapeutic agents useful for the treatment of a condition, in one or more containers.

The disclosure also provides agents of the disclosure packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the disclosure is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the disclosure include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The disclosure further provides a pharmaceutical pack or kit comprising one or more containers filled with one or more of the agents of the disclosure. Additionally, one or more other prophylactic or therapeutic agents useful for the treatment of the target disease or disorder can also be included in the pharmaceutical pack or kit. The disclosure also provides a pharmaceutical pack or kit comprising one or more containers filled with one or more of the ingredients of the pharmaceutical compositions of the disclosure. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

5.2 Manufacture of rAAVS

### 5.2.1 Polynucleotides, Plasmids and Cells

In some embodiments, the disclosure provides for an isolated nucleic acid comprising a nucleotide sequence encoding a variant adeno-associated virus (AAV) capsid protein of the disclosure (e.g., a variant AAV capsid comprising a heterologous amino acid sequence or a peptide insert). In some aspects, the disclosure provides for a nucleic acid for use, wherein the nucleic acid encodes a therapeutic product operatively linked to a promoter or enhancer-promoter described herein. In some embodiments, the disclosure provides for an isolated nucleic acid comprising a nucleotide sequence encoding a variant adeno-associated virus (AAV) capsid protein of the disclosure (e.g., a variant AAV capsid protein comprising an amino acid sequence that is at least 90%, 91%, 92%, 93%, 94%, 95%, 96%, 97%, 98%, 99%, or 100% identical to any one of SEQ ID NOs: 10 and 12-22). In some embodiments, the disclosure provides for an isolated nucleic acid comprising a nucleotide sequence encoding a variant adeno-associated virus (AAV) capsid protein comprising an amino acid sequence that is identical to SEQ ID NO: 10. In some embodiments, the disclosure provides for an isolated nucleic acid comprising a nucleotide sequence encoding a variant adeno-associated virus (AAV) capsid protein comprising an amino acid sequence that is identical to SEQ ID NO: 12. In some embodiments, an isolated nucleic acid comprises a nucleotide sequence of SEQ ID NO: 9 or 11. In some embodiments, an isolated nucleic acid comprises a nucleotide sequence that is at least 90%, 91%, 92%, 93%, 94%, 95%, 96%, 97%, 98%, 99%, or 100% identical to SEQ ID NO: 9 or 11. In some embodiments, the disclosure provides for a cell comprising a vector, a plasmid, and/or an isolated nucleic acid of the disclosure. In some embodiments, the disclosure provides for a plasmid comprising a nucleotide sequence that is at least 90%, 91%, 92%, 93%, 94%, 95%, 96%, 97%, 98%, 99%, or 100% identical to SEQ ID NO: 9 or 11.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence encoding the therapeutic product of interest, untranslated regions, and termination sequences. In certain embodiments, recombinant vectors provided herein comprise a promoter operably linked to the sequence encoding the therapeutic product of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161). In some embodiments, the polynucleotide is in the form of a ssDNA. In another specific embodiment, the polynucleotide is in the form of a dsDNA.

Also provided herein are plasmids comprising a polynucleotide provided herein (hereinafter “rAAV plasmids”). In a specific embodiment, the rAAV plasmid is a ssDNA plasmid. In another specific embodiment, the rAAV plasmid is a dsDNA plasmid. In some embodiments, the rAAV plasmid is in a circular form. In other embodiments, the rAAV plasmid is in a linear form.

Further provided herein are cells (preferably ex vivo cells) expressing (e.g., recombinantly) an rAAV provided herein. In certain embodiments, the cell (e.g., ex vivo cell) comprises a polynucleotide provided herein or an rAAV plasmid provided herein. In certain embodiments, the cell (e.g., ex vivo cell) further comprises helper polynucleotide(s) or helper plasmids providing the AAV Rep, Cap, and Ad5 functions. The cell (e.g., ex vivo cells) can by a mammalian host cell, for example, retinal cell, choroidal cell, photoreceptor cell, a cell associated with the eye, HEK293, HEK293-T, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The mammalian host cell can be derived from, for example, human, monkey, mouse, rat, rabbit, or hamster. In a specific embodiment, the mammalian host cell is a retinal cell. In a specific embodiment, the mammalian host cell is a human embryonic kidney 293 (HEK293) cell or HEK293-T cell.

5.2.2 Methods of Making rAAVs and Assessment of Efficacy

Provided herein are methods of making an rAAV of the disclosure. In certain embodiments, the method comprises transfecting a cell (e.g., an ex vivo cell) with an rAAV plasmid provided in Section 5.2.1 and one or more helper plasmids collectively providing the AAV Rep, Cap, and Ad5 functions. In certain embodiments, the one or more helper plasmids collectively comprises the nucleotide sequences of AAV genes Rep, Cap, VA, E2a and E4.

In some embodiments, the recombinant viral vector used in the methods described herein is or is derived from a recombinant adenovirus vector. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). In some embodiments, the transgene or therapeutic product is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12: S18-S27, which is incorporated by reference herein in its entirety.

The manufacture of an rAAV provided herein for gene therapy applications can use methods known in the art, for example, as described in Clement et al., 2016, Molecular Therapy-Methods & Clinical Development, 27:16002, which is incorporated by reference herein in its entirety. In certain embodiments, transfection of the plasmid DNA is performed using calcium phosphate plasmid precipitation on human embryonic kidney 293 cells (HEK293) or HEK293-T with the rAAV plasmid and the helper plasmid(s) that provide the AAV Rep and Cap functions as well as the Ad5 genes (VA RNAs, E2a, and E4) as is described in the art. In certain embodiments, the Rep, Cap, and Ad5 genes can be on the same helper plasmid. In certain embodiments, a two-helper method (or triple transfection) is utilized where AAV Rep, Cap, and Ad5 functions are provided from separate plasmids. In certain embodiments, the HEK293 cells can be adapted to grow in suspension in an animal component and antibiotic-free media.

In certain embodiments, rAAV can be manufactured using packaging and producer cell lines. The rAAV provided herein can be manufactured using mammalian host cells, for example, A549, WEHI, 10T1/2, BHK, MDCK, COS1, COS7, BSC 1, BSC 40, BMT 10, VERO, W138, HeLa, HEK293, HEK293-T, Saos, C2C12, L, HT1080, HepG2, primary fibroblast, hepatocyte, and myoblast cells. The rAAV provided herein may be manufactured using host cells from human, monkey, mouse, rat, rabbit, or hamster. In certain embodiments, stable cell lines can be engineered by introducing the means of producing viruses in the host cells, for example, the replication and capsid genes (e.g., the rep and cap genes of AAV) and the rAAV plasmid provided herein. In some embodiments, the rAAV can be manufactured using HEK293 cells. In some embodiments, rAAV can be produced in Sf9 insect cells by coinfecting three recombinant baculovirus plasmids with genes encoding the rep gene, the cap gene, and the rAAV genome.

The cells can be cultured, transfected, and harvested according to appropriate protocols which would be readily selected by one of skill in the art. In certain embodiments, the cells can be cultured in standard Dulbecco's modified Eagle medium (DMEM), including, but not limited to, fetal calf serum, glucose, penicillin, streptomycin, and 1-glutamine (McClure et al., J Vis Exp. 2011, (57): 3348; Shin et al., Methods Mol Biol. 2012, 798:267-284). Cells can be transfected in components which would be readily selected by one of skill in the art. In certain embodiments, transfection can take place in media solutions including, but not limited to, DMEM and Iscove's modified Dulbecco's medium (IMDM). In certain embodiments, the transfection time can take 46 hr, 47 hr, 48 hr, 49 hr, 50 hr, 51 hr, 52 hr, 53 hr, 54 hr, 55 hr, 56 hr, 57 hr, 58 hr, 59 hr, 60 hr, 61 hr, 62 hr, 63 hr, 64 hr, 65 hr, 66 hr, 67 hr, 68 hr, 69 hr, 70 hr, 50-55 hr, 55-60 hr, 60-65 hr, or 65-70 hr. After transfection, the cells can be harvested by scraping cells to remove them from the culture wells and washing the wells to collect all of the transfected cells.

For a method of producing rAAV, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation. In a specific embodiment, the rAAV described herein is an isolated or purified rAAV.

Multiple AAV serotypes have been identified. In certain embodiments, rAAVs or polynucleotides provided herein comprise one or more components derived from one or more serotypes of AAV. In certain embodiments, rAAVs or polynucleotides provided herein comprise one or more components derived from one or more of AAV1, AAV2, AAV3, AAV4, AAV5, AAV6, AAV7, AAV8, AAV9, AAVrh10, AAV11, AAV12, AAV13, AAVrh20, AAVhu.37, AAVrh39, or AAVrh74. In a certain embodiment, rAAVs or polynucleotides provided herein can comprise one or more components from one or more of AAV8, AAV9, AAV10, or AAV11 serotypes. In some embodiments, rAAVs or polynucleotides provided herein can comprise one or more components from AAV9 serotype. Nucleic acid sequences of AAV components and methods of making recombinant AAV and AAV capsids are described, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety.

In vitro assays, e.g., cell culture assays, can be used to measure therapeutic product expression from a vector described herein, thus indicating, e.g., potency of the vector. Cells utilized for the assay can include, but are not limited to, A549, WEHI, 10T1/2, BHK, MDCK, COS1, COS7, BSC 1, BSC 40, BMT 10, VERO, W138, HeLa, HEK293, HEK293-T, HuH7, Saos, C2C12, L, HT1080, HepG2, primary fibroblast, hepatocyte, and myoblast cells. In some embodiments, the cells utilized in the cell culture assay comprise HuH7 cells. In some embodiments, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess therapeutic product expression. Once expressed, characteristics of the expressed therapeutic product can be determined, including determination of the post-translational modification patterns. In addition, benefits resulting from post-translational modification of the cell-expressed therapeutic product can be determined using assays known in the art.

### 5.3 Gene Therapy

In one aspect, provided herein is a method of treating and/or preventing a disease in a subject. In some embodiments, provided herein is a method of treating and/or preventing a disease associated with the eye of a subject comprising administering to the eye (e.g., in the subretinal space) of a subject in need thereof a recombinant adeno-associated virus (rAAV) comprising a variant AAV capsid of the disclosure, wherein the variant AAV capsid comprises a peptide insert or a heterologous amino acid sequence as disclosed herein (refer to Section 5.1.1). In some embodiments, the recombinant adeno-associated virus (rAAV) comprises an AAV capsid and a vector genome packaged therein. In some embodiments, the vector genome comprises one or more of the following: (a) an AAV 5′ inverted terminal repeat (ITR) sequence; (b) a promoter; (c) a coding sequence encoding a transgene; and (d) an AAV 3′ ITR.

In some embodiments, administering an rAAV of the disclosure comprises administering to the subretinal space or retinal space of the eye of a subject. In some embodiments, the recombinant viral vector therapeutic product or transgene is administered via the suprachoroidal space in the eye of a subject. In some embodiments, the recombinant viral vector therapeutic product or transgene is administered subretinally in the eye of a subject. In some embodiments, the recombinant viral vector therapeutic product or transgene is administered via intravitreal administration to the eye of a subject. In certain embodiments, the administering step is by the use of a subretinal drug delivery device comprising a catheter that can be inserted and tunneled through the suprachoroidal space toward the posterior pole, where a small needle injects into the subretinal space. In certain embodiments, the administering step comprises inserting and tunneling the catheter of the subretinal drug delivery device through the suprachoroidal space, retinal space, subretinal space, or a suitable location in the eye. In some embodiments, the method comprises performing a vitrectomy on the eye of a subject. In certain embodiments, the vitrectomy is a partial vitrectomy. In certain embodiments, the administering step is by injecting the recombinant viral vector into the suprachoroidal space using a suprachoroidal drug delivery device. In certain embodiments, the suprachoroidal drug delivery device is a microinjector. In certain embodiments, the administering step is by the use of a juxtascleral drug delivery device that comprises a cannula whose tip can be inserted and kept in direct apposition to the scleral surface. In certain embodiments, the administering step comprises inserting and keeping the tip of the cannula in direct apposition to the scleral surface. In some embodiments, the method comprises administering the rAAV to the outer surface of the sclera of a subject in need thereof. In some embodiments, the method comprises administering the rAAV to the vitreous cavity of the eye of a subject in need thereof. In certain embodiments, the administering step is by injecting the recombinant viral vector into the vitreous cavity using an intravitreal drug delivery device. In certain embodiments, the intravitreal drug delivery device is a microinjector. In some embodiments, the method comprises administering to the subretinal space peripheral to the optic disc, fovea and macula located in the back of the eye. In some embodiments, the method does not comprise performing a vitrectomy on the eye. In certain embodiments, the injecting step is by transvitreal injection. In some embodiments, the transvitreal injection comprises inserting a sharp needle into the sclera via the superior or inferior side of the eye and passing the sharp needle all the way through the vitreous to inject the recombinant viral vector to the subretinal space on the other side. In certain embodiments, a needle is inserted at the 2 or 10 o'clock position. In certain embodiments, the transvitreal injection comprises inserting a trocar into the sclera and inserting a cannula through the trocar and through the vitreous to inject the recombinant viral vector to the subretinal space on the other side.

In certain embodiments of the methods described herein, the administering step delivers a therapeutically effective amount of the rAAV to the retina of a subject. In certain embodiments of the methods described herein, the therapeutically effective amount of the protein encoded by the rAAV genome (e.g., transgene) is produced by human retinal cells of a human subject. In certain embodiments of the methods described herein, the therapeutically effective amount of the protein encoded by the rAAV genome (e.g., transgene) is produced by human photoreceptor cells, horizontal cells, bipolar cells, amacrine cells, retina ganglion cells, and/or retinal pigment epithelial cells in the external limiting membrane of a human subject. In certain embodiments of the methods described herein, the human photoreceptor cells are cone cells and/or rod cells. In certain embodiments of the methods described herein, the retina ganglion cells are midget cells, parasol cells, bistratified cells, giant retina ganglion cells, photosensitive ganglion cells, and/or Müller glia

In some embodiments, the rAAV of the disclosure results in uniform expression of the therapeutic product throughout the eye or throughout the retina after the rAAV of the disclosure is administered to the subject. In some embodiments, the rAAV of the disclosure provides a widespread transgene expression of retinal pigmented epithelium (RPE)/choroid and/or retina.

In certain embodiments of the methods described herein, the recombinant viral vector is an rAAV vector (e.g., an rAAV8, rAAV2, rAAV9, or rAAV5 vector). In certain embodiments of the methods described herein, the recombinant viral vector is an rAAV9 vector.

In certain embodiments of the methods described herein, delivering to the eye comprises delivering to the retina, choroid, and/or vitreous humor of the eye. Methods are described for the administration of a therapeutically effective amount of a recombinant vector (i.e., a recombinant viral vector or a DNA expression construct) to human subjects having pathology of the eye. In some embodiments, the recombinant vector is a construct of the disclosure, wherein the construct comprises a variant AAV capsid (e.g., variant AAV9 capsid) comprising a heterologous amino acid sequence (e.g., the amino acid sequence of: KDEPQRRSARLSAKPAPPKPEPKPKKAPAKK (SEQ ID NO: 1); KDEPQRRSARL (SEQ ID NO: 2); SAKPAPPKPE (SEQ ID NO: 3); PKPKKAPAKK (SEQ ID NO: 4); or MKDEPQRRSARLSAKPAPPKPEPKPKKAPAKK (SEQ ID NO: 23); or about or at least about 10 contiguous amino acids of: KDEPQRRSARLSAKPAPPKPEPKPKKAPAKK (SEQ ID NO: 1); KDEPQRRSARL (SEQ ID NO: 2); SAKPAPPKPE (SEQ ID NO: 3); PKPKKAPAKK (SEQ ID NO: 4); or MKDEPQRRSARLSAKPAPPKPEPKPKKAPAKK (SEQ ID NO: 23); or an amino acid sequence comprising about, at least about, or at most about: 1, 2, 3, 4, 5, 6, 7, 8, 9, or 10 contiguous amino acids of any one of: KDEPQRRSARLSAKPAPPKPEPKPKKAPAKK (SEQ ID NO: 1); KDEPQRRSARL (SEQ ID NO: 2); SAKPAPPKPE (SEQ ID NO: 3); PKPKKAPAKK (SEQ ID NO: 4); or MKDEPQRRSARLSAKPAPPKPEPKPKKAPAKK (SEQ ID NO: 23); or an amino acid sequence comprising or consisting of about 1, 2, 3, 4, 5, 6, 7, 8, 9, or 10 contiguous amino acids of any one of: SEQ ID NOs: 1-4 and 23; or an amino acid sequence comprising at least about 1, 2, 3, 4, 5, 6, 7, 8, 9, or 10 mutations in any one of: KDEPQRRSARLSAKPAPPKPEPKPKKAPAKK (SEQ ID NO: 1); KDEPQRRSARL (SEQ ID NO: 2); SAKPAPPKPE (SEQ ID NO: 3); PKPKKAPAKK (SEQ ID NO: 4); or MKDEPQRRSARLSAKPAPPKPEPKPKKAPAKK (SEQ ID NO: 23)).

In some embodiments, the methods provided herein are for the treatment of a disease associated with the eye, without resulting in, for example, vision loss, retinal atrophy, and/or retinal degeneration.

In some embodiments, methods are described for the administration of a therapeutically effective amount of a recombinant vector (i.e., a recombinant viral vector or a DNA expression construct) to human subjects via one of the following approaches: (1) subretinal administration without vitrectomy (for example, administration to subretinal space via the suprachoroidal space or via peripheral injection), (2) suprachoroidal administration, (3) administration to the outer space of the sclera (i.e., juxtascleral administration); (4) subretinal administration accompanied by vitrectomy; (5) intravitreal administration, and (6) subconjunctival administration.

In certain embodiments, delivery to the subretinal or suprachoroidal space can be performed using the methods and/or devices described and disclosed in International Publication Nos. WO 2016/042162, WO 2017/046358, WO 2017/158365, and WO 2017/158366, each of which is incorporated by reference in its entirety.

### 5.4 Methods for Treatment

Provided herein are methods for treating a subject (e.g., human subject) with a disease, or suspected of having a disease, or experiencing at least one symptom associated with a disease. In some embodiments, the disease is a disease associated with the eye of a subject (e.g., retinal disease or ocular disease). In some embodiments, the method comprises administering to the human subject an rAAV described herein or a pharmaceutical composition described herein. Diseases/disorders associated with the eye or retina can be referred to as “ocular diseases.” Nonlimiting examples of ocular diseases include anterior ischemic optic neuropathy; acute macular neuroretinopathy; Bardet-Biedl syndrome; Behcet's disease; branch retinal vein occlusion; central retinal vein occlusion; choroideremia; choroidal neovascularization; chorioretinal degeneration; cone-rod dystrophy; color vision disorders (e.g., achromatopsia, protanopia, deuteranopia, and tritanopia); congenital stationary night blindness; diabetic uveitis; epiretinal membrane disorders; inherited macular degeneration; histoplasmosis; macular degeneration (e.g., acute macular degeneration, non-exudative age related macular degeneration, exudative age related macular degeneration); diabetic retinopathy; edema (e.g., macular edema, cystoid macular edema, diabetic macular edema); glaucoma; non-infectious uveitis; Leber congenital amaurosis; Leber's hereditary optic neuropathy; macular telangiectasia; multifocal choroiditis; non-retinopathy diabetic retinal dysfunction; ocular trauma; ocular tumors; proliferative vitreoretinopathy (PVR); retinopathy of prematurity; retinoschisis; retinitis pigmentosa; retinal arterial occlusive disease, retinal detachment, Stargardt disease (fundus flavimaculatus); sympathetic opthalmia; uveal diffusion; uveitic retinal disease; Usher syndrome; Vogt Koyanagi-Harada (VKH) syndrome; or a posterior ocular condition associated with ocular laser or photodynamic therapy. In some embodiments, provided herein is a method of treating a disease associated with the central nervous system (CNS) in a subject. In some embodiments, a method of the disclosure is administered intravenously, intrathecally, intracerebroventicularly, and/or intraparenchymally. In some embodiments, a method of the disclosure results in a focal expression of a transgene in the CNS tissue of the injection site after an rAAV vector of the disclosure is administered to a subject (e.g., via intraparenchymal injection).

In some embodiments, a subject has been diagnosed with a disease associated with the eye, is suspected of having a disease associated with the eye, and/or is experiencing at least one symptom associated with a disease associated with the eye. In some embodiments, a subject has been diagnosed with an ocular disease, is suspected of having an ocular disease, and/or is experiencing at least one symptom associated with an ocular disease. In some embodiments, the ocular disease is a retinal disease. In some embodiments, a disease associated with the eye is a retinal disease. In some embodiments, an ocular disease or disorder includes, but it is not limited to conditions associated with ocular angiogenesis, dry eye, inflammatory conditions, ocular hypertension and ocular diseases associated with elevated intraocular pressure (IOP), such as glaucoma. In some embodiments, ocular angiogenesis includes, but it is not limited to, ocular pre-angiogenic conditions and ocular angiogenic conditions, and includes ocular angiogenesis, ocular neovascularization, retinal edema, diabetic retinopathy, sequela associated with retinal ischemia, posterior segment neovascularization (PSNV), and neovascular glaucoma, for example. In some embodiments, the subject has or has been diagnosed with at least one of ocular angiogenesis, ocular neovasularization, retinal edema, diabetic retinopathy, sequela associated with retinal ischemia, posterior segment neovascularization (PSNV), and neovascular glaucoma, or subjects at risk of developing such conditions, for example. In some embodiments, ocular neovascularization includes, but it is not limited to, age-related macular degeneration, cataract, acute ischemic optic neuropathy (AION), commotio retinae, retinal detachment, retinal tears or holes, iatrogenic retinopathy and other ischemic retinopathies or optic neuropathies, myopia, retinitis pigmentosa, and/or the like. In some embodiments, a subject is diagnosed with or is suspected of having a retinal disease. In some embodiments, the ocular disease or retinal disease is selected from age-related macular degeneration, retinal vasculitis and retinal infective processes, commotio retinae, diabetic retinopathy, hereditary retinal dystrophies, ischemic insult of retinal neurons and macular edema. In some embodiments, a subject has been diagnosed with or is suspected of having a corneal disease (e.g., corneal neovascularization, corneal dystrophy, corneal inflammation, corneal abrasion, and corneal fibrosis, etc.). In some embodiments, the subject has more than one disease associated with the eye. In some embodiments, a disease associated with the eye is an ocular surface disease, ocular disease, periocular skin disease and/or eyelid disease. In some examples, the ocular disease is diabetic macular edema (DME), age-related macular degeneration (AMD), including both wet AMD and dry AMD, retinoblastoma, or diabetic retinopathy. Non-limiting examples of ocular disease include cystic macular edema, diabetic retinopathy, lattice degeneration, retinal vein occlusion, retinal artery occlusion, macular degeneration (e.g., age related macular degeneration, such as wet AMD or dry AMD), toxoplasmosis, pigmented retinitis, conjunctival laceration, corneal laceration, hyperemia, leukoplakia, corneal angiogenesis, and corneal keratoconus, uveitis including non-infectious uveitis, glaucoma and the like.

In some embodiments, the ocular disease is associated with an abnormal level of a target gene product, or by the presence of a target protein exhibiting abnormal activity. In some embodiments, a subject to be treated with the methods of the present disclosure has an abnormal level of a target gene. For example, target genes associated with retinal disorders include, but are not limited to, DHX38, DRAM2, KIZ, RDH11, TTLL5, TEK tyrosine kinase, endothelial (TEK); complement factor B (CFB); hypoxia-inducible factor 1, a subunit (HIF 1A); HtrA serine peptidase 1 (HTRA1); platelet-derived growth factor receptor β (PDGFRB); chemokine, CXC motif, receptor 4 (CXCR4); insulin-like growth factor I receptor (IGF1R); angiopoietin 2 (ANGPT2); v-fos FBJ murine osteosarcoma viral oncogene homolog (FOS); cathepsin L1, transcript variant 1 (CTSL1); cathepsin L1, transcript variant 2 (CTSL2); intracellular adhesion molecule 1 (ICAM1); insulin-like growth factor I (IGF1); integrin α5 (ITGA5); integrin 131 (ITGB1); nuclear factor kappa-B, subunit 1 (NFKB1); nuclear factor kappa-B, subunit 2 (NFKB2); chemokine, CXC motif, ligand 12 (CXCL12); tumor necrosis factor receptor 1 (TNFR1); vascular endothelial growth factor (VEGF); vascular endothelial growth factor receptor 1 (VEGFR1); tumor necrosis factor-alpha-converting enzyme (TACE); and kinase insert domain receptor (KDR). Target genes associated with glaucoma include, but it is not limited to, carbonic anhydrase II (CA2); carbonic anhydrase IV (CA4); carbonic anhydrase XII (CA12); β1 andrenergic receptor (ADBR1); β2 andrenergic receptor (ADBR2); acetylcholinesterase (ACHE); Na+/K+-ATPase; solute carrier family 12 (sodium/potassium/chloride transporters), member 1 (SLC12A1); solute carrier family 12 (sodium/potassium/chloride transporters), member 2 (SLC12A2); connective tissue growth factor (CTGF); serum amyloid A (SAA); secreted frizzled-related protein 1 (sFRP1); gremlin (GREM1); lysyl oxidase (LOX); c-Maf; rho-associated coiled-coil-containing protein kinase 1 (ROCK1); rho-associated coiled-coil-containing protein kinase 2 (ROCK2); plasminogen activator inhibitor 1 (PAI-1); endothelial differentiation, sphingolipid G-protein-coupled receptor, 3 (Edg3 R); myocilin (MYOC); NADPH oxidase 4 (NOX4); Protein Kinase Cδ (PKCδ); Aquaporin 1 (AQP1); Aquaporin 4 (AQP4); members of the complement cascade; ATPase, H+ transporting, lysosomal V1 subunit A (ATP6VIA); gap junction protein α-1 (GJA1); formyl peptide receptor 1 (FPR1); formyl peptide receptor-like 1 (FPRL1); interleukin 8 (IL8); nuclear factor kappa-B, subunit 1 (NFKB1); nuclear factor kappa-B, subunit 2 (NFKB2); presenilin 1 (PSEN1); tumor necrosis factor-alpha-converting enzyme (TACE); transforming growth factor β2 (TGFB2); transient receptor potential cation channel, subfamily V, member 1 (TRPV1); chloride channel 3 (CLCN3); gap junction protein α5 (GJA5); tumor necrosis factor receptor 1 (TNFR1); and chitinase 3-like 2 (CHI3L2). Target genes associated with ocular inflammation include, but it is not limited to, tumor necrosis factor receptor superfamily, member 1A (TNFRSF1A); phosphodiesterase 4D, cAMP-specific (PDE4D); histamine receptor H1 (HRH1); spleen tyrosine kinase (SYK); interkeukin 1β (IL1B); nuclear factor kappa-B, subunit 1 (NFKB1); nuclear factor kappa-B, subunit 2 (NFKB2); and tumor necrosis factor-alpha-converting enzyme (TACE).

In some embodiments, a gene associated with an ocular disease can be a protein, polypeptide, antibody or fragment thereof (e.g., ScFv), toxin, or interfering RNA (e.g., siRNA, dsRNA, miRNA, artificial miRNA (ami-RNA), antagomir). Examples of genes associated with ocular disease include, but are not limited to Frizzled 4 (Fzd4), angiopoietin-1 (Angpt1, isoform 1 and/or isoform 2), a gene associated with corneal trauma; transforming growth factor β (TGF-β), Smad and mitogen-activated protein kinases (e.g., MAPK), a gene associated with fibrotic disorders of the eye; IL-1α, IL-1β, IL-6, TNFα, interferon γ, transforming growth factor β1, and CD4, a gene associated with traumatic corneal injury (e.g., alkali burn), protein p27, Cytokeratin 13, interleukin-like growth factor 2 (ILGF-2), junB, Metallothionein hMT-Ie, keratin 6 (e.g., KRT6), and beta 2-microglobulin, a gene associated with corneal disease; and, connective tissue growth factor (CTGF) and vascular endothelial growth factor (VEGF).

In some embodiments, the abnormal level (e.g., of a target gene product) is a level that is either increased or decreased compared to the level in a healthy subject or a subject without a disease associated with the eye. In some embodiments, the abnormal level (e.g., of a target gene product) is a level that is either increased or decreased compared to the level in a subject or a population of subjects with a disease associated with the eye.

In some embodiments, retinitis pigmentosa or a retinal disease or a disease associated with the eye is associated with at least one of the following genes (e.g., mutation): RP1, RP2, RPGR, PRPH2, RP9, IMPDH1, PRPF31, CRB1, PRPF8, TULP1, CA4, HPRPF3, ABCA4, EYS, CERKL, FSCN2, TOPORS, SNRNP200, SEMA4A, PRCD, NR2E3, MERTK, USH2A, PROM1, KLHL7, CNGB1, BEST1, TTC8, C2Orf71, ARL6, ZFN516, DHDDS, LRAT, SPATA7, CRX, and/or PAP1.

In some embodiments, a gene associated with a retinal disease or a disease associated with the eye is associated with at least one of the following genes (e.g., mutation): GUCY2D (Guanylate Cyclase 2D, Retinal), PRPH2 (Peripherin 2), GUCA1A (Guanylate Cyclase Activator 1A), CACNA1F (Calcium Voltage-Gated Channel Subunit Alpha1 F), RHO (Rhodopsin), RPE65 (Retinoid Isomerohydrolase RPE65), ABCA4 (ATP Binding Cassette Subfamily A Member 4), CFH (Complement Factor H), RPGR (Retinitis Pigmentosa GTPase Regulator), and/or PDE6B (Phosphodiesterase 6B).

### 5.4.1 Target Patient Populations

In some embodiments, a subject is a subject with a disease or disorder associated with the eye or is at risk of having a disease or disorder associated with the eye (e.g., retinal disease). In some embodiments, a subject is a subject with an ocular disorder or is at risk of having an ocular disorder. In some embodiments, a subject is a subject with at least one symptom associated with a disease or disorder associated with the eye (e.g., an ocular disorder and/or retinal disease). Ocular structures associated with such disorders may include the eye, retina, choroid, lens, cornea, trabecular meshwork, iris, optic nerve, optic nerve head, sclera, anterior or posterior segment, or ciliary body, for example. In some embodiments, a subject has an ocular disorder associated with trabecular meshwork (TM) cells, ciliary epithelium cells, retinal cell, or another cell type of the eye. In some embodiments, a subject has at least one mutation associated with at least one of DHX38, DRAM2, KIZ, RDH11, and/or TTLL5. In some embodiments, a subject has at least one mutation associated with at least one of RPE65, NR2E3, GUCY2D CACNA1F, and/or ABCA4; at least one of USH2A, CEP290, and/or ABCA4; at least one of RPE65, PRPH2, PRPF31, BEST1, and/or ABCA4; at least one of RPGR, RPE65, RHO, NR2E3 GUCA1A, and/or CRX; at least one of USH2A, RPGR, RPE65, RLBP1, RHO, and/or PRPH2; at least one of GUCY2D, CRB1, AIPL1, and/or ABCA4; at least one of RPE65, PRPH2, NR2E3 GUCY2D, GUCA1A, and/or CRX; at least one of USH2A, RPGR, RPE65, RLBP1, RHO, and/or PRPH2; at least one of ABCA4, AIPL1, BEST1, CACNA1F, CEP290, and/or CFH; or at least one mutation associated with at least one of RHO, NR2E3, and/or GUCA1A. In some embodiments, a subject has a disease associated with at least one of the following genes (e.g., mutation): GUCY2D (Guanylate Cyclase 2D, Retinal), PRPH2 (Peripherin 2), GUCA1A (Guanylate Cyclase Activator 1A), CACNA1F (Calcium Voltage-Gated Channel Subunit Alpha1 F), RHO (Rhodopsin), RPE65 (Retinoid Isomerohydrolase RPE65), ABCA4 (ATP Binding Cassette Subfamily A Member 4), CFH (Complement Factor H), RPGR (Retinitis Pigmentosa GTPase Regulator), and/or PDE6B (Phosphodiesterase 6B).

In certain embodiments, the subject treated in accordance with the methods described herein is female. In certain embodiments, the subject treated in accordance with the methods described herein is male. In certain embodiments, the subject treated in accordance with the methods described herein is a child. In certain embodiments, the subject treated in accordance with the methods described herein is a juvenile subject (e.g., 18 years or younger). In certain embodiments, the subject treated in accordance with the methods described herein is 1 month old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or 5 years old. In certain embodiments, the subject treated in accordance with the methods described herein is less than 1.5 months old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or less than 5 years old. In another specific embodiment, the subject treated in accordance with the methods described herein is 1-2 months old, 2-3 months old, 3-4 months old, 4-5 months old, 5-6 months old, 6-7 months old, 7-8 months old, 8-9 months old, 9-10 months old, 10-11 months old, 11 months to 1 year old, 1-1.5 years old, 1.5-2 years old, 2-2.5 years old, 2.5-3 years old, 3-3.5 years old, 3.5-4 years old, 4-4.5 years old, or 4.5-5 years old. In another specific embodiment, the subject treated in accordance with the methods described herein is 6 months to 5 years old. In another embodiment, the subject treated in accordance with the methods described herein is a human adult over 18 years old. In some embodiments, the subject treated in accordance with the methods described herein is a human child under 18 years. In some embodiments, the subject treated in accordance with the methods described herein is a human child under 84 months of age.

In a specific embodiment, the subject is an adult (at least age 16). In another specific embodiment, the subject is an adolescent (age 12-15). In another specific embodiment, the subject is a child (under age 12). In some embodiments, the subject is under age 6, under age 10, under age 15, under age 18, under age 21, under age 25, under age 30, under age 35, under age 40, under age 45, under age 50, under age 55, under age 60, under age 65, under age 70, under age 75, under age 80, under age 85, under age 90, or under age 95. In some embodiments, the subject is over age 6, over age 10, over age 15, over age 18, over age 21, over age 25, over age 30, over age 35, over age 40, over age 45, over age 50, over age 55, over age 60, over age 65, over age 70, over age 75, over age 80, over age 85, over age 90, or over age 95. In some embodiments, the subject is a subject who is not responsive to a previous treatment. In some embodiments, the subject is a subject who has not received a treatment for a disease or disorder associated with the eye. In some embodiments, the subject is a subject who is currently undergoing treatment for a disease or disorder associated with the eye. In some embodiments, the subject is a subject who has undergone surgery. In some embodiments, the subject is a subject who has received chemotherapy.

### 5.4.2 Administration and Dosage

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56th ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the disclosure that is effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials can be done in accordance with common methodologies known to one skilled in the art, and the optimal dosages and routes of administration as well as toxicity profiles of agents of the disclosure can be established. For example, a clinical trial can be designed to test an rAAV molecule of the disclosure for efficacy and toxicity in human patients.

Toxicity and efficacy of the prophylactic and/or therapeutic agents of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, prophylactic and/or therapeutic agents exhibit large therapeutic indices. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

An rAAV molecule of the disclosure generally is administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies within a range of circulating concentrations that include the ED50 with little or no toxicity. The dosage may vary within this range depending upon the dosage form employed and the route of administration utilized.

In some embodiments, a therapeutically effective dosage of an rAAV vector is from about 0.1 ml to about 100 ml of solution containing concentrations of from about 1×10⁹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the disclosure can include a single treatment or can include a series of treatments. For example, pharmaceutical compositions comprising an agent of the disclosure may be administered once a day, twice a day, or three times a day. In some embodiments, the agent may be administered once a day, every other day, once a week, twice a week, once every two weeks, once a month, once every six weeks, once every two months, twice a year, or once per year. In some embodiments, an rAAV of the disclosure increase or decrease over the course of treatment.

In some embodiments, ongoing treatment is indicated, e.g., on a long-term basis, such as in the ongoing treatment and/or management of ocular disease. For example, in particular embodiments, an rAAV of the disclosure is administered over a period of time, e.g., for at least 6 months, at least one year, at least two years, at least five years, at least ten years, at least fifteen years, at least twenty years, or for the rest of the lifetime of a subject in need thereof.

The rAAV molecules of the disclosure may be administered alone or in combination with other prophylactic and/or therapeutic agents. Each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the different prophylactic and/or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart, or no more than 48 hours apart. In certain embodiments, two or more agents are administered within the same patient visit.

In another specific embodiment, agents of the disclosure may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the disclosure are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the disclosure can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Int'l. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref. Biomed. Eng., 14:20, 1987; Buchwald et al., Surgery, 88:507, 1980; and Saudek et al., N. Engl. J. Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e.g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

The route of administration of an rAAV described herein and the amount of rAAV to be administered to the human patient can be determined based on the severity of the disease, condition of the human patient and the knowledge of the treating physician. In some embodiments, an rAAV, an isolated nucleic acid of the disclosure, or a pharmaceutical composition of the disclosure is delivered directly to the eye of a subject. In some embodiments, an rAAV, an isolated nucleic acid of the disclosure, or a pharmaceutical composition of the disclosure is delivered intravitreally to a subject. In some embodiments, an rAAV, an isolated nucleic acid of the disclosure, or a pharmaceutical composition of the disclosure is a single dose, one time treatment. In some embodiments, an rAAV, an isolated nucleic acid of the disclosure, or a pharmaceutical composition of the disclosure is delivered by ocular tissue injection such as periocular, conjunctival, subtenon, intracameral, intravitreal, intraocular, anterior or posterior juxtascleral, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal. In some embodiments, an rAAV, an isolated nucleic acid of the disclosure, or a pharmaceutical composition of the disclosure is delivered via suprachoroidal administration.

In certain embodiments of the method described herein, a therapeutically effective dose of the recombinant AAV or a pharmaceutical composition of the disclosure is administered to the eye of a subject. In some embodiments, a therapeutically effective dose of the recombinant AAV or a pharmaceutical composition of the disclosure is administered (1) to the subretinal space without vitrectomy (e.g., via the suprachoroidal space or via peripheral injection), (2) to the suprachoroidal space, (3) to the outer space of the sclera (i.e., juxtascleral administration), (4) to the subretinal space via vitrectomy, or (5) to the vitreous cavity, in a volume ranging from 50-100 μl, 100-500 μl, 50 μl to 1000 μl. In some embodiments, the volume is 100-300 μl. In some embodiments, the volume is 250 μl. In some embodiments, the volume depends on the administration method or the route of administration. In certain embodiments, therapeutically effective doses of the recombinant vector or the pharmaceutical composition of the disclosure are administered suprachoroidally in a volume of 100 μl or less, for example, in a volume of 50-100 μl. In certain embodiments, therapeutically effective doses of the recombinant AAV or the pharmaceutical composition of the disclosure are administered to the outer surface of the sclera (e.g., by a posterior juxtascleral depot procedure) in a volume of 500 μl or less, for example, in a volume of 10-20 μl, 20-50 μl, 50-100 μl, 100-200 μl, 200-300 μl, 300-400 μl, or 400-500 μl. In certain embodiments, therapeutically effective doses of the recombinant AAV or the pharmaceutical composition of the disclosure are administered to the subretinal space via peripheral injection in a volume of 50-100 μl, 100-500 μl, 100-300 μl, or 200 μl. In some embodiments, therapeutically effective doses of the recombinant AAV or the pharmaceutical composition of the disclosure are administered subretinally in a volume of 200 μL.

In certain embodiment, described herein is an micro volume injector delivery system, which is manufactured by Altaviz (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery.

In some embodiments, the micro volume injector delivery system that can be used for micro volume injector is a micro volume injector with dose guidance and can be used with, for example, a suprachoroidal needle (for example, the Clearside® needle), a subretinal needle, an intravitreal needle, a juxtascleral needle, a subconjunctival needle, and/or intraretinal needle. In some embodiments, some of benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41g needle can be used for subretinal delivery, while the Clearside® needle and the Visionisti OY adaptor can be used for subretinal delivery).

In certain embodiments of the methods described herein, the recombinant AAV or a pharmaceutical composition of the disclosure is administered suprachoroidally (e.g., by suprachoroidal injection). In some embodiments, suprachoroidal administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13:20-23; Goldstein, 2014, Retina Today 9 (5): 82-87; Baldassarre et al., 2017; each of which is incorporated by reference herein in its entirety). The suprachoroidal drug delivery devices that can be used to deposit the recombinant AAV or a pharmaceutical composition of the disclosure in the suprachoroidal space according to the disclosure described herein include, but are not limited to, suprachoroidal drug delivery devices manufactured by Clearside® Biomedical, Inc. (see, for example, Hariprasad, 2016, Retinal Physician 13:20-23) and MedOne suprachoroidal catheters. In another embodiment, the suprachoroidal drug delivery device that can be used in accordance with the methods described herein comprises the micro volume injector delivery system, which is manufactured by Altaviz (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Currently available technologies for suprachoroidal space (SCS) delivery exist. Preclinically, SC injections have been achieved with scleral flap technique, catheters and standard hypodermic needles, as well as with microneedles. A hollow-bore 750 μm-long microneedle (Clearside Biomedical, Inc.) can be inserted at the pars, and has shown promise in clinical trials. A microneedle designed with force-sensing technology can be utilized for SC injections, as described by Chitnis, et al. (Chitnis, G. D., et al. A resistance-sensing mechanical injector for the precise delivery of liquids to target tissue. Nat Biomed Eng 3, 621-631 (2019). https://doi.org/10.1038/s41551-019-0350-2). Oxular Limited is developing a delivery system (Oxulumis) that advances an illuminated cannula in the suprachoroidal space. The Orbit device (Gyroscope) is a specially-designed system enabling cannulation of the suprachoroidal space with a flexible cannula. A microneedle inside the cannula is advanced into the subretinal space to enable targeted dose delivery. Ab interno access to the SCS can also be achieved using micro-stents, which serve as minimally-invasive glaucoma surgery (MIGS) devices. Examples include the CyPass® Micro-Stent (Alcon, Fort Worth, Texas, US) and iStent® (Glaukos), which are surgically implanted to provide a conduit from the anterior chamber to the SCS to drain the aqueous humor without forming a filtering bleb. Other devices contemplated for suprachoroidal delivery include those described in UK Patent Publication No. GB 2531910A and U.S. Pat. No. 10,912,883 B2.

In another embodiment, the suprachoroidal drug delivery device that can be used in accordance with the methods described herein is a tool that comprises a normal length hypodermic needle with an adaptor (and preferably also a needle guide) manufactured by Visionisti OY, which adaptor turns the normal length hypodermic needle into a suprachoroidal needle by controlling the length of the needle tip exposing from the adapter (see, for example, U.S. Design Pat. No. D878,575; and International Patent Application. Publication No. WO/2016/083669) In a specific embodiment, the suprachoroidal drug delivery device is a syringe with a 1 millimeter 30 gauge needle. During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of therapeutic product from all retinal cell layers and choroidal cells. Using this type of device and procedure allows for a quick and easy in-office procedure with low risk of complications. In some embodiments, a max volume of 100 μl is injected into the suprachoroidal space.

In certain embodiments of the methods described herein, the recombinant AAV or a pharmaceutical composition of the disclosure is administered subretinally (e.g., via vitrectomy). Subretinal administration via vitrectomy is a surgical procedure performed by trained retinal surgeons that involves a vitrectomy with the subject under local anesthesia, and subretinal injection of the gene therapy into the retina (see, e.g., Campochiaro et al., 2017, Hum Gen Ther 28 (1): 99-111, which is incorporated by reference herein in its entirety).

In certain embodiments of the methods described herein, the recombinant vector is administered subretinally without vitrectomy.

In certain embodiments of the methods described herein, the subretinal administration without vitrectomy is performed via the suprachoroidal space by use of a subretinal drug delivery device. In certain embodiments, the subretinal drug delivery device is a catheter which is inserted and tunneled through the suprachoroidal space around to the back of the eye during a surgical procedure to deliver drug to the subretinal space. This procedure allows the vitreous to remain intact and thus, there are fewer complication risks (less risk of gene therapy egress, and complications such as retinal detachments and macular holes), and without a vitrectomy, the resulting bleb may spread more diffusely allowing more of the surface area of the retina to be transduced with a smaller volume. The risk of induced cataract following this procedure is minimized, which is desirable for younger patients. Moreover, this procedure can deliver bleb under the fovea more safely than the standard transvitreal approach, which is desirable for patients with inherited retinal diseases effecting central vision where the target cells for transduction are in the macula. This procedure is also favorable for patients that have neutralizing antibodies (Nabs) to AAVs present in the systemic circulation which may impact other routes of delivery (such as suprachoroidal and intravitreal). Additionally, this method has shown to create blebs with less egress out the retinotomy site than the standard transvitreal approach. The subretinal drug delivery device originally manufactured by Janssen Pharmaceuticals, Inc. now by Orbit Biomedical Inc. (see, for example, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1) can be used for such purpose. In another specific embodiment, the subretinal administration without vitrectomy is performed via peripheral injection into the retina (i.e., peripheral to the optic disc, fovea and macula located in the back of the eye). This can be accomplished by transvitreal injection.

In one embodiment, a sharp needle is inserted into the sclera via the superior or inferior side of the eye (e.g., at the 2 or 10 o'clock position) so that the needle passes all the way through the vitreous to inject the retina on the other side. In another embodiment, a trochar is inserted into the sclera to allow a subretinal cannula to be inserted into the eye. The cannula is inserted through the trochar and through the vitreous to the area of desired injection. In either embodiment, the recombinant AAV is injected in the subretinal space, forming a bleb containing the recombinant vector on the opposite inner surface of the eye. Successful injection is confirmed by the appearance of a dome shaped retinal detachment/retinal bleb.

A self-illuminating lens may be used as a light source for the transvitreal administration (see e.g., Chalam et al., 2004, Ophthalmic Surgery and Lasers 35:76-77, which is incorporated by reference herein in its entirety). Alternatively, one or more trocar(s) can be placed for light (or infusion) if desired. In yet another embodiment, an optic fiber chandelier can be utilized via a trocar for visualizing the subretinal injection. In certain embodiments, delivery to the subretinal or suprachoroidal space can be performed using the methods and/or devices described and disclosed in International Publication Nos. WO 2016/042162, WO 2017/046358, WO 2017/158365, and WO 2017/158366, each of which is incorporated by reference in its entirety.

In some embodiments, one, two, or more peripheral injections can be performed to administer the recombinant AAV or a pharmaceutical composition of the disclosure. In this way, one, two, or more blebs containing recombinant AAV can be made in the subretinal space peripheral to the optic disc, fovea and macula. Surprisingly, while administration of the recombinant vector is confined to the peripherally injected blebs, expression of the therapeutic product throughout the retina can be detected when using this approach.

In some embodiments, the intravitreal administration is performed with a intravitreal drug delivery device that comprises the micro volume injector delivery system, which is manufactured by Altaviz (see, e.g. International Patent Application Publication No. WO 2013/177215), United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. The micro volume injector is a micro volume injector with dose guidance and can be used with, for example, a intravitreal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for subretinal delivery).

In certain embodiments, the peripheral injection results in uniform expression of the therapeutic product throughout the eye (e.g. the expression level at the site of injection varies by less than 5%, 10%, 20%, 30%, 40%, or 50% as compared to the expression level at other areas of the eye). The expression of the therapeutic product throughout the eye can be measured by any method known in the art for such a purpose, for example, by whole mount immunofluorescent staining of the eye or retina, or by immunofluorescent staining on frozen ocular sections.

In the event that a transvitreal injection results in loss of the recombinant AAV in the vitreous instead of the subretinal space, an optional vitrectomy can be performed to remove the recombinant vector that was injected into the vitreous. A subretinal injection with vitrectomy can then be performed to deliver the 250 μl of recombinant AAV into the subretinal space. Alternatively, if some of the injected recombinant AAV is deposited into the vitreous and a vitrectomy is not performed to remove the recombinant vector from the vitreous, a catheter lined with immobilized (e.g., covalently bound) anti-AAV antibodies (e.g., anti AAV9 antibodies), can be inserted into the vitreous to capture and remove excess recombinant vector from the vitreous.

In a specific embodiment, the subretinal administration is performed with a subretinal drug delivery device that comprises the micro volume injector delivery system, which is manufactured by Altaviz (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Micro volume injector is a micro volume injector with dose guidance and can be used with, for example, a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for suprachoroidal delivery).

In certain embodiments, the recombinant AAV or a pharmaceutical composition of the disclosure is administered to the outer surface of the sclera (for example, by the use of a juxtascleral drug delivery device that comprises a cannula, whose tip can be inserted and kept in direct apposition to the scleral surface). In a specific embodiment, administration to the outer surface of the sclera is performed using a posterior juxtascleral depot procedure, which involves drug being drawn into a blunt-tipped curved cannula and then delivered in direct contact with the outer surface of the sclera without puncturing the eyeball. In particular, following the creation of a small incision to bare sclera, the cannula tip is inserted. The curved portion of the cannula shaft is inserted, keeping the cannula tip in direct apposition to the scleral surface. After complete insertion of the cannula, the drug is slowly injected while gentle pressure is maintained along the top and sides of the cannula shaft with sterile cotton swabs. This method of delivery avoids the risk of intraocular infection and retinal detachment, side effects commonly associated with injecting therapeutic agents directly into the eye.

In a specific embodiment, the juxtascleral administration is performed with a juxtascleral drug delivery device that comprises the micro volume injector delivery system, which is manufactured by Altaviz (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Micro Volume Injector is a micro volume injector with dose guidance and can be used with, for example, a juxtascleral needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip.

In certain embodiments, an infrared thermal camera can be used to detect changes in the thermal profile of the ocular surface after the administering of a solution which is cooler than body temperature to detect changes in the thermal profile of the ocular surface that allows for visualization of the spread of the solution, e.g., within the SCS, and can potentially determine whether the administration was successfully completed. This is because in certain embodiments the formulation containing the recombinant vector to be administered is initially frozen, brought to room temperature (68-72° F.), and thawed for a short period of time (e.g., at least 30 minutes) before administration, and thus the formulation is colder than the human eye (about 92° F.) (and sometimes even colder than room temperature) at the time of injection. The drug product is typically used within 4 hours of thaw and the warmest the solution would be is room temperature. In a preferred embodiment, the procedure is videoed with infrared video.

Infrared thermal cameras can detect small changes in temperature. They capture infrared energy through a lens and convert the energy into an electronic signal. The infrared light is focused onto an infrared sensor array which converts the energy into a thermal image. The infrared thermal camera can be used for any method of administration to the eye, including any administration route described herein, for example, suprachoroidal administration, subretinal administration, subconjunctival administration, intravitreal administration, or administration with the use of a slow infusion catheter in to the suprachoroidal space. In a specific embodiment, the infrared thermal camera is an FLIR T530 infrared thermal camera. The FLIR T530 infrared thermal camera can capture slight temperature differences with an accuracy of +3.6° F. The camera has an infrared resolution of 76,800 pixels. The camera also utilizes a 24° lens capturing a smaller field of view. A smaller field of view in combination with a high infrared resolution contributes to more detailed thermal profiles of what the operator is imaging. However, other infrared camera can be used that have different abilities and accuracy for capturing slight temperature changes, with different infrared resolutions, and/or with different degrees of lens.

In a specific embodiment, the infrared thermal camera is an FLIR T420 infrared thermal camera. In a specific embodiment, the infrared thermal camera is an FLIR T440 infrared thermal camera. In a specific embodiment, the infrared thermal camera is an Fluke Ti400 infrared thermal camera. In a specific embodiment, the infrared thermal camera is an FLIRE60 infrared thermal camera. In a specific embodiment, the infrared resolution of the infrared thermal camera is equal to or greater than 75,000 pixels. In a specific embodiment, the thermal sensitivity of the infrared thermal camera is equal to or smaller than 0.05° C. at 30° C. In a specific embodiment, the field of view (FOV) of the infrared thermal camera is equal to or lower than 25°×25°.

In certain embodiments, an iron filer is used with the infrared thermal camera to detect changes in the thermal profile of the ocular surface. In a preferred embodiment, the use of an iron filter is able to a generate pseudo-color image, wherein the warmest or high temperature parts are colored white, intermediate temperatures are reds and yellows, and the coolest or low temperature parts are black. In certain embodiments, other types of filters can also be used to generate pseudo-color images of the thermal profile.

The thermal profile for each administration method can be different. For example, in one embodiment, a successful suprachoroidal injection can be characterized by: (a) a slow, wide radial spread of the dark color, (b) very dark color at the beginning, and (c) a gradual change of injectate to lighter color, i.e., a temperature gradient noted by a lighter color. In one embodiment, an unsuccessful suprachoroidal injection can be characterized by: (a) no spread of the dark color, and (b) a minor change in color localized to the injection site without any distribution. In certain embodiments, the small localized temperature drop is result from cannula (low temperature) touching the ocular tissues (high temperature). In one embodiment, a successful intravitreal injection can be characterized by: (a) no spread of the dark color, (b) an initial change to very dark color localized to the injection site, and (c) a gradual and uniform change of the entire eye to darker color. In one embodiment, an extraocular efflux can be characterized by: (a) quick flowing streams on outside on the exterior surface of the eye, (b) very dark color at the beginning, and (c) a quick change to lighter color.

Because the therapeutic product is continuously produced (under the control of a constitutive promoter or induced by hypoxic conditions when using an hypoxia-inducible promoter), maintenance of lower concentrations can be effective. Vitreous humour concentrations can be measured directly in patient samples of fluid collected from the vitreous humour or the anterior chamber, or estimated and/or monitored by measuring the patient's serum concentrations of the therapeutic product—the ratio of systemic to vitreal exposure to the therapeutic product is about 1:90,000. (E.g., see, vitreous humor and serum concentrations of ranibizumab reported in Xu L, et al., 2013, Invest. Opthal. Vis. Sci. 54:1616-1624, at p. 1621 and Table 5 at p. 1623, which is incorporated by reference herein in its entirety).

Also provided herein are treatment system, devices, and apparatuses to be used for a treatment method described herein, which may comprise one or more of the following: bottles, tubes, light source, and microinjector. In certain embodiments, the light source is a self-illuminating contact lens, which can be used to deposit vector in the back of the eye and in particular and to avoid damaging the optic disc, fovea and/or macula (see, e.g., Chalam et al., 2004, Ophthalmic surgery and lasers. 35. 76-77, which is incorporated by reference herein in its entirety). In certain embodiments, a self-illuminating contact lens is utilized during peripheral injection for visualizing the subretinal injection (see, e.g., Chalam et al., 2004, Ophthalmic surgery and lasers. 35. 76-77, which is incorporated by reference herein in its entirety). In certain embodiments, an optic fiber chandelier is utilized via a second trocar for visualizing the subretinal injection.

In certain embodiments, dosages are measured by genome copies per ml or the number of genome copies administered to the eye of the patient (e.g., administered suprachoroidally, subretinally, intravitreally, juxtasclerally, subconjunctivally, and/or intraretinally). In certain embodiments, 1×10⁹ genome copies per ml to 1×10¹⁵ genome copies per ml are administered. In a specific embodiment, 1×10⁹ genome copies per ml to 1×10¹⁰ genome copies per ml are administered. In another specific embodiment, 1×10¹⁰ genome copies per ml to 1×10¹¹ genome copies per ml are administered. In another specific embodiment, 1×10¹⁰ to 5×10¹¹ genome copies are administered. In another specific embodiment, 1×10¹¹ genome copies per ml to 1×10¹² genome copies per ml are administered. In another specific embodiment, 1×10¹² genome copies per ml to 1×10¹³ genome copies per ml are administered. In another specific embodiment, 1×10¹³ genome copies per ml to 1×10¹⁴ genome copies per ml are administered. In another specific embodiment, 1×10¹⁴ genome copies per ml to 1×10¹⁵ genome copies per ml are administered. In another specific embodiment, about 1×10⁹ genome copies per ml are administered. In another specific embodiment, about 1×10¹⁰ genome copies per ml are administered. In another specific embodiment, about 1×10¹¹ genome copies per ml are administered. In another specific embodiment, about 1×10¹² genome copies per ml are administered. In another specific embodiment, about 1×10¹³ genome copies per ml are administered. In another specific embodiment, about 1×10¹⁴ genome copies per ml are administered. In another specific embodiment, about 1×10¹⁵ genome copies per ml are administered. In certain embodiments, 1×10⁹ to 1×10¹⁵ genome copies are administered. In a specific embodiment, 1×10⁹ to 1×10¹⁰ genome copies are administered. In another specific embodiment, 1×10¹⁰ to 1×10¹¹ genome copies are administered. In another specific embodiment, 1×10¹⁰ to 5×10¹¹ genome copies are administered. In another specific embodiment, 1×10¹¹ to 1×10¹² genome copies are administered. In another specific embodiment, 1×10¹² to 1×10¹³ genome copies are administered. In another specific embodiment, 1×10¹³ to 1×10¹⁴ genome copies are administered. In another specific embodiment, 1×10¹³ to 1×10¹⁴ genome copies are administered. In another specific embodiment, 1×10¹⁴ to 1×10¹⁵ genome copies are administered. In another specific embodiment, about 1×10⁹ genome copies are administered. In another specific embodiment, about 1×10¹⁰ genome copies are administered. In another specific embodiment, about 1×10¹¹ genome copies are administered. In another specific embodiment, about 1×10¹² genome copies are administered. In another specific embodiment, about 1×10¹³ genome copies are administered. In another specific embodiment, about 1×10¹⁴ genome copies are administered. In another specific embodiment, about 1×10¹⁵ genome copies are administered. In certain embodiments, about 3.0×10¹³ genome copies per eye are administered. In certain embodiments, up to 3.0×10¹³ genome copies per eye are administered.

In certain embodiments, about 2.0×10¹⁰ genome copies per eye are administered. In certain embodiments, about 6.0×10¹⁰ genome copies per eye are administered. In certain embodiments, about 1.0×10¹⁰ to 2.0×10¹⁰ genome copies per eye are administered by subretinal injection. In certain embodiments, about 2.0×10¹⁰ to 3.0×10¹⁰ genome copies per eye are administered by subretinal injection. In certain embodiments, about 3.0×10¹⁰ to 4.0×10¹⁰ genome copies per eye are administered by subretinal injection. In certain embodiments, about 4.0×10¹⁰ to 5.0×10¹⁰ genome copies per eye are administered by subretinal injection. In certain embodiments, about 5.0×10¹⁰ to 6.0×10¹⁰ genome copies per eye are administered by subretinal injection. In certain embodiments, about 6.0×10¹⁰ to 7.0×10¹⁰ genome copies per eye are administered by subretinal injection. In certain embodiments, about 7.0×10¹⁰ to 8.0×10¹⁰ genome copies per eye are administered by subretinal injection. In certain embodiments, about 8.0×10¹⁰ to 9.0×10¹⁰ genome copies per eye are administered by subretinal injection. In certain embodiments, about 9.0×10¹⁰ to 1.0×10¹¹ genome copies per eye are administered by subretinal injection. In certain embodiments, about 1.0×10¹¹ to 2.0×10¹¹ genome copies per eye are administered by subretinal injection. In certain specific embodiments, about 2.0×10¹⁰ genome copies per eye are administered by subretinal injection. In certain specific embodiments, about 6.0×10¹⁰ genome copies per eye are administered by subretinal injection. In some embodiments, the injection volume of a subretinal injection is 200 μL.

In certain embodiments, about 2×10¹⁰ to 3×10¹⁰ genome copies per eye are administered by suprachoroidal injection. In certain embodiments, about 3×10¹⁰ to 4×10¹⁰ genome copies per eye are administered by suprachoroidal injection. In certain embodiments, about 4×10¹⁰ to 5×10¹⁰ genome copies per eye are administered by suprachoroidal injection. In certain embodiments, about 5×10¹⁰ to 6×10¹⁰ genome copies per eye are administered by suprachoroidal injection. In certain embodiments, about 6×10¹⁰ to 7×10¹⁰ genome copies per eye are administered by suprachoroidal injection. In certain embodiments, about 7×10¹⁰ to 8×10¹⁰ genome copies per eye are administered by suprachoroidal injection. In certain embodiments, about 8×10¹⁰ to 9×10¹⁰ genome copies per eye are administered by suprachoroidal injection. In certain embodiments, about 9×10¹⁰ to 1×10¹¹ genome copies per eye are administered by suprachoroidal injection. In certain embodiments, about 1×10¹¹ to 2×10¹¹ genome copies per eye are administered by suprachoroidal injection. In certain embodiments, about 2×10¹¹ to 3×10¹¹ genome copies per eye are administered by suprachoroidal injection. In certain embodiments, about 3×10¹¹ to 4×10¹¹ genome copies per eye are administered by suprachoroidal injection. In certain embodiments, about 4×10¹¹ to 5×10¹¹ genome copies per eye are administered by suprachoroidal injection. In certain embodiments, about 5×10¹¹ to 6×10¹¹ genome copies per eye are administered by suprachoroidal injection. In certain embodiments, about 6×10¹¹ to 7×10¹¹ genome copies per eye are administered by suprachoroidal injection. In certain embodiments, about 7×10¹¹ to 8×10¹¹ genome copies per eye are administered by suprachoroidal injection. In certain embodiments, about 8×10¹¹ to 9×10¹¹ genome copies per eye are administered by suprachoroidal injection. In certain embodiments, about 9×10¹¹ to 1×10¹² genome copies per eye are administered by suprachoroidal injection.

In certain embodiments, about 2×10¹⁰ genome copies per eye are administered by suprachoroidal injection. In certain embodiments, about 3×10¹⁰ genome copies per eye are administered by suprachoroidal injection. In certain embodiments, about 4×10¹⁰ genome copies per eye are administered by suprachoroidal injection. In certain embodiments, about 5×10¹⁰ genome copies per eye are administered by suprachoroidal injection. In certain embodiments, about 6×10¹⁰ genome copies per eye are administered by suprachoroidal injection. In certain embodiments, about 7×10¹⁰ genome copies per eye are administered by suprachoroidal injection. In certain embodiments, about 8×10¹⁰ genome copies per eye are administered by suprachoroidal injection. In certain embodiments, about 9×10¹⁰ genome copies per eye are administered by suprachoroidal injection. In certain embodiments, about 1×10¹¹ genome copies per eye are administered by suprachoroidal injection. In certain embodiments, about 1.5×10¹¹ genome copies per eye are administered by suprachoroidal injection. In certain embodiments, about 2×10¹¹ genome copies per eye are administered by suprachoroidal injection. In certain embodiments, about 2.5×10¹¹ genome copies per eye are administered by suprachoroidal injection. In certain embodiments, about 3×10¹¹ genome copies per eye are administered by suprachoroidal injection. In certain embodiments, a single suprachoroidal injection is administered. In certain embodiments, a double suprachoroidal injection is administered. In certain embodiments, the injection volume for a suprachoroidal injection is 100 μl.

### 5.5 Combination Therapies

The methods provided herein may be combined with one or more additional therapies (e.g., therapy to treat or ameliorate a disease or disorder associated with the eye). The additional therapies may be administered before, concurrently or subsequent to the gene therapy treatment of the disclosure.

In some embodiments, an additional therapeutic agent for treating a disease associated with the eye (e.g., retinal disease) can be administered to the subject. For example, at least one of a drug for retinal disease can be administered in combination with an rAAV or a method of the disclosure, such as pioglitazone, hydroxocobalamin, levoleucovorin, methylcobalamin, and/or benserazide (or a comparable drug, or a generic).

In some embodiments, additional therapeutic agents that may be used in a combination therapy with the methods of the disclosure include pegaptanib, ranibizumab, PKC412, nepafenac, bevacizumab, aflibercept, and integrin receptor antagonists (including vitronectin receptor agonists). See, for example, Takahashi et al. (2003) Invest. Ophthalmol. Vis. ScL 44:409-15, Campochiaro et al. (2004) Invest. Ophthalmol. Vis. Sci. 45:922-31, van Wijngaarden et al. (2005) JAMA 293:1509-13, U.S. Pat. No. 6,825,188 to Callahan et al., and U.S. Pat. No. 6,881,736 to Manley et al.; each of which is herein incorporated by reference regarding such compounds.

Examples of additional therapeutic agents that may be used in a combination therapy with the methods of the disclosure include, but are not limited to: A0003, A36 peptide, AAV2-sFLT01, ACE041, ACU02, ACU3223, ACU4429, AdPEDF, aflibercept, AG13958, aganirsen, AGN150998, AGN745, AL39324, AL78898A, AL8309B, ALN-VEG01, alprostadil, AM1101, amyloid beta antibody, anecortave acetate, Anti-VEGFR-2 Alterase, Aptocine, APX003, ARC1905, ARC1905 with Lucentis, ATG3, ATP-binding cassette, sub-family A, member 4 gene, ATXS10, Avastin with Visudyne, AVT101, AVT2, bertilimumab, bevacizumab with verteporfin, bevasiranib sodium, bevasiranib sodium; with ranibizumab, brimonidine tartrate, BVA301, canakinumab, Cand5, Cand5 with Lucentis, CERE140, ciliary neurotrophic factor, CLT009, CNT02476, collagen monoclonal antibody, complement component 5 aptamer (pegylated), complement component 5 aptamer (pegylated) with ranibizumab, complement component C3, complement factor B antibody, complement factor D antibody, copper oxide with lutein, vitamin C, vitamin E, and zinc oxide, dalantercept, DE109, dexamethasone with ranibizumab and verteporfin, disitertide, DNA damage inducible transcript 4 oligonucleotide, E10030, E10030 with Lucentis, EC400, eculizumab, EGP, EHT204, embryonic stem cells, human stem cells, endoglin monoclonal antibody, EphB4 RTK Inhibitor, EphB4 Soluble Receptor, ESBA1008, ETX6991, Evizon, Eyebar, EyePromise Five, Eyevi, Eylea, F200, FCFD4514S, fenretinide, fluocinolone acetonide, fluocinolone acetonide with ranibizumab, fms-related tyrosine kinase 1 oligonucleotide, fms-related tyrosine kinase 1 oligonucleotide with kinase insert domain receptor 169, fosbretabulin tromethamine, Gamunex, GEM220, GS101, GSK933776, HC31496, Human n-CoDeR, HYB676, IBI-20089 with Lucentis, iCo-008, Iconl, I-Gold, Ilaris, Iluvien, Iluvien with Lucentis, immunoglobulins, integrin alpha5betA1 immunoglobulin fragments, Integrin inhibitor, IRIS Lutein, I-Sense Ocushield, Isonep, isopropyl unoprostone, JPE1375, JSM6427, KH902, Lenti Vue, LFG316, LP590, LPO1010AM, Lucentis, Lucentis with Visudyne, Lutein ekstra, Lutein with myrtillus extract, Lutein with zeaxanthin, M200, M200 with Lucentis, Macugen, MC1101, MCT355, mecamylamine, Microplasmin, motexafin lutetium, MP0112, NADPH oxidase inhibitors, Neoretna, neurotrophin 4 gene, Nova21012, Nova21013, NT501, NT503, Nutri-Stulln, ocriplasmin, OcuXan, Oftan Macula, Optrin, ORA102 with Avastin, P144, P17, Palomid 529, PAN90806, Panzem, Panzem, PARP Inhibitors, pazopanib hydrochloride, pegaptanib sodium, PF4523655, PG11047, piribedil, platelet-derived growth factor beta polypeptide aptamer (pegylated), platelet-derived growth factor beta polypeptide aptamer (pegylated) with ranibizumab, PLG101, PMX20005, PMX53, POT4, PRS055, PTK787, ranibizumab, ranibizumab with triamcinolone acetonide, ranibizumabwith verteporfin, ranibizumab with volociximab, RD27, Rescula, Retaane, retinal pigment epithelial cells, RetinoStat, RG7417, RN6G, RT101, RTU007, SB267268, serpin peptidase inhibitor, clade F, member 1 gene, shark cartilage extract, Shefl, SIR1046, SIR1076, Sirna027, sirolimus, SMTD004, Snelvit, SOD Mimetics, Soliris, sonepcizumab, squalamine lactate, ST602, StarGen, T2TrpRS, TA106, talaporfin sodium, Tauroursodeoxycholic acid, TG100801, TM, TLCx99, TRC093, TRC105, triamcinolone acetonide with verteporfin, Trivastal Retard, TT30, Ursa, ursodiol, Vangiolux, VAR10200, vascular endothelial growth factor antibody, vascular endothelial growth factor B, vascular endothelial growth factor kinoid, vascular endothelial growth factor oligonucleotide, VAST Compounds, vatalanib, VEGF Inhibitor, verteporfin, Visudyne, Visudyne with Lucentis and dexamethasone, Visudyne with triamcinolone acetonide, Vivis, volociximab, Votrient, XV615, zeaxanthin, ZFP TF, zinc-monocysteine and Zybrestat.

In some embodiments, the gene therapy of the disclosure is administered in combination with an immune suppression therapy. Immune suppression therapies involving a regimen of tacrolimus or rapamycin (sirolimus) in combination with mycophenolic acid, or other immune suppression regimens procedures can be employed. Such immune suppression treatment may be administered during the course of gene therapy, and in certain embodiments, pre-treatment and/or post treatment with immune suppression therapy can be preferred. Immune suppression therapy can be continued subsequent to the gene therapy treatment, based on the judgment of the treating physician, and may thereafter be withdrawn when immune tolerance is induced; e.g., after 180 days.

In certain embodiments, the methods of treatment provided herein further comprise administering to the human subject an immune suppression regimen comprising prednisolone, mycophenolic acid, and/or tacrolimus. In certain embodiments, the methods of treatment provided herein further comprise administering to the human subject an immune suppression regimen comprising prednisolone, mycophenolic acid, and/or rapamycin (sirolimus). In certain embodiments, the methods of treatment provided herein further comprise administering to the human subject an immune suppression regimen that does not comprise tacrolimus. In certain embodiments, the methods of treatment provided herein further comprise administering to the human subject an immune suppression regimen comprising one or more steroid or corticosteroids such as methylprednisolone and/or prednisolone, as well as tacrolimus and/or sirolimus. In certain embodiments, the immune suppression therapy comprises administering a combination of (a) tacrolimus and/or mycophenolic acid, or (b) rapamycin and/or mycophenolic acid to a subject before or concurrently with the gene therapy and continuing thereafter. In certain embodiments, the immune suppression therapy is withdrawn after 180 days. In certain embodiments, the immune suppression therapy is withdrawn after 30, 60, 90, 120, 150, or 180 days.

### 5.6 Disease Markers and Treatment Assessment

In certain embodiments, efficacy of a treatment method as described herein may be monitored by measuring the levels of at least one biomarker related to a disease or disorder associated with the eye (such as the target genes described in Section 5.4), measuring visual deficits by, for example, BCVA (Best-Corrected Visual Acuity), intraocular pressure, slit lamp biomicroscopy, and/or indirect ophthalmoscopy. In some embodiments, efficacy is determined based on the amelioration of at least one symptom related to a disease or disorder of the eye. In some embodiments, efficacy is measured by detecting homogeneity expression of a transgene in the retina (e.g., by using a fluorescence assay). In some embodiments, efficacy is measured by detecting infectivity of a cell (e.g., by using a fluorescence assay). For example, in some embodiments the variant AAV capsid protein confers increased infectivity of a retinal cell compared to the infectivity of the retinal cell by an AAV virion comprising the corresponding parental AAV capsid protein. In some embodiments, fluorescence assay is fluorescence resonance energy transfer (FRET). In some embodiments, efficacy is measured by determining the infectivity of a retinal cell by an rAAV or variant AAV capsid protein.

In some embodiments, effects of the methods provided herein on visual deficits may be measured by whether the human subject's eye that is treated by a method described herein achieves BCVA of greater than 43 letters post-treatment (e.g., 46-50 weeks or 98-102 weeks post-treatment). A BCVA of 43 letters corresponds to 20/160 approximate Snellen equivalent. In a specific embodiment, the human subject's eye that is treated by a method described herein achieves BCVA of greater than 43 letters post-treatment (e.g., 46-50 weeks or 98-102 weeks post-treatment).

In specific embodiments, effects of the methods provided herein on visual deficits may be measured by whether the human subject's eye that is treated by a method described herein achieves BCVA of greater than 84 letters post-treatment (e.g., 46-50 weeks or 98-102 weeks post-treatment). A BCVA of 84 letters corresponds to 20/20 approximate Snellen equivalent. In a specific embodiment, the human subject's eye that is treated by a method described herein achieves BCVA of greater than 84 letters post-treatment (e.g., 46-50 weeks or 98-102 weeks post-treatment).

Effects of the methods provided herein on physical changes to eye/retina may be measured by SD-OCT (SD-Optical Coherence Tomography). Efficacy may be monitored as measured by electroretinography (ERG). Effects of the methods provided herein may be monitored by measuring signs of vision loss, infection, inflammation and other safety events, including retinal detachment.

Retinal thickness may be monitored to determine efficacy of the methods provided herein. Without being bound by any particular theory, thickness of the retina may be used as a clinical readout. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458).

Effects of the methods provided herein may also be measured by a change from baseline in National Eye Institute Visual Functioning Questionnaire, the Rasch-scored version (NEI-VFQ-28-R) (composite score; activity limitation domain score; and socio-emotional functioning domain score). Effects of the methods provided herein may also be measured by a change from baseline in National Eye Institute Visual Functioning Questionnaire 25-item version (NEI-VFQ-25) (composite score and mental health subscale score). Effects of the methods provided herein may also be measured by a change from baseline in Macular Disease Treatment Satisfaction Questionnaire (MacTSQ) (composite score; safety, efficacy, and discomfort domain score; and information provision and convenience domain score).

In some embodiments, the efficacy of a method described herein is reflected by an improvement in vision at about 4 weeks, 12 weeks, 6 months, 12 months, 24 months, 36 months, or at other desired timepoints. In some embodiments, the improvement in vision is characterized by an increase in BCVA, for example, an increase by 1 letter, 2 letters, 3 letters, 4 letters, 5 letters, 6 letters, 7 letters, 8 letters, 9 letters, 10 letters, 11 letters, or 12 letters, or more. In a specific embodiment, the improvement in vision is characterized by a 5%, 10%, 15%, 20%, 30%, 40%, 50% or more increase in visual acuity from baseline.

In some embodiments, the efficacy of a method described herein is reflected by a reduction in central retinal thickness (CRT) or outer nuclear layer thickness (OLN) at about 4 weeks, 12 weeks, 6 months, 12 months, 24 months, 36 months, or at other desired timepoint, for example, a 5%, 10%, 15%, 20%, 30%, 40%, 50% or more decrease in central retinal thickness from baseline. In some embodiments, there is no inflammation in the eye after treatment or little inflammation in the eye after treatment (for example, an increase in the level of inflammation by 10%, 5%, 2%, 1% or less from baseline).

In some embodiments, effects of the methods provided herein on visual deficits may be measured by OptoKinetic Nystagmus (OKN). Without being bound by theory, this visual acuity screening uses the principles of the OKN involuntary reflex to objectively assess whether a patient's eyes can follow a moving target. By using OKN, no verbal communication is needed between the tester and the patient. As such, OKN can be used to measure visual acuity in pre-verbal and/or non-verbal patients. In certain embodiments, OKN is used to measure visual acuity in patients that are 1 month old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, 5 years old, or older than 5 years old. In certain embodiments, an iPad is used to measure visual acuity through detection of the OKN reflex when a patient is looking at movement on the iPad. If the human patient is a child, visual function can be assessed using an optokinetic nystagmus (OKN)-based approach or a modified OKN-based approach.

In certain embodiments, a visual acuity assessment based on OKN determines that visual acuity improves in a patient after treatment with AAV gene therapy by 5%, 10%, 15%, 20%, 25%, 30%, 35%, 40% 45%, 50%, 55%, 60%, 65%, 70%, 75%, 80%, 85%, 90%, 95% or 100%. In certain embodiments, a visual acuity assessment based on OKN determines that visual acuity does not further deteriorate in a patient after treatment with AAV gene therapy.

Effects of the methods provided herein may also be measured by a change in pupillary light reflex as measured by pupillometry over time, for example, a change in pupillary light reflex a 5%, 10%, 15%, 20%, 30%, 40%, 50%, 60%, 70%, 80%, 90% or more change in pupillary light reflex from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in macular thickness and/or volume as measured by SD-OCT over time, for example, a 5%, 10%, 15%, 20%, 30%, 40%, 50%, 60%, 70%, 80%, 90% or more change in macular thickness and/or volume from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in time to accelerated decline phase of retinal degradation (e.g., reaching 210 μm CRT), for example, a 5%, 10%, 15%, 20%, 30%, 40%, 50%, 60%, 70%, 80%, 90% or more change in time to accelerated decline phase of retinal degradation, or in the time to 30 μm loss in CRT or to CRT of less than about 110 μm, e.g., about 1-2, 2-4, 4-6, 8-10, 10-12, 12-14, 12-16, 16-18, 18-20, 20-22, 22-24 months or about 1, 3, 6, 9, 12, 15, 18, 21, or 24 months to 30 μm loss in CRT or to CRT of less than about 110 μm.

Effects of the methods provided herein may also be measured by a change in SD-OCT anatomical markers, e.g., parafoveal ellipsoid zone, ELM, RNFL, full retinal thickness, the inner nuclear layer, the outer nuclear layer (ONL), the photoreceptor (PR) plus the retinal pigment epithelium (RPE), the outer segment plus the RPE (OS+RPE), and/or the ellipsoid zone (EZ) over time, for example, a 5%, 10%, 15%, 20%, 30%, 40%, 50%, 60%, 70%, 80%, 90% or more change in SD-OCT anatomical markers, e.g., parafoveal ellipsoid zone, ELM, RNFL full retinal thickness, the inner nuclear layer, the outer nuclear layer (ONL), the photoreceptor (PR) plus the retinal pigment epithelium (RPE), the outer segment plus the RPE (OS+RPE), and/or the ellipsoid zone (EZ) from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in fundus appearance on fundus photography over time as measured by the modified WCBS over time, for example, a 5%, 10%, 15%, 20%, 30%, 40%, 50%, 60%, 70%, 80%, 90% or more change in fundus appearance on fundus photography over time as measured by the modified WCBS from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in caregiver-reported visual outcome over time as assessed using PedEyeQ over time, for example, a 5%, 10%, 15%, 20%, 30%, 40%, 50%, 60%, 70%, 80%, 90% or more change in caregiver-reported visual outcome over time as assessed using PedEyeQ over time as measured by the modified WCBS from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in adaptive behaviors based on cognitive ability over time as assessed using the VABs III and MSEL-Visual Reception over time, for example, a 5%, 10%, 15%, 20%, 30%, 40%, 50%, 60%, 70%, 80%, 90% or more change in adaptive behaviors based on cognitive ability over time as assessed using the VABs III and MSEL-Visual Reception over time from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

Effects of the methods provided herein may also be measured by a change in the Hamburg Scale Motor Function score using the treated eye and control eye over time, for example, a 5%, 10%, 15%, 20%, 30%, 40%, 50%, 60%, 70%, 80%, 90% or more change in the Hamburg Scale Motor Function score using the treated eye and control eye over time as measured by the modified WCBS from baseline at about 1-2, 2-4, 4-6, 8-10, 10-12, 12-14, 12-16, 16-18, 18-20, 20-22, 22-24 months or at about 1, 3, 6, 9, 12, 15, 18, 21, or 24 months from baseline.

In some embodiments, vector shedding is determined after administration of an rAAV, pharmaceutical composition, or nucleic acid of the disclosure. In some embodiments, vector shedding may be determined for example by measuring vector DNA in biological fluids such as tears, serum or urine using quantitative polymerase chain reaction. In some embodiments, no vector gene copies are detectable in a biological fluid (e.g., tears, serum or urine) at any time point after administration of an rAAV of the disclosure. In some embodiments, less than 1000, less than 500, less than 100, less than 50 or less than 10 vector gene copies/5 μL are detectable by quantitative polymerase chain reaction in a biological fluid (e.g., tears, serum or urine) at any point after administration. In specific embodiments, 210 vector gene copies/5 μL or less are detectable in serum. In some embodiments, less than 1000, less than 500, less than 100, less than 50 or less than 10 vector gene copies/5 μL are detectable by quantitative polymerase chain reaction in a biological fluid (e.g., tears, serum or urine) by 1, 2, 3, 4, 5, 6, 7, 8, 9, 10, 11, 12, 13 or 14 weeks after administration. In specific embodiments, no vector gene copies are detectable in serum by week 14 after administration of the vector.

In another aspect, expression levels of a transgene (e.g., the transgene encoded by a construct of the disclosure) may be monitored by measuring the transgene product in the serum and/or the CSF of a subject to whom the construct has been administered. Immunogenicity to a construct of the disclosure may be evaluated, assessed as anti-transgene product antibodies (aqueous humor, serum, and CSF), anti-AAV9 antibodies (serum, CSF), and T-cell reactivity to AAV9 and construct transgene product via enzyme-linked immunospot (ELISPOT; whole blood).

### 6. EXAMPLES

Certain embodiments provided herein are illustrated by the following non-limiting examples. The following examples are directed to novel engineered AAV capsids. The invention is illustrated by way of examples, describing the construction of rAAV9 capsids engineered for higher transduction efficiency in the posterior segment of the eye, such as retina and retinal pigment epithelium (RPE). The AAV9-based vectors were constructed with a 10-mer peptide insertion in either VR-IV (after amino acid residue S454) or VR-VIII (after amino acid residue Q588) of AAV9 VP1. In one study, to evaluate the performance of the novel capsids in mouse retina and RPE, AAV of the disclosure (e.g., AAV9 comprising SEQ ID NO: 10 and AAV9 comprising SEQ ID NO: 12) were compared with the parental AAV9 and a control, AAV2-variant capsid.

### 6.1 Example 1. Construction of AAV9 Variants and Assessment of Packaging Efficiency

Several AAV9 mutant capsids were constructed, each including a heterologous peptide but at different insertion points. The heterologous peptides were derived from a nucleolin binding protein (NBP) KDEPQRRSARLSAKPAPPKPEPKPKKAPAKK (SEQ ID NO: 1). The heterogolous peptides were inserted in AAV9 capsids as shown in Table 2. Either full length (with upstream methionine; e.g., SEQ ID NO: 23), or partial NBP peptides: KDEPQRRSARL (SEQ ID NO: 2), SAKPAPPKPE (SEQ ID NO:3), or PKPKKAPAKK (SEQ ID NO: 4), were inserted into an AAV9 capsid gene.

TABLE 2
Peptides inserted in AAV9
capsids used to make rAAV vectors

| Vector # | AAV9 Insertion site/Peptide |
|---|---|
| 2070/ | N-term of VP2/ |
| 1686 | MKDEPQRRSARLSAKPAPPKPEPKPKKAPAKK |
| | (SEQ ID NO: 23) Utilized two plasmids, |
| | 1) peptide + VP2 and 2) VP1/VP3 |
| | expressed separately to form the |
| | AAV9 mutant capsid. |
| 2078 | 533 (after D532)/KDEPQRRSARL |
| | (SEQ ID NO: 2) |
| 2079 | 533 (after D532)/SAKPAPPKPE |
| | (SEQ ID NO: 3) |
| 2080 | 533 (after D532)/PKPKKAPAKK |
| | (SEQ ID NO: 4) |
| 2081 | 589 (after Q588)/KDEPQRRSARL |
| | (SEQ ID NO: 2) |
| 2082 | 589 (after Q588)/SAKPAPPKPE |
| | (SEQ ID NO: 3) |
| 2083 | 589 (after Q588)/PKPKKAPAKK |
| | (SEQ ID NO: 4) |
| 2084 | 455 (after S454)/KDEPQRRSARL |
| | (SEQ ID NO: 2) |
| 2085 | 455 (after S454)/SAKPAPPKPE |
| | (SEQ ID NO: 3) |
| 2086 | 455 (after S454)/PKPKKAPAKK |
| | (SEQ ID NO: 4) |

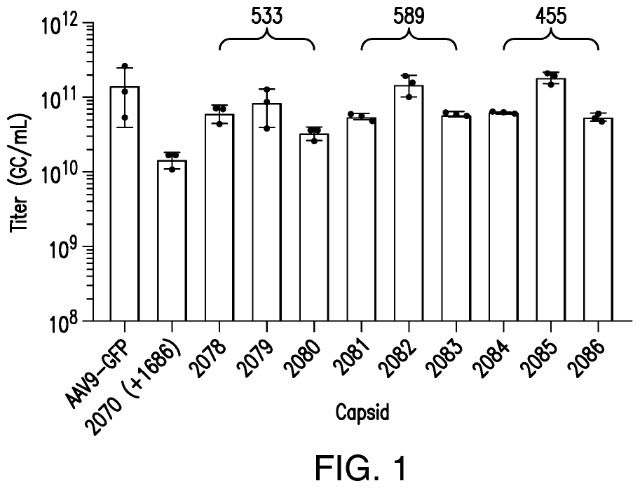

In brief, vectors from Table 2 were packaged with GFP transgene, titered following transfection, and harvested from crude lysate of small flask cell cultures. Titer was determined by polyA qPCR. Assessment of titer from cells infected with viral vectors facilitated determining which insertion points did not interrupt capsid packaging and function. Results showed that several candidates packaged with high efficiency equal to or greater than wild type AAV9 (FIG. 1).

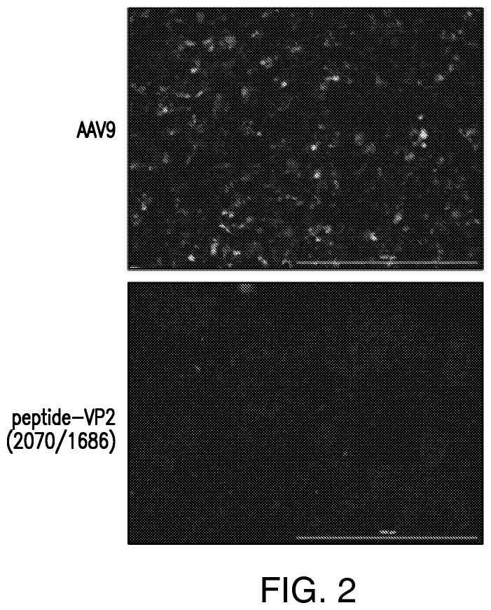

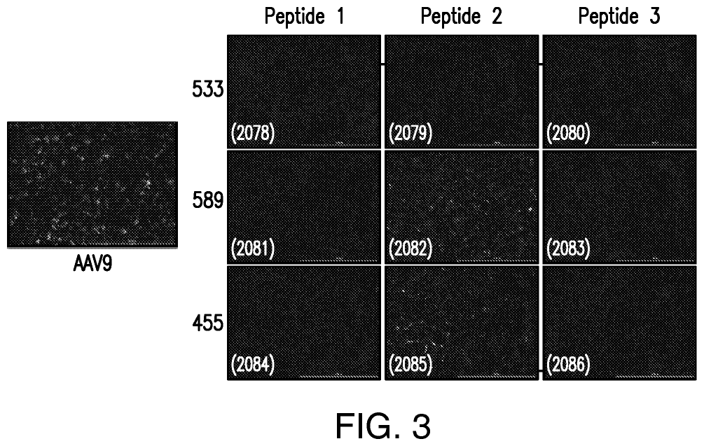

The full nucleolin binding peptide (SEQ ID NO: 1) plus methionine inserted into the N-terminus of VP2 in a pcDNA construct (2070/1686) was expressed separately from VP1/3 of AAV9 and titers observed were low (˜1 log below AAV9 in titer) but detectable (FIG. 1). As can be observed from FIG. 2, GFP expression from vector 2070/1686 is reduced compared to WTAAV9. As such, transduction efficiency is diminished for vector 2070/1686 compared to WTAAV9 (FIG. 2). Expression of GFP in vectors 2078-2086 revealed that vectors 2082 and 2085 behave similarly to WTAAV9 in cultured cells (FIG. 3)

### 6.2 Example 2. Biodistribution of an rAAV Vector Administered Intravitreally in Mice

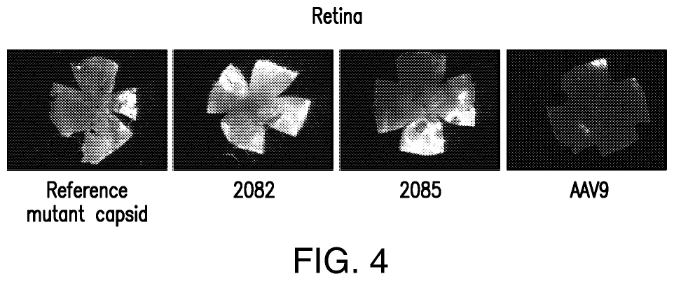

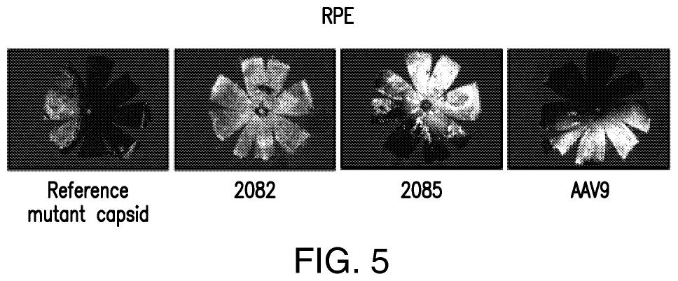

In this experiment, vectors from Table 2 (e.g., vector 2082 [AAV capsid protein corresponding to SEQ ID NO: 11] and 2085 [AAV capsid protein corresponding to SEQ ID NO: 13]) were compared to a reference AAV2-variant and a wild-type AAV9 vector. All recombinant vectors were designed to package a CAG-driven GFP transgene. The vectors were tested in vivo in C57Bl/6 mice (6-8 weeks old, 3 mice per group) following intravitreal (IVT) injection at 1e10 vector genomes (vg) per eye and GFP expression was analyzed by immunostaining with anti-GFP antibody to evaluate the transduction profile of each vector. In brief, retina or RPE flat mounts were treated with primary antibody: Rabbit anti-GFP (Millipore, AB3080) at 1:1000 dilution for 4 days at RT, then treated with secondary antibody donkey anti-Rabbit-AF-488 (Invitrogen, A21206) 1:500, for 2 days at 4° C., and GFP expressing cells were detected by fluorescence. It was observed that higher GFP expressing cells were present throughout the retina in mice injected IVT with 2082 and 2085, compared to the reference AAV2-variant and WTAAV9 (FIG. 4). Further, images from the RPE layer of mice injected IVT with 2082 and 2085 showed broader expression than AAV2-variant and WTAAV9 in RPE (FIG. 5).

### 6.3 Example 3. Evaluation of the Cell Type Specificity of AAV Variants in Brain

To evaluate both the transduction efficiency as well as cell type specificity of transduction, vector preparations of AAV9, AAV9.BC029 (reference CNS peptide insert), and AAV9.2082 (589 (after Q588)/SAKPAPPKPE (SEQ ID NO: 3)) were produced using 2 distinct sets of cis plasmids (e.g., helper plasmid and RepCap plasmid). Thus, packaging 2 distinct sets of vector genomes. In brief, a vector genome expressing a unique fluorescent reporter, either GFP, tdTomato, or iRFP670, under the control of the universal CAG reporter and including a unique 20 bp barcode between the fluorescent reporter coding sequence and the polyadenylation signal, was packaged into AAV9 (GFP), AAV9.BC029 (tdTomato), or AAV9.2082 (iRFP) capsids. Then, a vector genome expressing a codon-optimized, human ApoE transgene, under the control of the universal CAG reporter and containing a unique 20 bp barcode between the ApoE coding sequence and the polyadenylation signal (a different set of barcodes than those used in the fluorescent reporter cassettes) was packaged into AAV9, AAV9.BC029, or AAV9.2082 such that ApoE transcripts produced from one of these capsids could be attributed to a transduction event with that capsid.

Following titering of each vector prep by ddPCR using a primer/probe set specific for the polyA sequence, the titer of each fluorescent reporter prep was used to calculate the volume of each prep required to formulate a test article with equimolar concentrations of AAV9.CAG.GFP, AAV9.BC029.CAG.tdTomato, and AAV9.2082.CAG.iRFP, and vector was formulated accordingly. Similarly, the ddPCR titer of each ApoE reporter prep was used to calculate the volume of each prep required to formulate a test article with equimolar concentrations of AAV9.CAG.hcoApoE.BC1, AAV9.BC029.hcoApoE.BC4, and AAV9.2082.hcoApoE.BC5, and vector was formulated accordingly. Finally, these 2 vector pools were pooled such that 90% of total GC in the final preparation were derived from fluorescent reporter preps, with the 10% of contributing GC being from ApoE reporter preps. As such, final “Tricolor-2” formulated vector pool had a titer of 2e13 GC/mL, with each fluorescent reporter prep having an effective concentration of 6e12 GC/mL and each ApoE reporter prep having an effective concentration of 6.6e11 GC/mL.

The pooled Tricolor-2 test article was administered intraparenchymally to adult, cynomolgus macaques by MRI-guided delivery, following a bilateral dosing scheme into both the hippocampus (60 μL of 2e13 GC/mL test article, 1.2e12 GC total) and the putamen (75 μL of 2e13 GC/mL test article, 1.5e12 GC total). A custom 16 gauge, 10 ft. SmartFlow Neuro Ventricular Cannula was used, and Prohance was added prior to cannula fill in order to allow for repeated T1-weighted MRI monitoring of dosing solution delivery. The cannula was first primed with dosing solution, followed by dosing solution hold in the cannula for at least 5 m to control for any vector adsorption to the device. Dosing solution was then eluted, followed by adjustment of flow rate to 2-5 μL/min, cannula was inserted into target region, flow rate reduced to 1 μL/min until complete dose was delivered. Animals were sacrificed 3 weeks post-test article delivery, and brain was collected in 3 mm thick coronal sections; alternating sections were fixed for histological analysis and direct visualization of fluorescent reporter expression or were sampled using a 4 mm punch for nucleic acid analysis. Additional tissues were also collected for nucleic acid analysis. From each punch, DNA and RNA were extracted using a custom workflow such that paired DNA-RNA data could be obtained from the same tissue sample. The DNA and RNA (following conversion to cDNA) were then subjected to amplicon sequencing using the Illumina MiSeq platform after amplification of barcode-containing regions from nucleic acids derived both from the fluorescent and ApoE reporters. Following sequencing, barcode counts were adjusted for test article input concentrations and normalized to 1, such that the fraction of total fluorescent reporter or total ApoE reporter DNA or RNA that was derived from one of the 3 capsids of interest could be determined.

Results of the Tricolor-2 experiment are represented in Tables 3 through 6. By comparing the measures of DNA and RNA for both the ApoE transgene and also the fluorescent marker transgenes for each vector, as well as noting the location of the tissue sample, it was noted that the transduction pattern of the AAV9.2082 vector was more focal to the area of the injection. AAV9.2082 vector had less diffusion or transport to other tissues, compared to wild-type AAV9.

TABLE 3
RAAFI (relative abundance adjusted for input) measure of DNA and ApoE transgene
in right hemisphere tissue samples (intra-putamen administration of 75 μL of
2e13 GC/mL test article, 1.5e12 GC total/injection site)

| Tissue | | | | | | | |
|---|---|---|---|---|---|---|---|
| Punch | Relative | AAV9 | | AAV9.BC029 | | AAV9.2082 | |
| sample | Location | DNA | RNA | DNA | RNA | DNA | RNA |
| Right | Injection | 0.268175 | 0.284015 | 0.379821 | 0.415264 | 0.352005 | 0.300721 |
| putamen | site | | | | | | |
| (1) | | | | | | | |
| (0 mm fis) | | | | | | | |
| Right | Near | 0.24504 | 0.230959 | 0.310369 | 0.297539 | 0.444593 | 0.471503 |
| extreme | injection | | | | | | |
| capsule | site | | | | | | |
| (2) | | | | | | | |
| (7 mm fis) | | | | | | | |
| Right | Adjacent | 0.415608 | 0.405837 | 0.368599 | 0.474773 | 0.215793 | 0.119391 |
| caudate | striatal | | | | | | |
| (1) | | | | | | | |
| (5 mmfis) | | | | | | | |
| Right | Adjacent | 0.490454 | 0.381774 | 0.193438 | 0.488896 | 0.193438 | 0.488896 |
| putamen | striatal | | | | | | |
| (2) | | | | | | | |
| (6.7 mm fis) | | | | | | | |
| Right | Retrograde | 0.444401 | 0.370854 | 0.177479 | 0.418937 | 0.37812 | 0.210209 |
| substantia | transport | | | | | | |
| nigra (1) | | | | | | | |
| (11.7 mm fis) | | | | | | | |

fis = from injection site

TABLE 4
RAAFI (relative abundance adjusted for input) measure of DNA and Fluorescent
Marker transgene RNA in right hemisphere tissue samples (intra-putamen administration
of 75 μL of 2e13 GC/mL test article, 1.5e12 GC total/injection site)

| Tissue | | | | | | | |
|---|---|---|---|---|---|---|---|
| Punch | Relative | AAV9 | | AAV9.BC029 | | AAV9.2082 | |
| sample | Location | DNA | RNA | DNA | RNA | DNA | RNA |
| Right | Injection | 0.21381 | 0.344554 | 0.318508 | 0.388392 | 0.467682 | 0.267054 |
| putamen | site | | | | | | |
| (1) | | | | | | | |
| (0 mm fis) | | | | | | | |
| Right | Near | 0.168334 | 0.25531 | 0.237312 | 0.296894 | 0.594355 | 0.447796 |
| extreme | injection | | | | | | |
| capsule | site | | | | | | |
| (2) | | | | | | | |
| (7 mm fis) | | | | | | | |
| Right | Adjacent | 0.439515 | 0.498712 | 0.357316 | 0.464591 | 0.203169 | 0.036697 |
| caudate | striatal | | | | | | |
| (1) | | | | | | | |
| (5 mm fis) | | | | | | | |
| Right | Adjacent | 0.539065 | 0.501769 | 0.18171 | 0.454382 | 0.279225 | 0.04385 |
| putamen | striatal | | | | | | |
| (2) | | | | | | | |
| (6.7 mm fis) | | | | | | | |
| Right | Retrograde | 0.534399 | 0.547692 | 0.101901 | 0.316488 | 0.3637 | 0.13582 |
| substantia | transport | | | | | | |
| nigra (1) | | | | | | | |
| (11.7 mm fis) | | | | | | | |

fis = from injection site

TABLE 5
RAAFI (relative abundance adjusted for input) measure of DNA and ApoE transgene
RNA in left hemisphere tissue samples (intra-hippocampal administration
of 60 μL of 2e13 GC/mL test article, 1.2e12 GC total/injection site)

| Tissue | | | | | | | |
|---|---|---|---|---|---|---|---|
| Punch | Relative | AAV9 | | AAV9.BC029 | | AAV9.2082 | |
| sample | Location | DNA | RNA | DNA | RNA | DNA | RNA |
| Left | Injection | 0.274945 | 0.298129 | 0.369877 | 0.423483 | 0.355179 | 0.278389 |
| hippocampus | site | | | | | | |
| (1) | | | | | | | |
| (0 mm fis) | | | | | | | |
| Left | Adjacent | 0.436027 | 0.354543 | 0.167482 | 0.399176 | 0.396491 | 0.246281 |
| hippocampus | hippocampal | | | | | | |
| (2) | tissue | | | | | | |
| (6.7 mm fis) | | | | | | | |
| Left | Adjacent | 0.432301 | 0.413918 | 0.273848 | 0.385968 | 0.293851 | 0.200114 |
| amygdala | tissue | | | | | | |
| (9.2 mm fis) | | | | | | | |
| Left | Distal tissue | 0.344217 | 0.407341 | 0.528227 | 0.556824 | 0.127556 | 0.035835 |
| putamen (2) | (same plane | | | | | | |
| (12 mm fis) | as injection | | | | | | |
| | site) | | | | | | |

fis = from injection site

TABLE 6
RAAFI (relative abundance adjusted for input) measure of DNA and fluorescent marker
transgene RNA in left hemisphere tissue samples (intra-hippocampal administration
of 60 μL of 2e13 GC/mL test article, 1.2e12 GC total/injection site)

| Tissue | | | | | | | |
|---|---|---|---|---|---|---|---|
| Punch | Relative | AAV9 | | AAV9.BC029 | | AAV9.2082 | |
| sample | Location | DNA | RNA | DNA | RNA | DNA | RNA |
| Left | Injection | 0.217473 | 0.302913 | 0.321954 | 0.480247 | 0.460573 | 0.21684 |
| hippocampus | site | | | | | | |
| (1) | | | | | | | |
| 0 mm fis | | | | | | | |
| Left | Adjacent | 0.481484 | 0.334224 | 0.121746 | 0.553984 | 0.39677 | 0.111792 |
| hippocampus | hippocampal | | | | | | |
| (2) | tissue | | | | | | |
| (6.7 mm fis) | | | | | | | |
| Left | Adjacent | 0.483863 | 0.377005 | 0.22335 | 0.511374 | 0.292787 | 0.111621 |
| amygdala | tissue | | | | | | |
| (9.2 mm fis) | | | | | | | |
| Left | Distal tissue | 0.405442 | 0.518501 | 0.485106 | 0.459613 | 0.109452 | 0.021886 |
| putamen (2) | (same plane | | | | | | |
| (12 mm fis) | as injection | | | | | | |
| | site) | | | | | | |

fis = from injection site

### 6.4 Example 4. Evaluation of Transgene Expression Following Delivery of AAV Variants by IVT

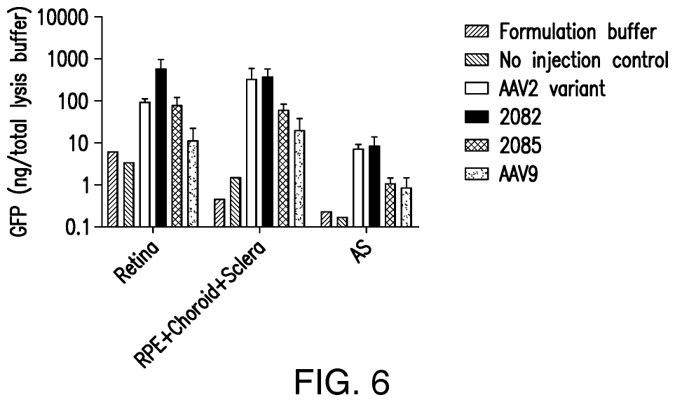

To evaluate transgene expression in various ocular tissues via delivery of engineered capsids carrying eGFP transgene, AAV9, an AAV2 variant (SEQ ID NO: 24), AAV9.2082 (SAKPAPPKPE introduced after position Q588) and AAV9.2085 (SAKPAPPKPE introduced after position S454) vectors were dosed at 2e9 GC/eye and administered IVT to adult male C5BL6/J mice. At 3 weeks post-injection, tissues were collected and eGFP level was measured by ELISA. Whole eyeballs, retina, RPE+choroid+sclera, and anterior segment (AS) samples were collected at three weeks post-injection (3 WPI). The total eGFP levels were measured by ELISA on extracts from retina, RPE+choroid+sclera, and AS samples. AAV9.2082 transduced the retina at a higher level than AAV9.2085 and AAV2.variant, and AAV9.2082 transduced RPE/Choroid/Sclera and anterior segment (AS) at a comparable level to the AAV2 variant (FIG. 6).

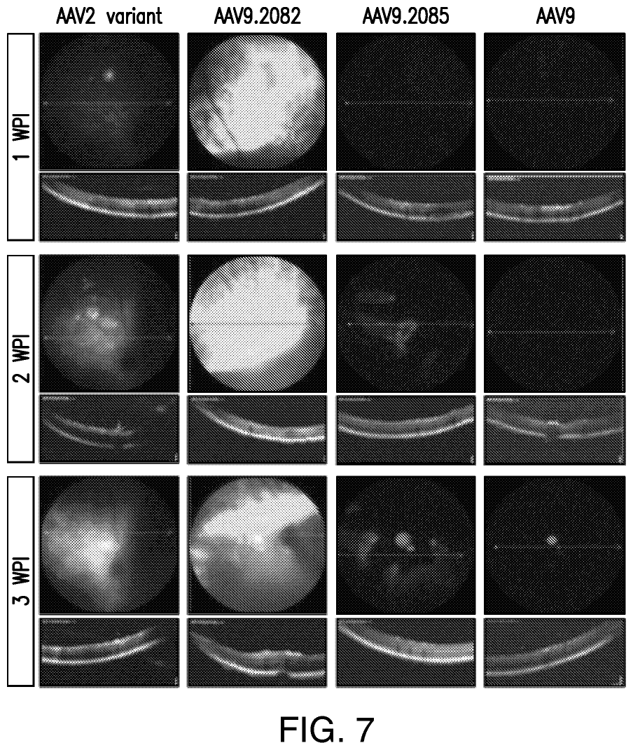

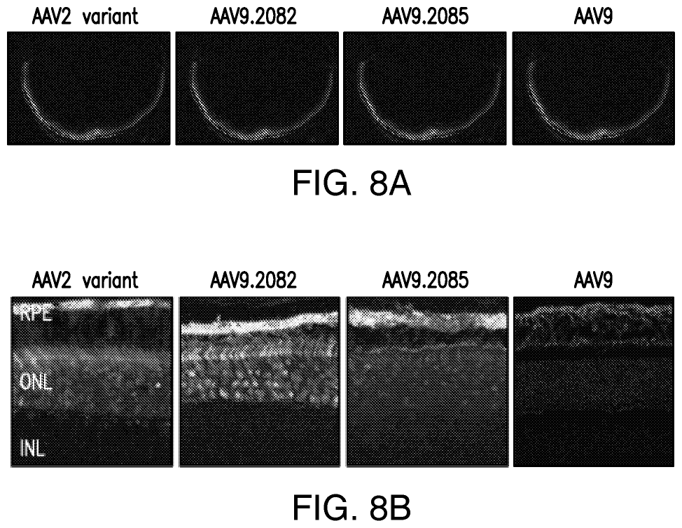

The time course of eGFP transgene expression in the retina was evaluated by weekly fundus auto-fluorescence imaging using a Phoenix MICRON IV retinal imaging microscope. Optical coherence tomography (OCT) images indicate fine integrity of ocular structures 1 to 3 weeks post IVT injection at 2e9 GC/eye. Stronger eGFP expression was visible in AAV9.2082-dosed animals at 2e9 GC/eye, by 1 week post-injection (1 WPI) as evaluated by fundus autofluorescence compared to AAV2-variant capsid (FIG. 7). OCT imaging also indicates an intact ocular structural integrity at 3 WPI with AAV9.2082 and AAV9.2085 at 2e9 GC/eye. The transgene expression was also detected and characterized by immunohistochemistry on whole eye frozen sections and confocal tile-scans of retinal cross-sections immunolabelled for GFP, RPE65, Rhodopsin and DAPI (detected by different color staining, FIGS. 8A-8B). FIGS. 8A-8B show immunolabelled cross-sections of mouse retina infected with AAV2 variant, AAV9.2082, AAV9.2085, and AAV9, with a focus on the RPE, ONL, and INL, stained for GFP, RPE65, Rhodopsin, and DAPI at 3 weeks post IVT injection at 2×10⁹ GC/eye. The retinal cross-section immunolabelling indicated that AAV9.2082 transduced mainly RPE and outer nuclear layer (ONL), whereas AAV9.2085 transduced mostly the RPE cell layer.

TABLE OF SEQUENCES

| SEQ | | |
|---|---|---|
| ID NO. | DESCRIPTION | SEQUENCE |
| 1 | Heterologous sequence | KDEPQRRSARLSAKPAPPKPEPKPKKAPAKK |
| 2 | Heterologous sequence | KDEPQRRSARL |
| 3 | Heterologous sequence | SAKPAPPKPE |
| 4 | Heterologous sequence | PKPKKAPAKK |
| 5 | AAV9 wild-type (WT) | MAADGYLPDWLEDNLSEGIREWWALKPGAPQPKANQQHQD |
| | capsid protein | NARGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQAQAQTGWVQNQG |
| | | ILPGMVWQDRDVYLQGPIWAKIPHTDGNFHPSPLMGGFGM |
| | | KHPPPQILIKNTPVPADPPTAFNKDKLNSFITQYSTGQVS |
| | | VEIEWELQKENSKRWNPEIQYTSNYYKSNNVEFAVNTEGV |
| | | YSEPRPIGTRYLTRNL |
| 6 | AAV9 capsid | atggctgccg atggttatct tccagattgg |
| | | ctcgaggaca accttagtga aggaattcgc |
| | | gagtggtggg ctttgaaacc tggagcccct |
| | | caacccaagg caaatcaaca acatcaagac |
| | | aacgctcgag gtcttgtgct tccgggttac |
| | | aaataccttg gacccggcaa cggactcgac |
| | | aagggggagc cggtcaacgc agcagacgcg |
| | | gcggccctcg agcacgacaa ggcctacgac |
| | | cagcagctca aggccggaga caacccgtac |
| | | ctcaagtaca accacgccga cgccgagttc |
| | | caggagcggc tcaaagaaga tacgtctttt |
| | | gggggcaacc tcgggcgagc agtcttccag |
| | | gccaaaaaga ggcttcttga acctcttggt |
| | | ctggttgagg aagcggctaa gacggctcct |
| | | ggaaagaaga ggcctgtaga gcagtctcct |
| | | caggaaccgg actcctccgc gggtattggc |
| | | aaatcgggtg cacagcccgc taaaaagaga |
| | | ctcaatttcg gtcagactgg cgacacagag |
| | | tcagtcccag accctcaacc aatcggagaa |
| | | cctcccgcag ccccctcagg tgtgggatct |
| | | cttacaatgg cttcaggtgg tggcgcacca |
| | | gtggcagaca ataacgaagg tgccgatgga |
| | | gtgggtagtt cctcgggaaa ttggcattgc |
| | | gattcccaat ggctggggga cagagtcatc |
| | | accaccagca cccgaacctg ggccctgccc |
| | | acctacaaca atcacctcta caagcaaatc |
| | | tccaacagca catctggagg atcttcaaat |
| | | gacaacgcct acttcggcta cagcaccccc |
| | | tgggggtatt ttgacttcaa cagattccac |
| | | tgccacttct caccacgtga ctggcagcga |
| | | ctcatcaaca acaactgggg attccggcct |
| | | aagcgactca acttcaagct cttcaacatt |
| | | caggtcaaag aggttacgga caacaatgga |
| | | gtcaagacca tcgccaataa ccttaccagc |
| | | acggtccagg tcttcacgga ctcagactat |
| | | cagctcccgt acgtgctcgg gtcggctcac |
| | | gagggctgcc tcccgccgtt cccagcggac |
| | | gttttcatga ttcctcagta cgggtatctg |
| | | acgcttaatg atggaagcca ggccgtgggt |
| | | cgttcgtcct tttactgcct ggaatatttc |
| | | ccgtcgcaaa tgctaagaac gggtaacaac |
| | | ttccagttca gctacgagtt tgagaacgta |
| | | cctttccata gcagctacgc tcacagccaa |
| | | agcctggacc gactaatgaa tccactcatc |
| | | gaccaatact tgtactatct ctcaaagact |
| | | attaacggtt ctggacagaa tcaacaaacg |
| | | ctaaaattca gtgtggccgg acccagcaac |
| | | atggctgtcc agggaagaaa ctacatacct |
| | | ggacccagct accgacaaca acgtgtctca |
| | | accactgtga ctcaaaacaa caacagcgaa |
| | | tttgcttggc ctggagcttc ttcttgggct |
| | | ctcaatggac gtaatagctt gatgaatcct |
| | | ggacctgcta tggccagcca caaagaagga |
| | | gaggaccgtt tctttccttt gtctggatct |
| | | ttaatttttg gcaaacaagg aactggaaga |
| | | gacaacgtgg atgcggacaa agtcatgata |
| | | accaacgaag aagaaattaa aactactaac |
| | | ccggtagcaa cggagtccta tggacaagtg |
| | | gccacaaacc accagagtgc ccaagcacag |
| | | gcgcagaccg gctgggttca aaaccaagga |
| | | atacttccgg gtatggtttg gcaggacaga |
| | | gatgtgtacc tgcaaggacc catttgggcc |
| | | aaaattcctc acacggacgg caactttcac |
| | | ccttctccgc tgatgggagg gtttggaatg |
| | | aagcacccgc ctcctcagat cctcatcaaa |
| | | aacacacctg tacctgcgga tcctccaacg |
| | | gccttcaaca aggacaagct gaactctttc |
| | | atcacccagt attctactgg ccaagtcagc |
| | | gtggagatcg agtgggagct gcagaaggaa |
| | | aacagcaagc gctggaaccc ggagatccag |
| | | tacacttcca actattacaa gtctaataat |
| | | gttgaatttg ctgttaatac tgaaggtgta |
| | | tatagtgaac cccgccccat tggcaccaga |
| | | tacctgactc gtaatctgta a |
| 7 | AAV8 WT capsid | MAADGYLPDWLEDNLSEGIREWWALKPGAPKPKANQQKQD |
| | protein | DGRGLVLPGYKYLGPFNGLDKGEPVNAADAAALEHDKAYD |
| | | QQLQAGDNPYLRYNHADAEFQERLQEDTSFGGNLGRAVFQ |
| | | AKKRVLEPLGLVEEGAKTAPGKKRPVEPSPQRSPDSSTGI |
| | | GKKGQQPARKRLNFGQTGDSESVPDPQPLGEPPAAPSGVG |
| | | PNTMAAGGGAPMADNNEGADGVGSSSGNWHCDSTWLGDRV |
| | | ITTSTRTWALPTYNNHLYKQISNGTSGGATNDNTYFGYST |
| | | PWGYFDFNRFHCHFSPRDWQRLINNNWGFRPKRLSFKLFN |
| | | IQVKEVTQNEGTKTIANNLTSTIQVFTDSEYQLPYVLGSA |
| | | HQGCLPPFPADVFMIPQYGYLTLNNGSQAVGRSSFYCLEY |
| | | FPSQMLRTGNNFQFTYTFEDVPFHSSYAHSQSLDRLMNPL |
| | | IDQYLYYLSRTQTTGGTANTQTLGFSQGGPNTMANQAKNW |
| | | LPGPCYRQQRVSTTTGQNNNSNFAWTAGTKYHLNGRNSLA |
| | | NPGIAMATHKDDEERFFPSNGILIFGKQNAARDNADYSDV |
| | | MLTSEEEIKTTNPVATEEYGIVADNLQQQNTAPQIGTVNS |
| | | QGALPGMVWQNRDVYLQGPIWAKIPHTDGNFHPSPLMGGF |
| | | GLKHPPPQILIKNTPVPADPPTTFNQSKLNSFITQYSTGQ |
| | | VSVEIEWELQKENSKRWNPEIQYTSNYYKSTSVDFAVNTE |
| | | GVYSEPRPIGTRYLTRNL |
| 8 | SAKPAPPKPE | TCCGCAAAGCCAGCGCCACCAAAGCCCGAG |
| | nucleotide sequence | |
| 9 | Modified AAV9 capsid | ATGGCTGCCGATGGTTATCTTCCAGATTGGCTCGAGGACA |
| | (VP1) nucleotide | ACCTTAGTGAAGGAATTCGCGAGTGGTGGGCTTTGAAACC |
| | sequence | TGGAGCCCCTCAACCCAAGGCAAATCAACAACATCAAGAC |
| | (SAKPAPPKPE | AACGCTCGAGGTCTTGTGCTTCCGGGTTACAAATACCTTG |
| | peptide inserted at aa | GACCCGGCAACGGACTCGACAAGGGGGAGCCGGTCAACGC |
| | position 589/VR-VIII; | AGCAGACGCGGCGGCCCTCGAGCACGACAAGGCCTACGAC |
| | shown as bold and | CAGCAGCTCAAGGCCGGAGACAACCCGTACCTCAAGTACA |
| | underlined) | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTGCACAGCCCGCTAAAAAGAGACTCAATTTCG |
| | | GTCAGACTGGCGACACAGAGTCAGTCCCAGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGAAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGTGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAATCCGCAAAGCCAGCGCCACCAAAGCCCGAGGCACAG |
| | | GCGCAGACCGGCTGGGTTCAAAACCAAGGAATACTTCCGG |
| | | GTATGGTTTGGCAGGACAGAGATGTGTACCTGCAAGGACC |
| | | CATTTGGGCCAAAATTCCTCACACGGACGGCAACTTTCAC |
| | | CCTTCTCCGCTGATGGGAGGGTTTGGAATGAAGCACCCGC |
| | | CTCCTCAGATCCTCATCAAAAACACACCTGTACCTGCGGA |
| | | TCCTCCAACGGCCTTCAACAAGGACAAGCTGAACTCTTTC |
| | | ATCACCCAGTATTCTACTGGCC5AAGTCAGCGTGGAGATC |
| | | GAGTGGGAGCTGCAGAAGGAAAACAGCAAGCGCTGGAACC |
| | | CGGAGATCCAGTACACTTCCAACTATTACAAGTCTAATAA |
| | | TGTTGAATTTGCTGTTAATACTGAAGGTGTATATAGTGAA |
| | | CCCCGCCCCATTGGCACCAGATACCTGACTCGTAATCTGT |
| | | AA |
| 10 | Modified AAV9 capsid | MAADGYLPDWLEDNLSEGIREWWALKPGAPQPKANQQHQD |
| | (VP1) protein with | NARGLVLPGYKYLGPGNGLDKGEPVNAADAAALEHDKAYD |
| | SAKPAPPKPE peptide | QQLKAGDNPYLKYNHADAEFQERLKEDTSFGGNLGRAVFQ |
| | insert at aa 589 shown | AKKRLLEPLGLVEEAAKTAPGKKRPVEQSPQEPDSSAGIG |
| | as bold and underlined | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQSAKPAPPKPEAQ |
| | | AQTGWVQNQGILPGMVWQDRDVYLQGPIWAKIPHTDGNFH |
| | | PSPLMGGFGMKHPPPQILIKNTPVPADPPTAFNKDKLNSF |
| | | ITQYSTGQVSVEIEWELQKENSKRWNPEIQYTSNYYKSNN |
| | | VEFAVNTEGVYSEPRPIGTRYLTRNL |
| 11 | Modified AAV9 capsid | ATGGCTGCCGATGGTTATCTTCCAGATTGGCTCGAGGACA |
| | (VP1) nucleotide | ACCTTAGTGAAGGAATTCGCGAGTGGTGGGCTTTGAAACC |
| | sequence (SAKPAPPKPE | TGGAGCCCCTCAACCCAAGGCAAATCAACAACATCAAGAC |
| | peptide inserted at aa | AACGCTCGAGGTCTTGTGCTTCCGGGTTACAAATACCTTG |
| | position 455/VR-IV) | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTGCACAGCCCGCTAAAAAGAGACTCAATTTCG |
| | | GTCAGACTGGCGACACAGAGTCAGTCCCAGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGAAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTTCCGCAAAGCCAGCGCCACCAAAGCCCGAGGGACAGAA |
| | | TCAACAAACGCTAAAATTCAGTGTGGCCGGACCCAGCAAC |
| | | ATGGCTGTCCAGGGAAGAAACTACATACCTGGACCCAGCT |
| | | ACCGACAACAACGTGTCTCAACCACTGTGACTCAAAACAA |
| | | CAACAGCGAATTTGCTTGGCCTGGAGCTTCTTCTTGGGCT |
| | | CTCAATGGACGTAATAGCTTGATGAATCCTGGACCTGCTA |
| | | TGGCCAGCCACAAAGAAGGAGAGGACCGTTTCTTTCCTTT |
| | | GTCTGGATCTTTAATTTTTGGCAAACAAGGAACTGGAAGA |
| | | GACAACGTGGATGCGGACAAAGTCATGATAACCAACGAAG |
| | | AAGAAATTAAAACTACTAACCCGGTAGCAACGGAGTCCTA |
| | | TGGACAAGTGGCCACAAACCACCAGAGTGCCCAAGCACAG |
| | | GCGCAGACCGGCTGGGTTCAAAACCAAGGAATACTTCCGG |
| | | GTATGGTTTGGCAGGACAGAGATGTGTACCTGCAAGGACC |
| | | CATTTGGGCCAAAATTCCTCACACGGACGGCAACTTTCAC |
| | | CCTTCTCCGCTGATGGGAGGGTTTGGAATGAAGCACCCGC |
| | | CTCCTCAGATCCTCATCAAAAACACACCTGTACCTGCGGA |
| | | TCCTCCAACGGCCTTCAACAAGGACAAGCTGAACTCTTTC |
| | | ATCACCCAGTATTCTACTGGCCAAGTCAGCGTGGAGATCG |
| | | AGTGGGAGCTGCAGAAGGAAAACAGCAAGCGCTGGAACCC |
| | | GGAGATCCAGTACACTTCCAACTATTACAAGTCTAATAAT |
| | | GTTGAATTTGCTGTTAATACTGAAGGTGTATATAGTGAAC |
| | | CCCGCCCCATTGGCACCAGATACCTGACTCGTAATCTGTA |
| | | A |
| 12 | Modified AAV9 (VP1) | MAADGYLPDWLEDNLSEGIREWWALKPGAPQPKANQQHQD |
| | protein with | NARGLVLPGYKYLGPGNGLDKGEPVNAADAAALEHDKAYD |
| | SAKPAPPKPE peptide | QQLKAGDNPYLKYNHADAEFQERLKEDTSFGGNLGRAVFQ |
| | insert at aa 455 | AKKRLLEPLGLVEEAAKTAPGKKRPVEQSPQEPDSSAGIG |
| | | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSSAKPAPPKPEGQNQQTLKFSVAGPSN |
| | | MAVQGRNYIPGPSYRQQRVSTTVTQNNNSEFAWPGASSWA |
| | | LNGRNSLMNPGPAMASHKEGEDRFFPLSGSLIFGKQGTGR |
| | | DNVDADKVMITNEEEIKTTNPVATESYGQVATNHQSAQAQ |
| | | AQTGWVQNQGILPGMVWQDRDVYLQGPIWAKIPHTDGNFH |
| | | PSPLMGGFGMKHPPPQILIKNTPVPADPPTAFNKDKLNSF |
| | | ITQYSTGQVSVEIEWELQKENSKRWNPEIQYTSNYYKSNN |
| | | VEFAVNTEGVYSEPRPIGTRYLTRNL |
| 13 | Modified AAV9 (VP1) | MAADGYLPDWLEDNLSEGIREWWALKPGAPQPKANQQHQD |
| | protein with | NARGLVLPGYKYLGPGNGLDKGEPVNAADAAALEHDKAYD |
| | SAKPAPPKPE peptide | QQLKAGDNPYLKYNHADAEFQERLKEDTSFGGNLGRAVFQ |
| | insert at aa 533 shown | AKKRLLEPLGLVEEAAKTAPGKKRPVEQSPQEPDSSAGIG |
| | in bold and underlined | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDSAKPAPPKPERFFPLSGSLIFGKQGTGR |
| | | DNVDADKVMITNEEEIKTTNPVATESYGQVATNHQSAQAQ |
| | | AQTGWVQNQGILPGMVWQDRDVYLQGPIWAKIPHTDGNFH |
| | | PSPLMGGFGMKHPPPQILIKNTPVPADPPTAFNKDKLNSF |
| | | ITQYSTGQVSVEIEWELQKENSKRWNPEIQYTSNYYKSNN |
| | | VEFAVNTEGVYSEPRPIGTRYLTRNL |
| 14 | Modified AAV9 (VP1) | MAADGYLPDWLEDNLSEGIREWWALKPGAPQPKANQQHQD |
| | protein with | NARGLVLPGYKYLGPGNGLDKGEPVNAADAAALEHDKAYD |
| | KDEPQRRSARL peptide | QQLKAGDNPYLKYNHADAEFQERLKEDTSFGGNLGRAVFQ |
| | insert at aa 455 | AKKRLLEPLGLVEEAAKTAPGKKRPVEQSPQEPDSSAGIG |
| | | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSKDEPQRRSARLGQNQQTLKFSVAGPS |
| | | NMAVQGRNYIPGPSYRQQRVSTTVTQNNNSEFAWPGASSW |
| | | ALNGRNSLMNPGPAMASHKEGEDRFFPLSGSLIFGKQGTG |
| | | RDNVDADKVMITNEEEIKTTNPVATESYGQVATNHQSAQA |
| | | QAQTGWVQNQGILPGMVWQDRDVYLQGPIWAKIPHTDGNF |
| | | HPSPLMGGFGMKHPPPQILIKNTPVPADPPTAFNKDKLNS |
| | | FITQYSTGQVSVEIEWELQKENSKRWNPEIQYTSNYYKSN |
| | | NVEFAVNTEGVYSEPRPIGTRYLTRNL |
| 15 | Modified AAV9 (VP1) | MAADGYLPDWLEDNLSEGIREWWALKPGAPQPKANQQHQD |
| | protein with | NARGLVLPGYKYLGPGNGLDKGEPVNAADAAALEHDKAYD |
| | KDEPQRRSARL peptide | QQLKAGDNPYLKYNHADAEFQERLKEDTSFGGNLGRAVFQ |
| | insert at aa 533 shown | AKKRLLEPLGLVEEAAKTAPGKKRPVEQSPQEPDSSAGIG |
| | in bold and underlined | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDKDEPQRRSARLRFFPLSGSLIFGKQGTG |
| | | RDNVDADKVMITNEEEIKTTNPVATESYGQVATNHQSAQA |
| | | QAQTGWVQNQGILPGMVWQDRDVYLQGPIWAKIPHTDGNF |
| | | HPSPLMGGFGMKHPPPQILIKNTPVPADPPTAFNKDKLNS |
| | | FITQYSTGQVSVEIEWELQKENSKRWNPEIQYTSNYYKSN |
| | | NVEFAVNTEGVYSEPRPIGTRYLTRNL |
| 16 | Modified AAV9 capsid | MAADGYLPDWLEDNLSEGIREWWALKPGAPQPKANQQHQD |
| | (VP1) protein with | NARGLVLPGYKYLGPGNGLDKGEPVNAADAAALEHDKAYD |
| | KDEPQRRSARL peptide | QQLKAGDNPYLKYNHADAEFQERLKEDTSFGGNLGRAVFQ |
| | insert at aa 589 shown | AKKRLLEPLGLVEEAAKTAPGKKRPVEQSPQEPDSSAGIG |
| | as bold and underlined | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQKDEPQRRSARLA |
| | | QAQTGWVQNQGILPGMVWQDRDVYLQGPIWAKIPHTDGNF |
| | | HPSPLMGGFGMKHPPPQILIKNTPVPADPPTAFNKDKLNS |
| | | FITQYSTGQVSVEIEWELQKENSKRWNPEIQYTSNYYKSN |
| | | NVEFAVNTEGVYSEPRPIGTRYLTRNL |
| 17 | Modified AAV9 (VP1) | MAADGYLPDWLEDNLSEGIREWWALKPGAPQPKANQQHQD |
| | protein with | NARGLVLPGYKYLGPGNGLDKGEPVNAADAAALEHDKAYD |
| | PKPKKAPAKK peptide | QQLKAGDNPYLKYNHADAEFQERLKEDTSFGGNLGRAVFQ |
| | insert at aa 455 | AKKRLLEPLGLVEEAAKTAPGKKRPVEQSPQEPDSSAGIG |
| | | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSPKPKKAPAKKGQNQQTLKFSVAGPSN |
| | | MAVQGRNYIPGPSYRQQRVSTTVTQNNNSEFAWPGASSWA |
| | | LNGRNSLMNPGPAMASHKEGEDRFFPLSGSLIFGKQGTGR |
| | | DNVDADKVMITNEEEIKTTNPVATESYGQVATNHQSAQAQ |
| | | AQTGWVQNQGILPGMVWQDRDVYLQGPIWAKIPHTDGNFH |
| | | PSPLMGGFGMKHPPPQILIKNTPVPADPPTAFNKDKLNSF |
| | | ITQYSTGQVSVEIEWELQKENSKRWNPEIQYTSNYYKSNN |
| | | VEFAVNTEGVYSEPRPIGTRYLTRNL |
| 18 | Modified AAV9 (VP1) | MAADGYLPDWLEDNLSEGIREWWALKPGAPQPKANQQHQD |
| | protein with | NARGLVLPGYKYLGPGNGLDKGEPVNAADAAALEHDKAYD |
| | PKPKKAPAKK peptide | QQLKAGDNPYLKYNHADAEFQERLKEDTSFGGNLGRAVFQ |
| | insert at aa 533 shown | AKKRLLEPLGLVEEAAKTAPGKKRPVEQSPQEPDSSAGIG |
| | in bold and underlined | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDPKPKKAPAKKRFFPLSGSLIFGKQGTGR |
| | | DNVDADKVMITNEEEIKTTNPVATESYGQVATNHQSAQAQ |
| | | AQTGWVQNQGILPGMVWQDRDVYLQGPIWAKIPHTDGNFH |
| | | PSPLMGGFGMKHPPPQILIKNTPVPADPPTAFNKDKLNSF |
| | | ITQYSTGQVSVEIEWELQKENSKRWNPEIQYTSNYYKSNN |
| | | VEFAVNTEGVYSEPRPIGTRYLTRNL |
| 19 | Modified AAV9 capsid | MAADGYLPDWLEDNLSEGIREWWALKPGAPQPKANQQHQD |
| | (VP1) protein with | NARGLVLPGYKYLGPGNGLDKGEPVNAADAAALEHDKAYD |
| | PKPKKAPAKK peptide | QQLKAGDNPYLKYNHADAEFQERLKEDTSFGGNLGRAVFQ |
| | insert at aa 589 shown | AKKRLLEPLGLVEEAAKTAPGKKRPVEQSPQEPDSSAGIG |
| | as bold and underlined | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQPKPKKAPAKKAQ |
| | | AQTGWVQNQGILPGMVWQDRDVYLQGPIWAKIPHTDGNFH |
| | | PSPLMGGFGMKHPPPQILIKNTPVPADPPTAFNKDKLNSF |
| | | ITQYSTGQVSVEIEWELQKENSKRWNPEIQYTSNYYKSNN |
| | | VEFAVNTEGVYSEPRPIGTRYLTRNL |
| 20 | Modified AAV9 (VP1) | MAADGYLPDWLEDNLSEGIREWWALKPGAPQPKANQQHQD |
| | protein with | NARGLVLPGYKYLGPGNGLDKGEPVNAADAAALEHDKAYD |
| | KDEPQRRSARLSAKPAPP | QQLKAGDNPYLKYNHADAEFQERLKEDTSFGGNLGRAVFQ |
| | KPEPKPKKAPAKK | AKKRLLEPLGLVEEAAKTAPGKKRPVEQSPQEPDSSAGIG |
| | peptide insert at aa | KSGAQPAKKRLNFGQTGDTESVPDPQPIGEPPAAPSGVGS |
| | 455 | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSKDEPQRRSARLSAKPAPPKPEPKPKK |
| | | APAKKGQNQQTLKFSVAGPSNMAVQGRNYIPGPSYRQQRV |
| | | STTVTQNNNSEFAWPGASSWALNGRNSLMNPGPAMASHKE |
| | | GEDRFFPLSGSLIFGKQGTGRDNVDADKVMITNEEEIKTT |
| | | NPVATESYGQVATNHQSAQAQAQTGWVQNQGILPGMVWQD |
| | | RDVYLQGPIWAKIPHTDGNFHPSPLMGGFGMKHPPPQILI |
| | | KNTPVPADPPTAFNKDKLNSFITQYSTGQVSVEIEWELQK |
| | | ENSKRWNPEIQYTSNYYKSNNVEFAVNTEGVYSEPRPIGT |
| | | RYLTRNL |
| 21 | Modified AAV9 (VP1) | MAADGYLPDWLEDNLSEGIREWWALKPGAPQPKANQQHQD |
| | protein with | NARGLVLPGYKYLGPGNGLDKGEPVNAADAAALEHDKAYD |
| | KDEPQRRSARLSAKPAPP | QQLKAGDNPYLKYNHADAEFQERLKEDTSFGGNLGRAVFQ |
| | KPEPKPKKAPAKK | AKKRLLEPLGLVEEAAKTAPGKKRPVEQSPQEPDSSAGIG |
| | peptide insert at aa | KSGAQPAKKRLNFGQTGDTESVPDPQPIGEPPAAPSGVGS |
| | 533 shown in bold and | LTMASGGGAPVADNNEGADGVGSSSGNWHCDSQWLGDRVI |
| | underlined | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDKDEPQRRSARLSAKPAPPKPEPKPKKAP |
| | | AKKRFFPLSGSLIFGKQGTGRDNVDADKVMITNEEEIKTT |
| | | NPVATESYGQVATNHQSAQAQAQTGWVQNQGILPGMVWQD |
| | | RDVYLQGPIWAKIPHTDGNFHPSPLMGGFGMKHPPPQILI |
| | | KNTPVPADPPTAFNKDKLNSFITQYSTGQVSVEIEWELQK |
| | | ENSKRWNPEIQYTSNYYKSNNVEFAVNTEGVYSEPRPIGT |
| | | RYLTRNL |
| 22 | Modified AAV9 capsid | MAADGYLPDWLEDNLSEGIREWWALKPGAPQPKANQQHQD |
| | (VP1) protein with | NARGLVLPGYKYLGPGNGLDKGEPVNAADAAALEHDKAYD |
| | KDEPQRRSARLSAKPAPP | QQLKAGDNPYLKYNHADAEFQERLKEDTSFGGNLGRAVFQ |
| | KPEPKPKKAPAKK | AKKRLLEPLGLVEEAAKTAPGKKRPVEQSPQEPDSSAGIG |
| | peptide insert at aa | KSGAQPAKKRLNFGQTGDTESVPDPQPIGEPPAAPSGVGS |
| | 589 shown as bold and | LTMASGGGAPVADNNEGADGVGSSSGNWHCDSQWLGDRVI |
| | underlined | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQKDEPQRRSARLS |
| | | AKPAPPKPEPKPKKAPAKKAQAQTGWVQNQGILPGMVWQD |
| | | RDVYLQGPIWAKIPHTDGNFHPSPLMGGFGMKHPPPQILI |
| | | KNTPVPADPPTAFNKDKLNSFITQYSTGQVSVEIEWELQK |
| | | ENSKRWNPEIQYTSNYYKSNNVEFAVNTEGVYSEPRPIGT |
| | | RYLTRNL |
| 23 | Heterologous sequence | MKDEPQRRSARLSAKPAPPKPEPKPKKAPAKK |
| 24 | AAV2 variant | MAADGYLPDWLEDTLSEGIRQWWKLKPGPPPPKPAERHKD |
| | (Reference capsid) | DSRGLVLPGYKYLGPFNGLDKGEPVNEADAAALEHDKAYD |
| | | RQLDSGDNPYLKYNHADAEFQERLKEDTSFGGNLGRAVFQ |
| | | AKKRVLEPLGLVEEPVKTAPGKKRPVEHSPVEPDSSSGTG |
| | | KAGQQPARKRLNFGQTGDADSVPDPQPLGQPPAAPSGLGT |
| | | NTMATGSGAPMADNNEGADGVGNSSGNWHCDSTWMGDRVI |
| | | TTSTRTWALPTYNNHLYKQISSQSGASNDNHYFGYSTPWG |
| | | YFDFNRFHCHFSPRDWQRLINNNWGFRPKRLNFKLFNIQV |
| | | KEVTQNDGTTTIANNLTSTVQVFTDSEYQLPYVLGSAHQG |
| | | CLPPFPADVFMVPQYGYLTLNNGSQAVGRSSFYCLEYFPS |
| | | QMLRTGNNFTFSYTFEDVPFHSSYAHSQSLDRLMNPLIDQ |
| | | YLYYLSRTNTPSGTTTQSRLQFSQAGASDIRDQSRNWLPG |
| | | PCYRQQRVSKTSADNNNSEYSWTGATKYHLNGRDSLVNPG |
| | | PAMASHKDDEEKFFPQSGVLIFGKQGSEKTNVDIEKVMIT |
| | | DEEEIRTTNPVATEQYGSVSTNLQRGNLALGETTRPARQA |
| | | ATADVNTQGVLPGMVWQDRDVYLQGPIWAKIPHTDGHFHP |
| | | SPLMGGFGLKHPPPQILIKNTPVPANPSTTFSAAKFASFI |
| | | TQYSTGQVSVEIEWELQKENSKRWNPEIQYTSNYNKSVNV |
| | | DFTVDTNGVYSEPRPIGTRYLTR |

### 7. EQUIVALENTS AND INCORPORATIONS BY REFERENCE

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A recombinant adeno-associated virus (rAAV) vector comprising a variant AAV capsid protein, wherein the variant AAV capsid protein comprises a heterologous amino acid sequence having at least about 90% sequence identity to at least about 10 contiguous amino acids of SEQ ID NO: 23, wherein the heterologous amino acid sequence is inserted at the VR-IV site, the VR-VIII site, or the VR-VI site of an AAV capsid protein.

2. A pharmaceutical composition suitable for administration to the eye of a subject, wherein the pharmaceutical composition comprises a recombinant adeno-associated virus (rAAV) vector comprising a variant AAV capsid protein, wherein the variant AAV capsid protein comprises a heterologous amino acid sequence relative to a corresponding parental AAV capsid protein, wherein the heterologous amino acid sequence comprises at least about 10 contiguous amino acids of SEQ ID NO: 23.

3. A method of treating a disease associated with the eye of a subject, wherein the method comprises administering a recombinant adeno-associated virus (rAAV) vector to the eye of the subject, wherein the rAAV comprises a variant AAV capsid protein, wherein the variant AAV capsid protein comprises a heterologous amino acid sequence relative to a corresponding parental AAV capsid protein, wherein the heterologous amino acid sequence comprises at least about 10 contiguous amino acids of SEQ ID NO: 23.

4. An isolated nucleic acid comprising a nucleotide sequence encoding a variant adeno-associated virus (AAV) capsid protein, wherein the variant AAV capsid protein comprises a heterologous amino acid sequence having at least about 90% sequence identity to at least about 10 contiguous amino acids of SEQ ID NO: 23, wherein the heterologous amino acid sequence is inserted at the VR-IV site, the VR-VIII site, or the VR-VI site of an AAV capsid protein.

5. The rAAV of claim 1, the pharmaceutical composition of claim 2, the method of claim 3, or the isolated nucleic acid of claim 4, wherein the at least about 10 contiguous amino acids of SEQ ID NO: 23 comprises SEQ ID NO: 2.

6. The rAAV of claim 1, the pharmaceutical composition of claim 2, the method of claim 3, or the isolated nucleic acid of claim 4, wherein the at least about 10 contiguous amino acids of SEQ ID NO: 23 consists of SEQ ID NO: 2.

7. The rAAV of claim 1, the pharmaceutical composition of claim 2, the method of claim 3, or the isolated nucleic acid of claim 4, wherein the at least about 10 contiguous amino acids of SEQ ID NO: 23 comprises SEQ ID NO: 3.

8. The rAAV of claim 1, the pharmaceutical composition of claim 2, the method of claim 3, or the isolated nucleic acid of claim 4, wherein the at least about 10 contiguous amino acids of SEQ ID NO: 23 consists of SEQ ID NO: 3.

9. The rAAV of claim 1, the pharmaceutical composition of claim 2, the method of claim 3, or the isolated nucleic acid of claim 4, wherein the at least about 10 contiguous amino acids of SEQ ID NO: 23 comprises SEQ ID NO: 4.

10. The rAAV of claim 1, the pharmaceutical composition of claim 2, the method of claim 3, or the isolated nucleic acid of claim 4, wherein the at least about 10 contiguous amino acids of SEQ ID NO: 23 consists of SEQ ID NO: 4.

11. The rAAV of claim 1, the pharmaceutical composition of claim 2, the method of claim 3, or the isolated nucleic acid of claim 4, wherein the at least about 10 contiguous amino acids of SEQ ID NO: 23 comprises SEQ ID NO: 1.

12. The rAAV of claim 1, the pharmaceutical composition of claim 2, the method of claim 3, or the isolated nucleic acid of claim 4, wherein the at least about 10 contiguous amino acids of SEQ ID NO: 23 consists of SEQ ID NO: 1.

13. The rAAV of claim 1, the pharmaceutical composition of claim 2, the method of claim 3, or the isolated nucleic acid of claim 4, wherein the at least about 10 contiguous amino acids of SEQ ID NO: 23 comprises SEQ ID NO: 23.

14. The rAAV of claim 1, the pharmaceutical composition of claim 2, the method of claim 3, or the isolated nucleic acid of claim 4, wherein the at least about 10 contiguous amino acids of SEQ ID NO: 23 consists of SEQ ID NO: 23.

15. The rAAV of claim 1, the pharmaceutical composition of claim 2, the method of claim 3, or the isolated nucleic acid of claim 4, wherein the heterologous amino acid sequence comprises any one of SEQ ID NOs: 1-4 and 23.

16. The rAAV of claim 1, the pharmaceutical composition of claim 2, the method of claim 3, or the isolated nucleic acid of claim 4, wherein the heterologous amino acid sequence consists of any one of SEQ ID NOs: 1-4 and 23.

17. The rAAV of any one of claims 1, and 5-16, the pharmaceutical composition of any one of claims 2 and 5-12, the method of any one of claims 3 and 5-12, or the isolated nucleic acid of any one of claims 4-16, wherein the at least about 10 contiguous amino acids of SEQ ID NO: 23 is inserted in at least one position selected from positions 455, 533, and/or 589 of AAV9 or the corresponding position in the capsid protein of another AAV serotype.

18. The rAAV of any one of claims 1, and 5-17, the pharmaceutical composition of any one of claims 2 and 5-17, the method of any one of claims 3 and 5-17, or the isolated nucleic acid of any one of claims 4-17, wherein the heterologous amino acid sequence is inserted at the VR-IV site.

19. The rAAV of any one of claims 1, and 5-18, the pharmaceutical composition of any one of claims 2 and 5-18, the method of any one of claims 3 and 5-17, or the isolated nucleic acid of any one of claims 4-18, wherein the heterologous amino acid sequence is inserted at the VR-IV site at amino acid position 455 of a parental AAV9 or a corresponding position in another AAV serotype.

20. The rAAV of any one of claims 1, and 5-17, the pharmaceutical composition of any one of claims 2 and 5-17, the method of any one of claims 3 and 5-17, or the isolated nucleic acid of any one of claims 4-17, wherein the heterologous amino acid sequence is inserted at the VR-VI site.

21. The rAAV of any one of claims 1, 5-17, and 20, the pharmaceutical composition of any one of claims 2, 5-17, and 20, the method of any one of claims 3, 5-17, and 20, or the isolated nucleic acid of any one of claims 4-17 and 20, wherein the heterologous amino acid sequence is inserted at the VR-VI site at amino acid position 533 of a parental AAV9 or a corresponding position in another AAV serotype.

22. The rAAV of any one of claims 1, and 5-17, the pharmaceutical composition of any one of claims 2 and 5-17, the method of any one of claims 3 and 5-17, or the isolated nucleic acid of claim 4-17, wherein the heterologous amino acid sequence is inserted at the VR-VIII site.

23. The rAAV of any one of claims 1, 5-17, and 22, the pharmaceutical composition of any one of claims 2, 5-17, and 22, the method of any one of claims 3, 5-17, and 22, or the isolated nucleic acid of any one of claims 4-17 and 22, wherein the heterologous amino acid sequence is inserted at the VR-VIII site at amino acid position 588 of a parental AAV9 or a corresponding position in another AAV serotype.

24. The rAAV of any one of claims 1 and 5-23, wherein the rAAV is suitable for administration to the eye of a subject.

25. The rAAV of any one of claims 1 and 5-23, the pharmaceutical composition of any one of claims 2 and 5-23, or the method of any one of claims 3, and 5-23, wherein the rAAV further comprises a transgene.

26. The method of claim 25, wherein the method results in a homogeneous expression of the transgene in the retina of the subject after the rAAV vector is administered to the eye of the subject.

27. The method of claim 26, wherein the homogeneous expression is measured using a fluorescence assay.

28. The method of claim 27, wherein the fluorescence assay is fluorescence resonance energy transfer (FRET).

29. The method of any one of claims 3, 5-23, and 25-28, wherein the rAAV is administered intravitreally to the eye of the subject.

30. The rAAV of any one of claims 1, and 5-24, the pharmaceutical composition of any one of claims 2, 5-23, and 25, the method of any one of claims 3, 5-29, and 25-29, or the isolated nucleic acid of any one of claims 4-23, wherein the variant AAV capsid protein confers increased infectivity of a retinal cell compared to the infectivity of the retinal cell by an AAV vector comprising the corresponding parental AAV capsid protein, wherein the AAV vector does not comprise the heterologous amino acid sequence.

31. The rAAV of any one of claims 24, 25, and 30, the pharmaceutical composition of any one of claims 2, 5-23, 25, and 30, or the method of any one of claims 3, 5-30 and 25-30, wherein the subject is a human subject.

32. The rAAV of any one of claims 24, 25, and 30-31, the pharmaceutical composition of any one of claims 2, 5-23, 25, and 30-31, or the method of any one of claims 3, and 5-31 and 25-31, wherein the subject is a subject diagnosed with or suspected of having an ocular disease.

33. The rAAV, the pharmaceutical composition, or the method of claim 32, wherein the ocular disease is a retinal disease.

34. The method of any one of claims 3, and 5-23 and 25-31, wherein the disease is a retinal disease.

35. The rAAV or the pharmaceutical composition of claim 34, or the method of claim 33 or 34, wherein the retinal disease is selected from age-related macular degeneration, retinal vasculitis and retinal infective processes, commotio retinae, diabetic retinopathy, hereditary retinal dystrophies, ischemic insult of retinal neurons and macular edema.

36. The rAAV of any one of claims 1, 5-24, 30-33, and 35, the pharmaceutical composition of any one of claims 2, 5-23, 25, 30-33, and 35, the method of any one of claims 3, and 5-23, and 25-35, or the isolated nucleic acid of any one of claims 4-23 and 30, wherein the variant AAV capsid protein is identical to an AAV9 capsid protein except for the at least about 10 contiguous amino acids of SEQ ID NO: 23.

37. The isolated nucleic acid of any one of claims 4-23, 30, and 36, wherein the isolated nucleic acid further encodes a transgene.

38. The isolated nucleic acid of any one of claims 4-23, 30 and 36-37, wherein the variant capsid protein when present in an AAV vector, provides for increased infectivity of the AAV vector for a retinal cell.

39. The method of any one of claims 3, 5-23, 25, and 30-36, wherein the administering is via subretinal, intravitreal or suprachoroidal administration to the eye of the subject.

40. The pharmaceutical composition of any one of claims 2, 5-23, 30-33, and 35-36, wherein the pharmaceutical composition is suitable for subretinal, intravitreal or suprachoroidal administration to the eye of the subject.

41. The method of claim 25, wherein the method results in higher transgene expression level in a portion of the eye of the subject after the rAAV is administered to the eye of the subject compared to the transgene expression level after an AAV vector comprising the corresponding parental AAV capsid protein and the transgene is administered to the eye of the subject or a comparable subject, wherein the AAV vector comprising the corresponding parental AAV capsid protein and the transgene does not comprise the heterologous amino acid sequence.

42. The method of claim 41, wherein the transgene expression level is higher by about or at least about 5%, 10%, 15%, 20%, 25%, 30%, 35%, 40%, 45%, 50%, 55%, 60%, 65%, 70%, 75%, 80%, 85%, 90%, 95%, 99%, or 100%.

43. The method of claim 41 or 42, wherein the portion of the eye is the retina.

44. The method of claim 41 or 42, wherein the portion of the eye is retinal pigment epithelium (RPE), choroid, and/or sclera.

45. The method of claim 41 or 42, wherein the portion of the eye is the anterior segment.

46. The method of claim 41 or 42, wherein the portion of the eye is a population of photoreceptors.

47. The method of any one of claims 41-46, wherein the rAAV comprises SEQ ID NO: 3 after position Q588 of AAV9.

48. The method of any one of claims 41-46, wherein the rAAV comprises SEQ ID NO: 3 after position S454 of AAV9.

49. The method of any one of claims 41-48, wherein the corresponding parental AAV capsid protein is AAV9 capsid protein.

50. The method of any one of claims 41-49, wherein the rAAV and/or the AAV vector is administered by intravitreal administration.

51. The method of any one of claims 41-50, wherein the ocular structural integrity of the eye remains intact at least about 1 week after the rAAV vector is administered to the eye of the subject as determined by optical coherence tomography (OCT).

52. The method of any one of claims 41-51, wherein the comparable subject has been diagnosed with or is at risk of developing the same disease associated with the eye as the subject.

53. The method of any one of claims 41-52, wherein the transgene expression level is determined by an enzyme linked immunosorbent assay (ELISA).

54. A method of treating a disease associated with the CNS of a subject, wherein the method comprises administering a recombinant adeno-associated virus (rAAV) vector to the CNS of the subject, wherein the rAAV vector comprises a variant AAV capsid protein encapsidating a transgene, wherein the variant AAV capsid protein comprises a heterologous amino acid sequence relative to a corresponding parental AAV capsid protein, wherein the heterologous amino acid sequence comprises at least about 10 contiguous amino acids of SEQ ID NO: 23.

55. The method of claim 54, wherein the rAAV vector is administered intravenously, intrathecally, intracerebroventicularly, or intraparenchymally.

56. The method of claim 55, wherein the parental AAV capsid protein is rAAV9.

57. The method of claim 56, wherein the method results in a focal expression of the transgene in the CNS tissue of the injection site after the rAAV vector is administered via intraparenchymal injection.

58. A recombinant adeno-associated virus (rAAV) vector comprising: a nucleic acid sequence encoding an adeno-associated virus (AAV) capsid protein comprising an amino acid sequence that is at least 95% identical to any one of SEQ ID NOs: 10 and 12-22.

59. The rAAV vector of claim 58, wherein the AAV capsid protein comprises an amino acid sequence that is at least 98% identical to any one of SEQ ID NOs: 10 and 12-22.

60. The rAAV vector of claim 58 or 59, wherein the AAV capsid protein comprises an amino acid sequence that is identical to any one of SEQ ID NOs: 10 and 12-22.

61. The rAAV vector of any one of claims 58-60, wherein the AAV capsid protein comprises an amino acid sequence that is identical to SEQ ID NO: 10.

62. The rAAV vector of any one of claims 58-60, wherein the AAV capsid protein comprises an amino acid sequence that is identical to SEQ ID NO: 12.

63. The rAAV vector of any one of claims 58-60, wherein the nucleic acid sequence encoding the AAV capsid protein comprises a nucleotide sequence that is identical to SEQ ID NO: 9 or 11.

64. The rAAV vector of claim 61, wherein the nucleic acid sequence encoding the AAV capsid protein comprises a nucleotide sequence of SEQ ID NOs: 9.

65. The rAAV vector of claim 62, wherein the nucleic acid sequence encoding the AAV capsid protein comprises a nucleotide sequence of SEQ ID NOs: 11.

66. A pharmaceutical composition suitable for administration to the eye of a subject, wherein the pharmaceutical composition comprises a recombinant adeno-associated virus (rAAV) vector comprising a variant AAV capsid protein, wherein the variant AAV capsid protein comprises an amino acid sequence that is at least 95% identical to any one of SEQ ID NOs: 10 and 12-22.

67. The pharmaceutical composition of claim 66, wherein the variant AAV capsid protein comprises an amino acid sequence that is identical to any one of SEQ ID NOs: 10 and 12-22.

68. The pharmaceutical composition of claim 66 or 67, wherein the variant AAV capsid protein comprises an amino acid sequence that is identical to SEQ ID NO: 10.

69. The pharmaceutical composition of claim 66 or 67, wherein the variant AAV capsid protein comprises an amino acid sequence that is identical to SEQ ID NO: 12.

70. A method of treating a disease associated with the eye of a subject, wherein the method comprises administering a recombinant adeno-associated virus (rAAV) vector to the eye of the subject, wherein the rAAV comprises a variant AAV capsid protein, wherein the variant AAV capsid protein comprises an amino acid sequence that is at least 95% identical to any one of SEQ ID NOs: 10 and 12-22.

71. A method of treating a disease associated with the CNS of a subject, wherein the method comprises administering a recombinant adeno-associated virus (rAAV) vector to the CNS of the subject, wherein the rAAV vector comprises a variant AAV capsid protein encapsidating a transgene, wherein the variant AAV capsid protein comprises an amino acid sequence that is at least 95% identical to any one of SEQ ID NOs: 10 and 12-22.

72. The method of claim 70 or 71, wherein the variant AAV capsid protein comprises an amino acid sequence that is identical to any one of SEQ ID NOs: 10 and 12-22.

73. The method of any one of claims 70-72, wherein the variant AAV capsid protein comprises an amino acid sequence that is identical to SEQ ID NO: 10.

74. The method of any one of claims 70-72, wherein the variant AAV capsid protein comprises an amino acid sequence that is identical to SEQ ID NO: 12.

75. An isolated nucleic acid comprising a nucleotide sequence encoding a variant adeno-associated virus (AAV) capsid protein, wherein the variant AAV capsid protein comprises an amino acid sequence that is at least 95% identical to any one of SEQ ID NOs: 10 and 12-22.

76. The isolated nucleic acid of claim 75, wherein the variant AAV capsid protein comprises an amino acid sequence that is identical to any one of SEQ ID NOs: 10 and 12-22.

77. The isolated nucleic acid of claim 75 or 76, wherein the variant AAV capsid protein comprises an amino acid sequence that is identical to SEQ ID NO: 10.

78. The isolated nucleic acid of claim 75 or 76, wherein the variant AAV capsid protein comprises an amino acid sequence that is identical to SEQ ID NO: 12.

79. An isolated nucleic acid comprising a nucleotide sequence of SEQ ID NO: 9 or 11.

80. An isolated nucleic acid comprising a nucleotide sequence of SEQ ID NO: 9.

81. An isolated nucleic acid comprising a nucleotide sequence of SEQ ID NO: 11.

82. A cell comprising the rAAV vector of any one of claims 58-65.

83. A plasmid comprising a nucleotide sequence of SEQ ID NO: 9 or 11.

84. A plasmid comprising a nucleotide sequence of SEQ ID NO: 9.

85. A plasmid comprising a nucleotide sequence of SEQ ID NO: 11.
